(19) United States
(12) Patent Application Publication
(10) Pub. No.: US 2025/0059575 A1
(43) Pub. Date: Feb. 20, 2025

(54) POLYMERASE ENZYME

(71) Applicant: QIAGEN BEVERLY LLC, Beverly, MA (US)

(72) Inventors: Ryan Charles Heller, Amesbury, MA (US); David M. Schuster, Poolesville, MD (US)

(21) Appl. No.: 17/927,490

(22) PCT Filed: May 25, 2021
(86) PCT No.: PCT/US2021/034027

§ 371 Date: Nov. 23, 2022

Related U.S. Application Data
(60) Provisional application No. 63/030,113, filed on May 26, 2020.

(30) Foreign Application Priority Data
Jul. 8, 2020 (EP) 20184704.3

(51) Int. Cl. C12P 19/34; C12N 9/12

(52) U.S. Cl. CPC C12P 19/34; C12N 9/1252; C12N 9/1276; C12Y 207/07007; C12Y 207/07049

(57) ABSTRACT

The present invention is in the field of molecular biology and is directed to novel reverse transcriptase enzymes and compositions, and to methods and kits for producing, amplifying, or sequencing nucleic acid molecules using these novel reverse transcriptase enzymes or compositions. In particular the Invention relates to a polymerase selected from the group of. a polymerase (O15) as encoded by a nucleic acid according to SEQ ID NO. 9 or a nucleic acid that is at least 98% identical thereto, a polymerase (O15) with the amino acid sequence according to SEQ ID NO: 10 or a polymerase that is at least 90% identical thereto, a polymerase (O57) as encoded by a nucleic acid according to SEQ ID NO. 11 or a nucleic acid that is at least 98% identical thereto, a polymerase (O57) with the amino acid sequence according to SEQ ID NO: 12 or a polymerase that is at least 90% identical thereto. a polymerase (O58) as encoded by a nucleic acid according to SEQ ID NO. 13 or a nucleic acid that is at least 98% identical thereto, and a polymerase (O58) with the amino acid sequence according to SEQ ID NO: 14 or a polymerase that is at least 90% identical thereto.

### TECHNICAL FIELD

The present invention is in the field of molecular biology, in particular in the field of enzymes and more particular in the field of polymerases and in the field of nucleic acid amplification and reverse transcription. The present invention is directed to novel reverse transcriptase enzymes and compositions, and to methods and kits for producing, amplifying, or sequencing nucleic acid molecules, particularly cDNA molecules, using these novel reverse transcriptase enzymes or compositions.

### BACKGROUND ART

The detection, analysis, sequencing, transcription and amplification of nucleic acids are among the most important procedures in modern molecular biology. The application of such procedures for amplification, detection, quantification, sequencing and analysis of RNA is most typically dependent on the conversion of RNA into complementary DNA (cDNA) by reverse transcriptases. The term “reverse transcriptase” describes a class of polymerases characterized as RNA dependent DNA polymerases. Consequently, reverse transcriptases are considered foundational enzymes in molecular biology and are important for many applications, especially including the investigation of gene expression, in the diagnosis and management of infectious agents, such as RNA viruses, and in analysis of disease states including cancers and genetic disorders. Consequently, reverse transcriptases with improved properties, such as higher efficiency, speed, thermal stability, or resistance to inhibitory compounds in sample matrixes that negatively impact reverse transcription will lead to improved analysis of RNA and are highly valued in the areas of diagnostics, human and veterinary health care, agriculture, food safety, environmental monitoring and scientific research.

The primary tools for detecting and quantifying RNA are variants of reverse transcription polymerase chain reaction (RT-PCR), such as quantitative RT-PCR (RT-qPCR) or real-time RT-PCR. Other variants of RT-PCR include digital RT-PCR (dRT-PCR) or digital droplet RT-PCR (ddRT-PCR). In addition, reverse transcriptases are essential for many next-generation RNA sequencing (RNA-Seq) methods for RNA analysis.

The RT-PCR procedure involves two separate molecular syntheses: First, the synthesis of cDNA from an RNA template; and second, the replication of the newly synthesized cDNA through PCR amplification. RT-PCR may be performed under three general protocols: 1) Uncoupled RT-PCR, also referred to as two-step RT-PCR. 2) Single enzyme coupled RT-PCR, also referred to as one-step RT-PCR or continuous RT-PCR, in 35 which a single polymerase is used for both the cDNA generation from RNA as well as subsequent DNA amplification. 3) Two (or more) enzyme coupled RT-PCR, in which a thermolabile retroviral RT synthesizes complementary DNA (cDNA) using an RNA template, and a distinct DNA polymerase, commonly Taq polymerase, for amplification of the DNA product. Commonly, a 5′-3′ nuclease activity, inherent in Taq DNA polymerase, facilitates fluorescent detection by amplification-dependent hydrolysis and dequenching of a fluorescent DNA probe. This is sometimes also referred to as one-step RT-PCR or, alternatively, one-tube RT-PCR.

In uncoupled RT-PCR, reverse transcription is performed as an independent step using buffer and reaction conditions optimal for reverse transcriptase activity. Following cDNA synthesis, an aliquot of the RT reaction product is used as template for PCR amplification with a thermostable DNA polymerase, such as Taq DNA Polymerase, under conditions optimal for PCR amplification.

Coupled RT-PCR provides numerous advantages over uncoupled RT-PCR. Coupled RT-PCR requires less handling of the reaction mixture reagents and nucleic acid products than uncoupled RT-PCR (e.g., opening of the reaction tube for component or enzyme addition in between the two reaction steps), and is therefore less labor-intensive, and time-consuming, and has reduced risk of contamination. Furthermore, coupled RT-PCR also requires less sample, making it especially suitable for applications where the sample amounts are limited (e.g., with FFPE, biopsy, or environmental samples).

Although single-enzyme-coupled RT-PCR is easy to perform, this system is expensive to perform, however, due to the amount of DNA polymerase required. In addition, the single enzyme coupled RT-PCR method has been found to be less sensitive than uncoupled RT-PCR, and limited to polymerizing nucleic acids of less than one kilobase pair in length.

Some inherently thermostable DNA polymerases, e.g. Tth polymerase and Hawk Z05, can be induced to function as reverse transcriptases by modifying the buffer to include manganese rather than the typical magnesium (Myers and Gelfand 1991. Biochemistry 30:7661). Other variants of thermostable DNA polymerases, e.g. those of Thermus (U.S. Pat. No. 5,455,170), Thermatoga and other thermophiles, have been modified by mutagenesis and directed evolution to polymerize DNA from RNA templates (Sauter and Marx 2006. Angew. Chem. Int. Ed. Engl. 45:7633; Kranaster et al. 2010. Biotechnol. J. 5:224; Blatter et al. 2013. Angew. Chem. Int. Ed. Engl. 52:11935). Intron encoded RTs from various thermophilic bacteria have been explored for their potential use in single enzyme RT-PCR (Zhao et al. 2018. RNA 24:183: Mohr et al. 2013. RNA 19:958). Alternatively, mutagenesis of archaeal family B DNA polymerases has resulted in functional proofreading thermostable RTs (Ellefson et al. 2016. Science 352:1590).

Single enzyme magnesium-dependent RT-PCR was enabled by PyroPhage R DNA polymerase. A 588 amino acid sequence was submitted as GenBank Acc. No. AFN99405.1 with the patent filings, i.e. U.S. Pat. No. 8,093,030 and related patents, and presumptively comprises the PyroPhage DNA polymerase. This enzyme has both thermostable reverse transcriptase and DNA polymerase activities. This enzyme, as described in patents (U.S. Pat. No. 8,093,030), proved difficult to manufacture consistently, did not have sufficient RT activity, and was not competitive with the two enzyme systems with regard to ease of use, sensitivity, versatility in target RNAs, time-to-result, functionality in detection using probes or overall reliability.

Overall, none of these alternative thermostable reverse transcriptase/polymerase enzymes has been sufficiently effective in RT-PCR. Consequently, coupled RT-PCR systems with two (or more) enzyme mixes based on Taq polymerase and a thermolabile retroviral RT continue to be the state of the art for the great majority of practitioners and generally show increased sensitivity over the single enzyme system, even when coupled in a single reaction mixture. This effect has been attributed to the higher efficiency of reverse transcriptase in comparison to the reverse transcriptase activity of DNA polymerases (Sellner and Turbett, BioTechniques 25(2):230-234 (1998)).

Although the two-enzyme coupled RT-PCR system is more sensitive than the single-enzyme system, reverse transcriptase has been found to interfere directly with DNA polymerase during the replication of the cDNA, thus reducing the sensitivity and efficiency of this technique (Sellner et al., J. Viol. Methods 40:255-264 (1992)). In order to minimize the number of manual manipulations required for processing large numbers of samples, Sellner et al. attempted to design a system whereby all the reagents required for both reverse transcription and amplification can be added to one tube and a single, non-interrupted thermal cycling program can be performed. Whilst attempting to set up such a one-tube system with Taq polymerase and avian myoblastis virus RT, they noticed a substantial decrease in the sensitivity of detection of viral RNA. They found out a direct interference of reverse transcriptase with Taq polymerase. A variety of solutions to overcome the inhibitory activity of reverse transcriptase on DNA polymerase have been tried, including: increasing the amount of template RNA, increasing the ratio of DNA polymerase to reverse transcriptase, adding modifier reagents that may reduce the inhibitory effect of reverse transcriptase on DNA polymerase (e.g., non homologous tRNA, T4 gene 32 protein, sulphur or acetate-containing molecules), and heat-inactivation of the reverse transcriptase before the addition of DNA polymerase.

All of these modified RT-PCR methods have significant drawbacks, however. Increasing the amount of template RNA is not possible in cases where only limited amounts of sample are available. Individual optimization of the ratio of reverse transcriptase to DNA polymerase is not practicable for ready-to-use reagent kits for one-step RT-PCR. The net effect of currently proposed modifier reagents to releive reverse transcriptase inhibition of DNA polymerization is controversial and in dispute: positive effects due to these reagents are highly dependent on RNA template amounts, RNA composition, or may require specific reverse transcriptase-DNA polymerase combinations (Chandler et al., Appl. and Environm Microbiol. 64(2):669-677 (1998)). Finally, heat inactivation of the reverse transcriptase before the addition of the DNA polymerase negates the advantages of the coupled RT-PCR and carries all the disadvantages of uncoupled RT-PCR systems discussed earlier. Even if a reverse transcriptase is heat inactivated, it still may confer an inhibitory effect on PCR, likely due to binding of heat-inactivated reverse transcriptase to the cDNA template.

Some improvements to reduce the inhibitory effect of reverse transcriptase on the activity of the polymerase have been made, including:
1) In US 2009/0137008 Al, Gong and Wang describe the reduction of the inhibitory effect of reverse transcriptase on DNA polymerase by proteins that bind dsDNA in a non-specific way such as Sso7d, Sac7d, Sac7e or Sso7e and by sulfonic-acid and by sulfonic acid salts.
2) In EP 1050587 B1, Missel et al. describe the reduction of the inhibitory effect of reverse transcriptase on DNA polymerase by homopolymeric nucleic acids.
3) In U.S. Pat. No. 9,758,812 Fang and Missel describe the use of anionic polymers to improve the sensitivity of coupled one-step RT-PCR.

Although the methods described by Gong and Wang, Missel et al., and Fang and Missel respectively, successfully have shown a significant reduction of the inhibitory effect of reverse transcriptase, a further improved specificity and sensitivity of RT-PCR by a more effective reduction of the inhibitory effect of reverse transcriptase is still a need in the art.

The lower temperature reaction conditions required for optimal retroviral RT activity (Yasukawa et al., 2008. J. Biochem. 143:261) is another factor that can limit the efficiency of reverse transcription and efficacy of one-step RT-PCR in detecting certain sequences. This is especially true if the lower temperatures promote formation of unfavorable secondary structures such as hairpins, stem loops, and G quadruplexes that block primer binding and impede nascent strand synthesis on the RNA template (Malboeuf et al. 2001. BioTechniques 30:1074). For highly structured RNA targets, especially common in viral genomes, it would be advantageous to perform cDNA synthesis at higher temperatures so that RNA secondary structures are destabilized and non-specific primer binding is minimized. Additionally, highly thermal stable reverse transcriptases would enable compatibilty with monoclonal antibody (U.S. Pat. No. 5,338,671) or chemical hot-start methods (U.S. Pat. No. 5,773,258) such as those used for PCR amplification polymerases such as Taq DNA polymerase to further improve the specificity and efficiency of one-step RT-PCR. Lastly, highly thermostable reverse transcriptases would enable integration of uracil DNA glycoslyase-medated amplicon carry-over decontamination methods (U.S. Pat. No. 5,683,896) in one-step RT-PCR without the requirement for psychrophilic, heat-labile, uracil DNA glycosylases.

Because of the importance of RT-PCR applications, novel reverse transcriptases with high thermal stability and intrinsic inhibitor resistance that overcome the known draw backs associated with a one-step RT-PCR system, in the form of a generalized ready-to-use composition, which exhibits high specificity and sensitivity, requires a small amount of initial sample, reduces the amount of practitioner manipulation, minimizes the risks of contamination, minimizes the expense of reagents, and maximizes the amount of nucleic acid end product is needed in the art.

### SUMMARY OF THE INVENTION

The present invention solves the aforementioned problem by providing for a polymerase comprising,
a. an N-terminal 5′-3′ nuclease domain,
   i. stemming from Taq polymerase or,
   ii. a polymerase sharing at least 95% amino acid sequence identity with the N-terminal 5′-3′ nuclease domain of Taq polymerase,
b. an adjacent and linked polymerase domain, stemming from a viral family A polymerase, wherein the polymerase domain stems preferably from,
   1. JGI20132J14458_100001622 (1607 amino acids), or a functional fragment that shares at least 98% amino acid sequence identity thereto, and is altered to comprise the following amino acid changes, Q627N, H751Q, Q752K, and V753K, or
   2. Ga0186926_122605 (1595 amino acids), or a functional fragment that shares at least 98% amino acid sequence identity thereto, and is altered to comprise the following amino acid changes, Q627N, H752Q, Q753K, and V754K, or
   3. Ga0080008_15802729 (1619 amino acids) or a functional fragment that shares at least 98% amino acid sequence identity thereto, and is altered to comprise the following amino acid changes, Q628N, H752Q, Q753K, and L754K, or
   4. Ga0079997_11796739 (1608 amino acids), or a functional fragment that shares at least 98% amino acid sequence identity thereto and is altered to comprise the following amino acid changes, Q627N, H752Q, Q753K, and I754K.

The term “functional fragment” refers to the minimum amino acid region and corresponding DNA coding sequence from the herein designated metagenomic viral polyproteins that when expressed in a suitable host in the context of suitable regulatory elements either singularly or with ancillary sequence elements, has detectable RNA-directed DNA polymerase activity.

Herein, the N-terminal 5′-3′ nuclease domain acts also as a processivity enhancing fusion tag for the present inventive construct. It is defined as (i) stemming from Taq polymerase or, a polymerase sharing at least 95% amino acid sequence identity with the N-terminal 5′-3′ nuclease domain of Taq polymerase. As such it is not essential that this polypeptide acts as a nuclease within the inventive construct. Within the present inventive construct the inventors observe that the claimed domain acts similarly to Taq DNA polymerase, where additional interactions between the nuclease domain and the DNA template increases template affinity and improves processivity compared with the N-terminal nuclease deletion (Wang et al., 2004. Nucleic Acids Res. 32:1197; Merkens et al., 1995. Biochim. Biophys. Acta. 1264:243; Murali et al., 1998. Proc. Natl. Acad. Sci. U.S.A. 95:12562).

In an alternative embodiment the N-terminal 5′-3′ nuclease domain is RNase H-like, or from the RNase H superfamily and stems preferably from a N-terminal 5′-3′ nuclease domain,
i. stemming from Taq polymerase or,
ii a polymerase sharing at least 95% amino acid sequence identity with the N-terminal 5′-3′ nuclease domain of Taq polymerase,

In particular the new enzyme shows:
a. increased thermostability;
b. increased thermoreactivity;
c. increased resistance to reverse transcriptase inhibitors;
d. increased ability to reverse transcribe difficult templates;
e. increased speed;
f. increased processivity;
g. increased specificity; or
h. increased sensitivity.

Similar or equivalent sites of corresponding amino acid positions in reverse transcriptases from other species can be mutated to produce thermostable and/or thermoreactive reverse transcriptases as disclosed herein. For example, in some embodiments the present invention provides reverse transcriptases having at least 50% (e.g., 50%, 55%, 60%, 65%, 70%, 75%, 80%, 85%, 90%, 91%, 92%, 93%, 94%, 95%, 96%, etc.) amino acid sequence identity to those SEQ IDs claimed herein.

The present invention is also directed to DNA molecules (preferably vectors) containing a gene or nucleic acid molecule encoding the mutant reverse transcriptases of the present invention and to host cells containing such DNA molecules. Any number of hosts may be used to express the gene or nucleic acid molecule of interest, including prokaryotic and eukaryotic cells. Preferably, prokaryotic cells are used to express the polymerases of the invention. The preferred prokaryotic host according to the present invention is E. coli.

The invention also provides compositions and reaction mixtures for use in reverse transcription of nucleic acid molecules, comprising one or more mutant or modified reverse transcriptase enzymes or polypeptides as disclosed herein. Such compositions may further comprise one or more nucleotides, a suitable buffer, and/or one or more DNA polymerases.

The compositions of the invention may also comprise one or more oligonucleotide primers or terminating agents (e.g., dideoxynucleotides). Such compositions may also comprise a stabilizing agent, such as glycerol or a surfactant. Such compositions may further comprise the use of hot start mechanisms to prevent or reduce unwanted polymerization products during nucleic acid synthesis.

The invention provides in certain embodiments, compositions that include one or more reverse transcriptases of the invention and one or more DNA polymerases for use in amplification reactions. Such compositions may further comprise one or more nucleotides and/or a buffer suitable for amplification. The compositions of the invention may also comprise one or more oligonucleotide primers. Such compositions may also comprise a stabilizing agent, such as glycerol or a surfactant. Such compositions may further comprise the use of one or more hot start mechanisms to prevent or reduce unwanted polymerization products during nucleic acid synthesis.

The invention also relates to certain polymerase domains an their uses:
OS-1622 (576 amino acids) SEQ ID NO. 24 is derived from Locus tag JGI20132J14458_100001622
OP-2605 (577 amino acids) SEQ ID NO. 25 is derived from Locus tag Ga0186926_122605
CS-2729 (577 amino acids) SEQ ID NO. 26 is derived from Locus tag Ga0080008_15802729
PS-6739 (577 amino acids) SEQ ID NO. 27 is derived from Locus tag Ga0079997_11796739

The invention further provides methods for synthesis of nucleic acid molecules using one or more mutant reverse transcriptase enzymes or polypeptides as disclosed herein. In particular, the invention is directed to methods for making one or more nucleic acid molecules, comprising mixing one or more nucleic acid templates (preferably one or more RNA templates and most preferably one or more messenger RNA templates) with one or more reverse transcriptases of the invention and incubating the mixture under conditions sufficient to make a first nucleic acid molecule or molecules complementary to all or a portion of the one or more nucleic acid templates. In some embodiments, the first nucleic acid molecule is a single-stranded cDNA. Nucleic acid templates suitable for reverse transcription according to this aspect of the invention include any nucleic acid molecule or population of nucleic acid molecules (preferably RNA and most preferably mRNA), particularly those derived from a cell or tissue. In some embodiments, cellular sources of nucleic acid templates include, but are not limited to, bacterial cells, fungal cells, plant cells and animal cells.

In certain embodiments, the invention provides methods for making one or more double-stranded nucleic acid molecules. Such methods comprise (a) mixing one or more nucleic acid templates (preferably RNA or mRNA, and more preferably a population of mRNA templates) with one or more reverse transcriptases of the invention; (b) incubating the mixture under conditions sufficient to make a first nucleic acid molecule or molecules complementary to all or a portion of the one or more templates; and (c) incubating the first nucleic acid molecule or molecules under conditions sufficient to make a second nucleic acid molecule or molecules complementary to all or a portion of the first nucleic acid molecule or molecules, thereby forming one or more double-stranded nucleic acid molecules comprising the first and second nucleic acid molecules. Such methods may include the use of one or more DNA polymerases as part of the process of making the one or more double-stranded nucleic acid molecules. The invention also concerns compositions useful for making such double-stranded nucleic acid molecules. Such compositions comprise one or more reverse transcriptases of the invention and optionally one or more DNA polymerases, a suitable buffer, one or more primers, and/or one or more nucleotides.

The invention also provides methods for amplifying a nucleic acid molecule. Such amplification methods comprise mixing the double-stranded nucleic acid molecule or molecules produced as described above with one or more DNA polymerases and incubating the mixture under conditions sufficient to amplify the double-stranded nucleic acid molecule. In a first preferred embodiment, the invention concerns a method for amplifying a nucleic acid molecule, the method comprising (a) mixing one or more nucleic acid templates (preferably one or more RNA or mRNA templates and more preferably a population of mRNA templates) with one or more reverse transcriptases of the invention and with one or more DNA polymerases and (b) incubating the mixture under conditions sufficient to amplify nucleic acid molecules complementary to all or a portion of the one or more templates.

The invention is also directed to methods for reverse transcription of one or more nucleic acid molecules comprising mixing one or more nucleic acid templates, which are preferably RNA or messenger RNA (mRNA) and more preferably a population of mRNA molecules, with one or more reverse transcriptase of the present invention and incubating the mixture under conditions sufficient to make a nucleic acid molecule or molecules complementary to all or a portion of the one or more templates. To make the nucleic acid molecule or molecules complementary to the one or more templates, a primer (e.g., an oligo(dT) primer) and one or more nucleotides are preferably used for nucleic acid synthesis in the 5 to 3 direction. Nucleic acid molecules suitable for reverse transcription according to this aspect of the invention include any nucleic acid molecule, particularly those derived from a prokaryotic or eukaryotic cell. Such cells may include normal cells, diseased cells, transformed cells, established cells, progenitor cells, precursor cells, fetal cells, embryonic cells, bacterial cells, yeast cells, animal cells (including human cells), avian cells, plant cells and the like, or tissue isolated from a plant or an animal (e.g., human, cow, pig, mouse, sheep, horse, monkey, canine, feline, rat, rabbit, bird, fish, insect, etc.). Nucleic acid molecules suitable for reverse transcription may also be isolated and/or obtained from viruses and/or virally infected cells.

The invention further provides methods for amplifying or sequencing a nucleic acid molecule comprising contacting the nucleic acid molecule with a reverse transcriptase of the present invention. In some embodiments, such methods comprise one or more polymerase chain reactions (PCRs). In some embodiments, a reverse transcription reaction is coupled to a PCR, such as in RT-PCR.

The present invention also provides kits for reverse transcription comprising the reverse transcriptase of the present invention in a packaged format. The kit for reverse transcription of the present invention can include, for example, the reverse transcriptase, any conventional constituent necessary for reverse transcription such as a nucleotide primer, at least one dNTP, and a reaction buffer, and optionally a DNA polymerase.

The invention is also directed to kits for use in the methods of the invention. Such kits can be used for making, sequencing or amplifying nucleic acid molecules (single-or double-stranded). The kits of the invention comprise a carrier, such as a box or carton, having in close confinement therein one or more containers, such as vials, tubes, bottles and the like. In certain embodiments of the kits of the invention, a first container contains one or more of the reverse transcriptase enzymes of the present invention. The kits of the invention may also comprise, in the same or different containers, one or more DNA polymerase (preferably thermostable DNA polymerases), one or more suitable buffers for nucleic acid synthesis and one or more nucleotides. Alternatively, the components of the kit may be divided into separate containers (e.g., one container for each enzyme and/or component). The kits of the invention also may comprise instructions or protocols for carrying out the methods of the invention. In preferred kits of the invention, the reverse transcriptases are mutated such that the temperature at which cDNA synthesis occurs is increased. In additional preferred kits of the invention, the enzymes (reverse transcriptases and/or DNA polymerases) in the containers are present at working concentrations.

The present invention also solves the problem by providing for a method for amplifying template nucleic acids comprising contacting the template nucleic acids with a polymerase according to the invention, preferably wherein the method is RT-PCR. That means the polymerases of the invention all have reverse transcriptase activity, as described in U.S. Pat. No. 5,322,770.

The term “reverse transcriptase” describes a class of polymerases characterized as RNA dependent DNA polymerases. All known reverse transcriptase enzymes require a primer to synthesize a DNA transcript from an RNA template. Historically, reverse transcriptase has been used primarily to transcribe mRNA into cDNA which can then be cloned into a vector for further manipulation.

The present invention also solves the problem by providing for a kit comprising a polymerase according to the invention, a vector encoding a polymerase according to the invention, or a transformed host cell comprising the vector according to the invention.

The problem is solved with a viral family A polymerase, or a portion thereof comprising one of the following mutations, selected from the group of.
a. Q627N or Q628N
b. H751Q or H752Q
c. Q752K or Q753K
d. V753K or V754K or L754K or I754K or mutations in similar residues from locally aligned family A polymerases per the amino acid numbering of the Taq nuclease domain-linked polymerases as outlined above.

As used herein, the term “comprising” is to be construed as encompassing both “including” and “consisting of”, both meanings being specifically intended, and hence individually disclosed embodiments in accordance with the present invention.

Herein, and throughout the specification mutations within the amino acid sequence of a polymerase are written in the following form: (i) single letter amino acid as found in wild type polymerase, (ii) position of the change in the amino acid sequence of the polymerase and (iii) single letter amino acid as found in the altered polymerase. So, mutation of a Tyrosine residue in the wild type polymerase to a Valine residue in the altered polymerase at position 409 of the amino acid sequence would be written as Y409V. This is standard procedure in molecular biology.

The invention provides simplified and improved methods for the detection of RNA target molecules in a sample. These methods employ thermostable polymerases to catalyze reverse transcription, second strand cDNA synthesis, and, if desired, amplification by PCR. The methods of the present invention provide RNA reverse transcription and amplification with enhanced specificity and at higher temperatures than previous RNA cloning and diagnostic methods. These methods are adaptable for use in kits for laboratory or clinical analysis.

### BRIEF DESCRIPTIONS OF DRAWINGS

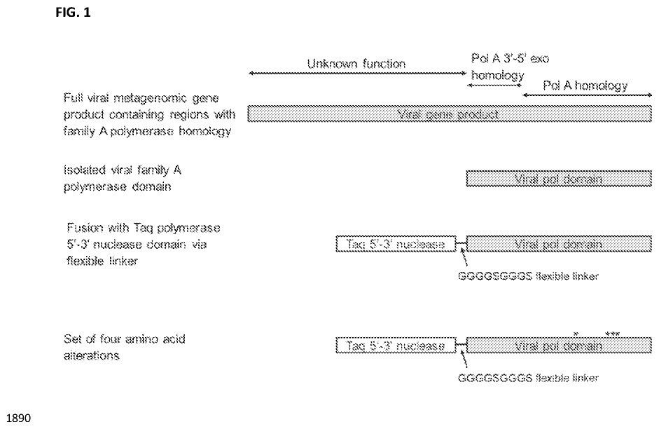

### FIG. 1

Representation of the domain organization of full metagenomic viral gene products containing regions of family A polymerase homology. Core viral polymerase domains were isolated, then fused with the Taq polymerase 5′-3′ nuclease domain at the N-terminus via a flexible linker. Polymerases were further engineered by altering a set of four amino acids for improvements in reverse transcription performance.

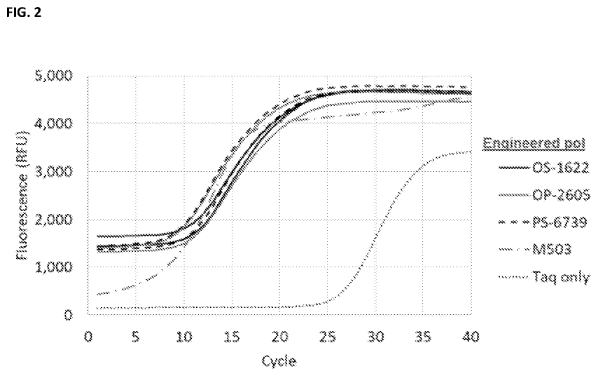

### FIG. 2

FIG. 2 illustrates the efficient reverse transcriptase activity of the engineered viral family A DNA polymerase in lysate-based RT-qPCR reactions using MS2 RNA template and 70° C. reaction temperature compared with the engineered, gene-shuffled M503polymerase.

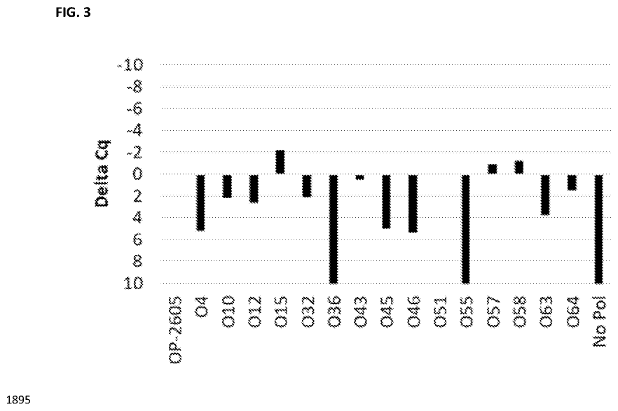

### FIG. 3

FIG. 3 illustrates reverse transcriptase efficiency of OP-2605 mutant library variants after heating at 80° C. for 5 minutes in lysate-based RT-qPCR reactions using MS2 RNA template. The differences in Cq value are reported relative to the parental OP-2605polymerase, in which the absolute Cq value was 20.1. Library variants O15, O57, or O58each generated lower Cq values for detection of MS2 RNA than the parental OP-2605polymerase, indicative of improved sensitivity and corresponding efficiency of RNA conversion to 1st strand product.

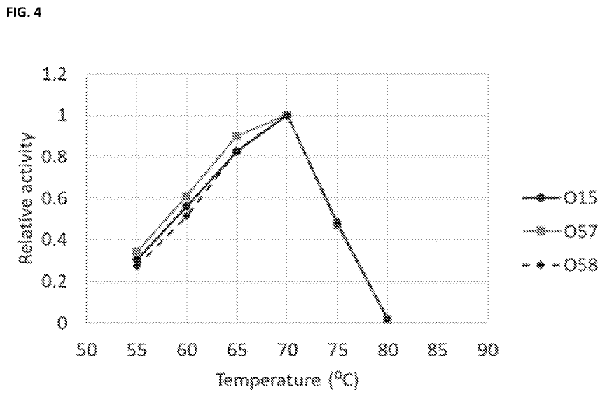

### FIG. 4

FIG. 4 illustrates the thermal activity profile of the engineered viral variants as measured by the relative nucleotide polymerization rates.

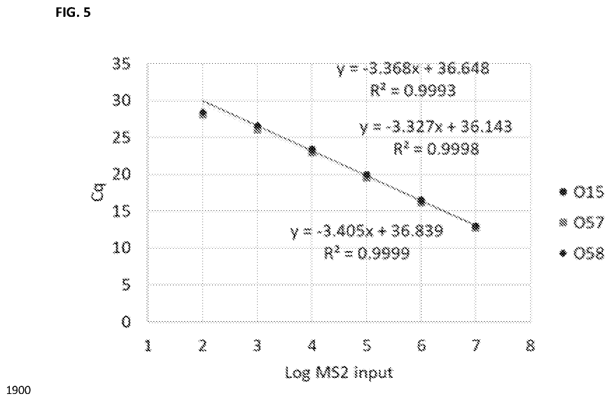

### FIG. 5

FIG. 5 illustrates the sensitivity and efficiency of detection of viral RNA by the engineered viral polymerase variants in probe-based in one-step RT-qPCR reactions.

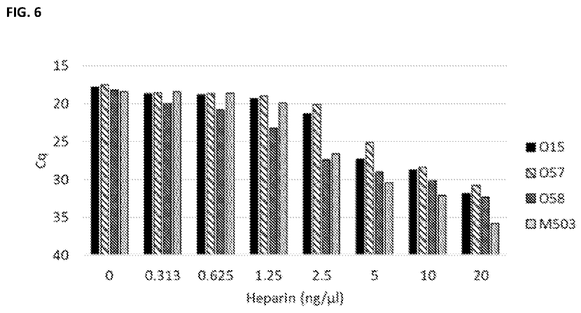

### FIG. 6

FIG. 6 illustrates the heparin resistance of the engineered viral polymerase variants compared with the engineered, gene shuffled M503 polymerase in probe-based, one-step RT-qPCR reactions.

### DETAILED DESCRIPTION OF THE INVENTION

The invention relates to numerous new polymerases, for use in reverse transcription, PCR, sequencing and RT-PCR.

The term “PCR” refers to polymerase chain reaction, which is a standard method in molecular biology for DNA amplification.

“RT-PCR” relates to reverse transcription polymerase chain reaction, a variant of PCR commonly used for the detection and quantification of RNA. RT-PCR comprises two steps, synthesis of complementary DNA (cDNA) from RNA by reverse transcription and amplification of the generated cDNA by PCR. Variants of RT-PCR include quantitative RT-PCR (RT-qPCR), real-time RT-PCR, digital RT-PCR (dRT-PCR) or digital droplet RT-PCR (ddRT-PCR).

“Methods of amplifying RNA without high temperature thermal cycling” as referred to herein, may be isothermal nucleic acid amplification technologies, such as loop-mediated amplification (LAMP), helicase dependent amplification (HDA) and recombinase polymerase amplification (RPA).

As used herein the term “cDNA” refers to a complementary DNA molecule synthesized using a ribonucleic acid strand (RNA) as a template. The RNA may be mRNA, tRNA, rRNA, or another form of RNA, such as viral RNA. The cDNA may be single-stranded, double-stranded or may be hydrogen-bonded to a complementary RNA molecule as in an RNA/cDNA hybrid. Such a hybrid molecule would result from, for example, reverse transcription of an RNA template using a DNA polymerase.

The present invention solves the aforementioned problem by providing for a polymerase comprising,
a. an N-terminal 5′-3′ nuclease domain,
   i. stemming from Taq polymerase or,
   ii. a polymerase sharing at least 95% amino acid sequence identity with the N-terminal 5′-3′ exonuclease domain of Taq polymerase,
b. an adjacent and linked polymerase domain, stemming from a viral family A polymerase, wherein the polymerase domain stems preferably from,
   1. JGI20132J14458_100001622 (1607 amino acids), or a functional fragment that shares at least 98% amino acid sequence identity thereto, and is altered to comprise the following amino acid changes, Q627N, H751Q, Q752K, and V753K, or
   2. Ga0186926_122605 (1595 amino acids), or a functional fragment that shares at least 98% amino acid sequence identity thereto, and is altered to comprise the following amino acid changes, Q627N, H752Q, Q753K, and V754K, or
   3. Ga0080008_15802729 (1619 amino acids) or a functional fragment that shares at least 98% amino acid sequence identity thereto, and is altered to comprise the following amino acid changes, Q628N, H752Q, Q753K, and L754K, or
   4. Ga0079997_11796739 (1608 amino acids), or a functional fragment that shares at least 98% amino acid sequence identity thereto and is altered to comprise the following amino acid changes, Q627N, H752Q, Q753K, and I754K.

The 5′-3′ nuclease domain may be from Taq.

Taq is commercially available as a recombinant product or purified as native Taq from Thermus aquaticus (Perkin Elmer-Cetus). Recombinant Taq is designated as rTaq and native Taq is designated as nTaq. Native Taq is purified from T. aquaticus.

The 5′-3′ nuclease domain may also be from Tth purified from T. thermophilus or recombinant Tth.

Other thermostable polymerases that have been reported in the literature will also find use in the practice of the methods for making the 5′-3′ nuclease domain. Examples of these include polymerases extracted from the thermophilic bacteria Bacillus stearothermophilus, Thermus aquaticus, T. flavus, T. lacteus, T. rubens, T. ruber, and T. thermophilus.

Such polymerases are useful in PCR but also in RT-PCR. The present invention for the first time discloses a highly useful polymerase that can reverse transcribe RNA into DNA and react efficiently at high temperatures.

The activity of the polymerases of the invention do not require the presence of manganese so that the polymerases of the inventions may be used in conventional magnesium containing buffers. This compatibility with magnesium provides practical advantages in simplicity of reaction formulation and accuracy of synthesis, as is known in the art.

Preferably, in the polymerase according to the invention there is a peptide linker between the exonuclease domain and the polymerase domain and, optionally said peptide linker has the amino acid sequence according to SEQ ID NO. 19 (GGGGSGGGGS). In general, suitable linkers may be amino acid linkers comprising 5-15 amino acids, more preferably 7-12 amino acids, most preferably 9-11 amino acids. Alternatively, suitable linkers may be non-amino acid linkers.

Preferably, the polymerase domain is derived from a thermophilic viral family A polymerase. Other suitable polymerases include bacterial family A and non-thermophilic viral family A polymerases.

Preferably the exodomain of such a polymerase domain is inactivated. The 3′-5′ exonuclease (proofreading) activity was inactivated with a E to A mutation at residue 40 or 41 of the truncated enzyme. These would preferably be OS-1622 (577 amino acids), OP-2605 (578 amino acids), CS-2729 (578 amino acids) and PS-6739 (578 amino acids).

In some embodiments, the mutant ezmye claimed herein demonstrate increased reverse transcriptase activity that is at least 10% (e.g., 10%, 25%, 50%, 75%, 80%, 90%, 100%, 200%, etc.) more than wild type reverse transcriptase activity. In some embodiments, the mutant enzyme possess reverse transcriptase activity after 5 minutes at 60° C. that is at least 25% (e.g., 50%, 100%, 200%, etc.) of the reverse transcriptase activity of wild type reverse transcriptase after 5 minutes at 37° C. In some embodiments, the mutant reverse transcriptases, demonstrate one or more of the following properties: increased thermostability; increased thermoreactivity; increased resistance to reverse transcriptase inhibitors; increased ability to reverse transcribe difficult templates, increased speed/processivity; and increased specificity (e.g., decreased primer-less reverse transcription).

A native proofreading activity is inherent to the parent molecules used to derive the enzymes of this invention. To limit complications from this secondary activity such as degradation of primers, this proofreading exonuclease activity was disabled by mutagenesis in versions of the enzyme of this invention that are intended for analytic uses. Since this activity is beneficial in preparative use, this proofreading activity could be reconstituted by reversion of the proofreading exonuclease domain to the wild-type sequence, allowing the polymerase to excise mismatched bases and then insert the correctly matched base. A proofreading function coupled to high efficiency reverse transcription and inhibitor tolerance would enable high fidelity cDNA synthesis for improvements in applications such as RNA-seq and high accuracy RT-PCR.

Preferably, the polymerase domain is codon optimized for expression in E. coli. The purpose is to:
Rebalance codon usage
Decrease sequence complexity
Avoid rare codons

Most preferably, the polymerase is selected from the group of,
a. a polymerase (O15) as encoded by a nucleic acid according to SEQ ID NO. 9 or a nucleic acid that is at least 98% identical thereto,
b. a polymerase (O15) with the amino acid sequence according to SEQ ID NO: 10 or a polymerase that is at least 90% identical thereto,
c. a polymerase (O57) as encoded by a nucleic acid according to SEQ ID NO. 11 or a nucleic acid that is at least 98% identical thereto,
d. a polymerase (O57) with the amino acid sequence according to SEQ ID NO: 12 or a polymerase that is at least 90% identical thereto,
e. A polymerase (O58) as encoded by a nucleic acid according to SEQ ID NO. 13 or a nucleic acid that is at least 98% identical thereto, and
f. A polymerase (O58) with the amino acid sequence according to SEQ ID NO: 14 or a polymerase that is at least 90% identical thereto.

The invention also relates to certain polymerase domains an their uses:
OS-1622 (576 amino acids) SEQ ID NO. 24 is derived from Locus tag JGI20132J14458 100001622
OP-2605 (577 amino acids) SEQ ID NO. 25 is derived from Locus tag Ga0186926_122605
CS-2729 (577 amino acids) SEQ ID NO. 26 is derived from Locus tag Ga0080008 15802729
PS-6739 (577 amino acids) SEQ ID NO. 27 is derived from Locus tag Ga0079997 11796739

The invention relates therefore to a polymerase domain selected from the group of:
(a) OS-1622 (576 amino acids) SEQ ID NO. 24 is derived from Locus tag JGI20132J14458_100001622,
(b) OP-2605 (577 amino acids) SEQ ID NO. 25 is derived from Locus tag Ga0186926_122605,
(c) CS-2729 (577 amino acids) SEQ ID NO. 26 is derived from Locus tag Ga0080008_15802729, or
(d) PS-6739 (577 amino acids) SEQ ID NO. 27 is derived from Locus tag Ga0079997_11796739, or any polypeptide or functional fragment that shares more than 80%, 85%, 90%, 95% or 99% sequence identity with one of the above.

The invention relates to the use of such a polymerase domain for constructing a chimeric enzyme, preferably and enzyme with polymerase activity, more preferably with reverse transcriptase activity.

The invention relates to the use of one of the following metagenomic amino acid sequences for isolating a polmerase domain:
Locus tag JGI20132J14458_100001622 (1607 amino acids) SEQ ID NO. 20
Locus tag Ga0186926_122605 (1595 amino acids) SEQ ID NO. 21
Locus tag Ga0080008_15802729 (1619 amino acids) SEQ ID NO. 22
Locus tag Ga0079997_11796739 (1608 amino acids) SEQ ID NO. 23

Preferably, the invention relates also to the use of the regions (SEQ ID NOs. 20 to 23) and those that are 80%, 85%, 90% or more than 95% similar to these regions, for isolating a polymerase domain.

Thus, the present invention provides for also a polymerase comprising,
a. a polymerase domain, or functional fragment thereof with reverse transcriptase activity, stemming from a viral family A polymerase, wherein the polymerase domain stems preferably from,
   1. OS-1622 (SEQ ID NO. 24), defined herein as a 576 amino acid region from amino acid positions 1032 to 1607 of the polyprotein reported in the Integrated Microbial Genomes & Microbiomes database (IMG/M: https//img.jgi.doe.gov/m) as Locus ID:JGI20132J14458_100001622, or a functional fragment that shares at least 95% amino acid sequence identity thereto, or
   2. OP-2605 (SEQ ID NO. 25) defined herein as a 577 amino acid region from amino acid positions 1019 to 1595 of the polyprotein reported in the IMG/M database as Locus ID: Ga0186926_122605, or a functional fragment that shares at least 95% amino acid sequence identity thereto, or
   3. CS-2729 (SEQ ID NO. 26) defined herein as a 577 amino acid region from amino acid positions 1043 to 1619 of the polyprotein reported in the IMG/M database as Locus ID Ga0080008_15802729, or a functional fragment that shares at least 95% amino acid sequence identity thereto, or
   4. PS-6739 (SEQ ID NO. 27), defined herein as a 577 amino acid region from amino acid positions 1032 to 1608 of the polyprotein reported in the IMG/M database as Locus ID: Ga0079997_11796739, or a functional fragment that shares at least 95% amino acid sequence identity thereto.
b. an adjacent and linked domain from the RNase H-like, or RNase H superfamily that stems preferably from a N-terminal 5′-3 nuclease domain,
   i. stemming from Taq polymerase or,
   ii. a polymerase sharing at least 95% amino acid sequence identity with the N-terminal 5′-3′ nuclease domain of Taq polymerase,
c. amino acid alterations that comprise the following amino acid changes:
   1. OS-1622 Taq nuclease domain fusion (with mutations) (SEQ ID NO. 5) Q627N, H751Q, Q752K, and V753K
   2. OP-2605 Taq nuclease domain fusion (with mutations) (SEQ ID NO. 6) Q627N, H752Q, Q753K, and V754K
   3. CS-2729 Taq nuclease domain fusion (with mutations) (SEQ ID NO. 7) Q628N, H752Q, Q753K, and L754K
   4. PS-6739 Taq nuclease domain fusion (with mutations) (SEQ ID NO. 8) Q627N, H752Q, Q753K, and I754K.

The invention relates to a polymerase comprising,
a. the amino acid sequence of
   i. SEQ ID NO. 15 (OS-1622-Taq-wt) comprising the following additional amino acid changes, Q627N, H751Q, Q752K, and V753K,
   ii. or an amino acid sequence at least 90%, preferably at least 95%, more preferably at least 98% identical thereto,
b. the amino acid sequence of
   i. SEQ ID NO. 16 (OP-2605-Taq-wt) comprising the following additional amino acid changes, Q627N, H752Q, Q753K, and V754K,
   ii. or an amino acid sequence at least 90%, preferably at least 95%, more preferably at least 98% identical thereto,
c. the amino acid sequence of
   i. SEQ ID NO. 17 (CS-2729-Taq-wt) comprising the following additional amino acid changes, Q628N, H752Q, Q753K, and L754K, or an amino acid sequence at least 90%, preferably at least 95%, more preferably at least 98% identical thereto, or
d. the amino acid sequence of
   i. SEQ ID NO. 18 (PS-6739-Taq-wt) comprising the following additional amino acid changes, Q627N, H752Q, Q753K, and I754K,
   ii. or an amino acid sequence at least 90%, preferably at least 95%, more preferably at least 98% identical thereto.

The invention also relates to a method for amplifying template nucleic acids comprising contacting the template nucleic acids with a polymerase according to the invention, preferably wherein the method is reverse transcription PCR (RT-PCR).

Template nucleic acids according to the present invention may be any type of nucleic acids, such as RNA, DNA, or RNA:DNA hybrids. Template nucleic acids may either be artificially produced (e.g. by molecular or enzymatic manipulations or by synthesis) or may be a naturally occurring DNA or RNA. In some preferred embodiments, the template nucleic acids are RNA sequences, such as transcription products, RNA viruses, or rRNA. Advantageously, the method of the invention also enables amplification and detection/quantification of template nucleic acids, such as specific RNA target sequences, out of a complex mixture of target and non-target background RNA. For instance, the method of the invention allows amplification of an mRNA transcript from total human RNA or amplification of rRNA directly from bacterial cell lysate. In some embodiments, the method referred to herein is RT-PCR. RT-PCR may be quantitative RT-PCR (RT-qPCR), real-time RT-PCR, digital RT-PCR (dRT-PCR) or digital droplet RT-PCR (ddRT-PCR). In other embodiments, the method referred to herein is a method of amplifying RNA without high temperature thermal cycling, such as loop-mediated isothermal amplification (LAMP), helicase dependent amplification (HDA) and recombinase polymerase amplification (RPA).

In some embodiments, the method of the invention further comprises detecting and/or quantifying the amplified nucleic acids. Quantification/detection of amplified nucleic acids may be performed, e.g., using non-sequence-specific fluorescent dyes (e.g., SYBR® Green, EvaGreen®) that intercalate into double-stranded DNA molecules in a sequence non-specific manner, or sequence-specific DNA probes (e.g., oligonucleotides labelled with fluorescent reporters) that permit detection only after hybridization with the DNA targets, synthesis-dependent hydrolysis or after incorporation into PCR products.

In other particularly preferred embodiments, the generation of cDNA in step a) and the amplification of the generated cDNA in step b) are performed at isothermal conditions. Suitable temperatures may, for instance, be between 30-96° C., preferably 55-95° C., more preferably 55-75° C., most preferably 55-65° C.

In some embodiments, in the method of the invention, a polypeptide of the invention is used in combination with Taq DNA polymerase. In other embodiments, human serum albumin is added during amplification, preferably at a concentration of 1 mg/ml.

Preferably, the method comprises:
a) generating cDNA using a polypeptide according to any one of claims 1 to 6, and
b) amplifying the generated cDNA using a polypeptide according to any one of claims 1 to 6.

In some embodiments additional enzymes may be present in the reaction. These may be other polymerases, kinases, ligases, glycosylases, single-stranded binding proteins, RNase inhibitors, uracil-DNA glycosylases or the like.

The invention also relates to a kit comprising a polymerase according to the invention. In some embodiments, the invention relates to kits for amplifying template nucleic acids, wherein the kit comprises a polypeptide of the invention and a buffer. Optionally, the kit additionally comprises a DNA polymerase, oligonucleotide primers, salt solutions, buffer, or other additives. Buffers comprised in the kit may be conventional buffers containing magnesium. Suitable buffer solutions do not need to contain manganese.

As used herein, mutants, variants and derivatives refer to all permutations of a chemical species, which may exist or be produced, that still retain the definitive chemical activity of that chemical species. Examples include, but are not limited to compounds that may be detectably labelled or otherwise modified, thus altering the compound's chemical or physical characteristics.

In a preferred embodiment, the nucleic acid polymerase may be a DNA polymerase. The DNA polymerase may be any polymerase capable of replicating a DNA molecule. Preferably, the DNA polymerase is a thermostable polymerase useful in PCR. More preferably, the DNA polymerase is Taq, Tbr, Tth, Tih, Tfi, Tfl, Pwo, Kod, VENT, DEEPVENT, Tma, Tne, Bst, Pho, Sac, Sso, Poc, Pab, ES4 or mutants, variants and derivatives thereof having DNA polymerase activity.

Oligonucleotide primers may be any oligonucleotide of two or more nucleotides in length. Primers may be random primers, homopolymers, or primers specific to a target RNA template, e.g. a sequence specific primer.

Additional compositional embodiments comprise an anionic polymer and other reaction mixture components such as one or more nucleotides or derivatives thereof. Preferably the nucleotide is a deoxynucleotide triphosphate, dNTP, e.g. dATP, dCTP, dGTP, dTTP, dITP, dUTP,.alpha.-thio-dTNP, biotin-dUTP, fluorescein-dUTP, digoxigenin-dUTP.

Buffering agents, salt solutions and other additives of the present invention comprise those solutions useful in RT-PCR. Preferred buffering agents include e.g. TRIS, TRICINE, BIS-TRICINE, HEPES, MOPS, TES, TAPS, PIPES, CAPS. Preferred salt solutions include e.g. potassium chloride, potassium acetate, potassium sulphate, ammonium sulphate, ammonium chloride, ammonium acetate, magnesium chloride, magnesium acetate, magnesium sulphate, manganese chloride, manganese acetate, manganese sulphate, sodium chloride, sodium acetate, lithium chloride, and lithium acetate. Preferred additives include e.g. DMSO, glycerol, formamide, betain, tetramethylammonium chloride, PEG, Tween 20, NP 40, extoine, polyols, E. coli SSB protein, Phage T4 gene 32 protein, and serum albumin. Additional compositional embodiments comprise other components that have been shown to reduce the inhibitory effect of reverse transcriptase on DNA polymerase, e.g. homopolymeric nucleic acids as described in EP 1050587 B1.

Further embodiments of this invention relate to methods for generating nucleic acids from an RNA template and further nucleic acid replication. The method comprises: a) adding an RNA template to a reaction mixture comprising at least one reverse transcriptase and/or mutants, variants and derivatives thereof and at least one nucleic acid polymerase, and/or mutants, variants and derivatives thereof, and an anionic polymer that is not a nucleic acid, and one or more oligonucleotide primers, and b) incubating the reaction mixture under conditions sufficient to allow polymerization of a nucleic acid molecule complementary to a portion of the RNA template. In a preferred embodiment the method includes replication of the DNA molecule complementary to at least a portion of the RNA template. More preferably the method of DNA replication is polymerase chain reaction (PCR). Most preferably the method comprises coupled reverse transcriptase-polymerase chain reaction (RT-PCR).

The invention also relates to a vector encoding a polymerase according to the invention.

Preferably the vector is in a transformed host cell.

In some embodiment the invention relates to a viral family A polymerase, or a portion thereof comprising one of the following mutations/alterations, i.e. is an altered enzyme, selected from the group of.
a. Q627N or Q628N
b. H751Q or H752Q
c. Q752K or Q753K
d. V753K or V754K or L754K or I754K
or mutations in similar residues from locally aligned family A polymerases per the amino acid numbering of the Taq nuclease domain-linked polymerases as outlined above.

Herein, “altered polymerase enzyme” means that the polymerase has at least one amino acid change compared to the control polymerase enzyme, for example the family A polymerase. In general, this change will comprise the substitution of at least one amino acid for another. In certain instances, these changes will be conservative changes, to maintain the overall charge distribution of the protein. However, the invention is not limited to only conservative substitutions. Non-conservative substitutions are also envisaged in the present invention. Moreover, it is within the contemplation of the present invention that the modification in the polymerase sequence may be a deletion or addition of one or more amino acids from or to the protein, provided that the polymerase has improved activity (over e.g. the wildtype) with respect to reverse transcriptase activity, thermostability or inhibitor resistance as compared to a control polymerase enzyme, such as the wild type.

The altered polymerase will generally and preferably be an “isolated” or “purified” polypeptide. By “isolated polypeptide” a polypeptide that is essentially free from contaminating cellular components is meant, such as carbohydrates, lipids, nucleic acids or other proteinaceous impurities which may be associated with the polypeptide in nature. One may use a His-tag for purification, but other means may also be used. Preferably, at least the altered polymerase may be a “recombinant” polypeptide.

In these embodiments the ideal reaction is only reverse transcription and/or RT-PCR. Preferably it is reverse transcription.

The present invention solves the aforementioned problem by providing for a method of making a polymerase comprising,
i) isolating an N-terminal 5′-3′ nuclease domain, stemming from Taq polymerase or, a polymerase sharing at least 95% amino acid sequence identity with the N-terminal 5′-3′ nuclease domain of Taq polymerase,
ii) linking thereto a polymerase domain, stemming from a viral family A polymerase, wherein the polymerase domain stems preferably from,
   1. JGI20132J14458_100001622 (1607 amino acids), or a functional fragment that shares at least 98% amino acid sequence identity thereto, and is altered to comprise the following amino acid changes, Q627N, H751Q, Q752K, and V753K, or
   2. Ga0186926_122605 (1595 amino acids), or a functional fragment that shares at least 98% amino acid sequence identity thereto, and is altered to comprise the following amino acid changes, Q627N, H752Q, Q753K, and V754K, or
   3. Ga0080008_15802729 (1619 amino acids) or a functional fragment that shares at least 98% amino acid sequence identity thereto, and is altered to comprise the following amino acid changes, Q628N, H752Q, Q753K, and L754K, or
   4. Ga0079997_11796739 (1608 amino acids), or a functional fragment that shares at least 98% amino acid sequence identity thereto and is altered to comprise the following amino acid changes, Q627N, H752Q, Q753K, and I754K.

In one embodiment the polymerase consists of only the viral family A polymerase domain and the mutations mentioned above.

The invention relates to a method for amplifying a target RNA molecular suspected of being present in a sample, the method comprising the steps of:
(a) treating said sample with a first primer, which primer is sufficiently complementary to said target RNA to hybridize therewith, and a thermostable DNA polymerase according to the invention having the claimed reverse transcriptase activity in the presence of all four deoxyribonucleoside triphosphates, in an appropriate buffer and at a temperature sufficient for said first primer to hybridize with said target RNA and said thermostable DNA polymerase to catalyze to polymerization of said deoxyribonucleoside triphosphates to provide cDNA complementary to said target RNA;
(b) treating said cDNA formed in step (a) to provide single-stranded cDNA:
(c) treating said single-stranded cDNA formed in step (b) with a second primer, wherein said second primer can hybridize to said single-stranded cDNA molecule and initiate synthesis of an extension product in the presence of a the same polymerase according to the invention or another thermostable polymerase under appropriate conditions to produce a double-stranded cDNA molecule; and
(d) amplifying the double-stranded cDNA molecule of step (c) by a polymerase chain reaction.

Ideally, said RNA target is diagnostic of a genetic or infectious disease.

The invention relates to a method for preparing duplex cDNA from an RNA template that comprises the steps of:
(a) treating said RNA template with a first primer, which primer is sufficiently complementary to said RNA template to hybridize therewith, and a thermostable DNA polymerase according to the invention having reverse transcriptase activity in the presence of all four deoxyribonucleoside triphosphates, in an appropriate buffer and at a temperature sufficient for said first primer to hybridize with said RNA template and said thermostable DNA polymerase to catalyze the polymerization of said deoxyribonucleoside triphosphates to provide cDNA complementary to said target RNA; optionally
(b) treating said cDNA formed in step (a) to provide single-stranded cDNA;
(c) treating said single-stranded cDNA formed in step (b) with a second primer, wherein said second primer can hybridize to said single-stranded cDNA molecule and initiate synthesis of an extension product in the presence of said same polymerase or another thermostable polymerase under appropriate conditions to produce a double-stranded cDNA molecule.

Preferably the 3′-5′ proofreading exonuclease activity of the polymerase is inactivated. In many analytical applications the 3′-5′ proofreading exonuclease activity of the polymerase is not critical; however, there are applications for which it can be advantageous for the 3′-5′ proofreading activity to be active, allowing for high-fidelity cDNA synthesis. Hence, in some embodiments the 3′-5′ proofreading exonuclease activity is present.

The primer typically contains 10-30 nucleotides, although that exact number is not critical to the successful application of the method. Short primer molecules generally require cooler temperatures to form sufficiently stable hybrid complexes with the template.

The present methods provide that the reverse transcription of the annealed primer-RNA template is catalyzed by the claimed polymerase, i.e. a thermostable polymerase according to the invention. As used herein, the term “thermostable polymerase” refers to an enzyme that is heat stable or heat resistant and catalyzes polymerization of deoxyribonucleotides to form primer extension products that are complementary to a nucleic acid strand. Thermostable polymerases useful herein are not irreversibly inactivated when subjected to elevated temperatures for the time necessary to effect destabilization of single-stranded nucleic acids.

The thermostable polymerases described herein are significantly more thermostable than commonly used retroviral RTs and are active at commonly used PCR extension temperatures at which single-stranded secondary structures would be destabilized.

Irreversible denaturation of the enzyme refers to substantial loss of enzyme activity. Preferably a thermostable DNA polymerase will not irreversibly denature at about 65°-75° C. under polymerization conditions.

Of course, it will be recognized that for the reverse transcription of mRNA, the template molecule is single-stranded and therefore, a high temperature denaturation step is unnecessary.

But high temperature reverse transcription is advantageous for reducing secondary structure in single-stranded mRNA molecules, potentially improving cDNA yield.

A first cycle of primer elongation provides a double-stranded template suitable for denaturation and amplification as referred to above.

The heating conditions will depend on the buffer, salt concentration, and nucleic acids being denatured. Temperatures for RNA destabilization typically range from 50°-80° C. for a time sufficient for denaturation to occur which depend on the nucleic acid length, base content, and complementarity between single-strand sequences present in the sample, but typically about 0.5 to 4 minutes.

The thermostable enzyme preferably has optimum activity at a temperature higher than about 40° C., e.g., 65°-75° C. At temperatures much above 42° C., DNA and RNA dependent polymerases, other than thermostable DNA polymerases, are inactivated. Thus, they are inappropriate for catalyzing high temperature polymerization reactions utilizing a DNA or RNA template. Previous RNA amplification methods require incubation of the RNA/primer mixture in the presence of reverse transcriptase at a 37°-42° C. prior to the initiation of an amplification reaction.

Hybridization of primer to template depends on salt concentration and composition and length of primer. Hybridization can occur at higher temperatures (e.g., 45°-70° C.), which are preferred when using a thermostable polymerase. Higher temperature optimums for the thermostable enzyme enable RNA transcription and subsequent amplification to proceed with greater specificity due to the selectively of the primer hybridization process. Preferably, the optimum temperature for reverse transcription of RNA ranges from about 55°-75° C., more preferably 65°-70° C.

The methods provided have numerous applications, particularly in the field of molecular biology and medical diagnostics. The reverse transcriptase activity described provides a cDNA transcript from an RNA template. The methods provide production and amplification of DNA segments from an RNA molecule, wherein the RNA molecule is a member of a population of total RNA or is present in a small amount in a biological sample. Detection of a specific RNA molecule present in a sample is greatly facilitated by a thermostable DNA polymerase used in the methods described herein. A specific RNA molecule or a total population of RNA molecules can be amplified, quantitated, isolated, and, if desired, cloned and sequenced using a thermostable DNA polymerase as described herein.

The methods and compositions of the present invention are a vast improvement over prior methods of reverse transcribing RNA into a DNA product. These methods provide products for PCR amplification or perform the PCR directly in one tube. The invention provides more specific and, therefore, more accurate means for detection and characterization of specific ribonucleic acid sequences, such as those associated with infectious diseases, genetic disorders, or cellular disorders.

### EXAMPLES

### Example 1

### Domain Structure of the Full Viral Polyprotein

Four previously uncharacterized viral metagenomic gene product candidates were identified from the Joint Genome Institute Integrated Microbial Genomes and Microbiomes system as multidomain polyproteins.
Locus tag JGI20132J14458_100001622 (1607 amino acids) SEQ ID NO. 20
Locus tag Ga0186926_122605 (1595 amino acids) SEQ ID NO. 21
Locus tag Ga0080008_15802729 (1619 amino acids) SEQ ID NO. 22
Locus tag Ga0079997_11796739 (1608 amino acids) SEQ ID NO. 23

These were chosen by the inventors based on careful analysis including selection criteria as, (i) sampling location in environments in which thermophilic organisms would be expected to grow and (ii) the finding that regions of the polyprotein display protein family homology to known DNA polymerase family A proteins as determined using the Pfam database (Nucleic Acids Research (2019) doi: 10.1093/nar/gky995). The Pfam database is a large collection of protein families represented by multiple sequence alignments and hidden Markov models. Although the analysis of each of the full protein sequences revealed a large uncharacterized region at the N-terminal portion of the putative protein with a domain of unknown function, each also contained domains at the C-termal portion with homology to DNA polymerase family A proteins and an associated domain with homology to Pol A 3′-5′ proofreading exonuclease domains. This suggested to the inventors that these proteins may function in viral nucleic acid replication or repair and may possess thermoactive DNA polymerase and/or reverse transcriptase activities.

### Truncation and Protein Engineering

We next sought to isolate an active polymerase region from the large putative viral protein by truncating the full protein according to the predicted Pfam structural and functional information.

The core polymerase sequences we isolated are as follows:
OS-1622 (576 amino acids) SEQ ID NO. 24 is derived from Locus tag JGI20132J14458_100001622
OP-2605 (577 amino acids) SEQ ID NO. 25 is derived from Locus tag Ga0186926_122605
CS-2729 (577 amino acids) SEQ ID NO. 26 is derived from Locus tag Ga0080008_15802729
PS-6739 (577 amino acids) SEQ ID NO. 27 is derived from Locus tag Ga0079997_11796739

Each of the candidate viral polymerase DNA sequences was codon optimized for expression in E. coli, and the corresponding synthetic gene fragments were constructed and assembled into an expression vector. Compared with the predicted wild-type amino acid sequence obtained from the previously identified viral genes, each polymerase was engineered in two ways: Fusion with the Taq DNA polymerase 5′-3′ nuclease domain via an intervening eight amino acid flexible linker with the sequence GGGGSGGGGS and incorporation of four mutations in regions of the polymerase predicted to associate with template nucleic acid (FIG. 1).

In addition, the 3′-5′ exonuclease (proofreading) activity was inactivated with a E to A mutation at residue 40 or 41 of the truncated enzyme.

The viral polymerase domain was fused at the N-terminus with the 5′-3′ nuclease domain of Taq polymerase via a flexible linker.

The Taq fusions were then mutated as follows:
OS-1622-Taq-wt (Q627N, H751Q, Q752K, V753K)
OP-2605-Taq-wt (Q627N, H752Q, Q753K, V754K)
CS-2729-Taq-wt (Q628N, H752Q, Q753K, L754K)
PS-6739-Taq-wt (Q627N, H752Q, Q753K, I754K)

The OP-2605-Taq-mut sequence was then further altered by incorporating seven stabilizing mutations as described below.

### Example 2

Using sequence divergent thermostable viral family A DNA polymerases identified from hot spring metagenomic sampling studies (see above), we show that the combination of two protein engineering steps induced robust, high activity, inhibitor resistant reverse transcription activity to the DNA polymerases in PCR-based RNA detection assays. The two modifications to the wild-type sequences were the N-terminal Taq nuclease fusion and the incorporation of four mutations in regions of the polymerase predicted to associate with template nucleic acid. Based on these findings, this protein engineering methodology may be generally applicable to improving on basal reverse transcription activity in a broad set of viral family A DNA polymerases.

The viral family A polymerases were selected from a database containing sequences from metagenomic sampling studies, the Joint Genome Institute Integrated Microbial Genomes and Microbiomes system (https://img.jgi.doe.gov/). Based on sampling locations in hot spring regions of Yellowstone National Park and similarity to known viral family A polymerases, a number of orthologs were selected (Table 1).

The C-terminal 576 or 577 amino acids of the larger putative viral gene corresponded to the polymerase domain and showed significant divergence from the gene shuffled M160 viral family A variant (WO 2019/211749), with amino acid identity ranging from 79 to 85 percent. In addition, these additional viral family A polymerases show divergence from each other, with pairwise amino acid percent identity ranging from 79 to 89 percent.

Each of the candidate viral polymerase DNA sequences was codon optimized for expression in E. coli, and the corresponding synthetic gene fragments were constructed and assembled into an expression vector. Compared with the predicted wild-type amino acid sequence obtained from the previously identified viral genes, each polymerase was engineered in two ways: Fusion with the Taq DNA polymerase 5′-3′ nuclease domain via an intervening eight amino acid flexible linker with the sequence GGGGSGGGGS and incorporation of four mutations in regions of the polymerase predicted to associate with template nucleic acid (FIG. 1). After verification of the sequences of each of the nucleic acid constructs (SEQ ID NO 1-4), the engineered polymerases (SEQ ID NO 5-8) were overexpressed in BL21 cells. Overexpressed protein was not detected for CS-2729, but for the other three polymerases, soluble protein was produced, and stability was maintained after heating of lysate at 75° C. for 10 minutes to precipitate host E. coli protein and centrifugation to clarify lysate. Reverse transciptase activity was tested from lysates in RT-qPCR reactions (20 μl) containing Taq polymerase and Eva Green dye, targeting a 243-nucleotide region of the MS2 RNA genome (FIG. 2). Incubation was at 70° C. for 1 min; followed by 94° C. for 30 s; followed by 40 cycles of 94° C. for 5 s and 70° C. for 20 s with fluorescence data collection during the anneal/extension step. Compared with the engineered, gene shuffled M503 polymerase (WO 2019/211749), the amplification fluorescence curves of the additional engineered viral family A polymerases were very similar, indicating highly efficient reverse transcriptase activity for all polymerases at the 70° C. reaction temperature in just one minute. In contrast, in reactions without reverse transcriptase-containing lysate and containing Taq polymerase only, amplification from the RNA template was late and inefficient as expected.

Whereas each engineered viral family A polymerase was stable in cell lysate after incubation at 75° C. for 10 minutes, some activity loss was observed after incubation at 80° C. for 5 minutes in reaction buffer. In order to improve the thermal stability of the engineered OP-2605 polymerase, seven amino acid positions were identified for combinatorial mutagenesis and variant screening for elevated reverse transcriptase activity after an 80° C. incubation. With a homology model of the OP-2605 polymerase using a well-studied KlenTaq structure as a template, thirteen stabilizing point mutations in total were predicted among the seven amino acid positions based on local amino acid environment. A variant mutant library was constructed in which each of the 48 possible combinations of these thirteen mutations could be tested at random. After screening a total of 64 E. coli lysates overexpressing the OP-2605 variants, it was found that 49 of these (76.6%) did not maintain efficient reverse transcriptase activity at 70° C. and so were discarded. The remaining 15 variants were tested for reverse transcriptase activity after incubation at 80° C. for 5 minutes (FIG. 3). RT-qPCR reactions (20 μl) containing Taq polymerase and Eva Green dye targeted a 243 nucleotide region of the MS2 RNA genome. Incubation was at 70° C. for l min; followed by 94° C. for 30 s; followed by 40 cycles of 94° C. for 5 s and 70° C. for 20 s with fluorescence data collection during the anneal/extension step. It was found that three engineered OP-2605 variants showed improved thermal stability as measured by the lower Cq values after heat treatment compared with the parental polymerase, indicating that they retained higher activity levels. The mutations introduced in the three improved variants identified from the mutant library screening are shown in Table 2.

For further analysis of the enzymes, the three high activity engineered OP-2605 variants were then expressed in E. coli and purified by strong cation exchange and heparin spin-column chromatography as is known in the art. DNA polymerization activities of the variants were measured by determining the relative rates of nucleotide incorporation (FIG. 4) using a primed M13 template. Reactions (20 μl) contained 20 mM Tris, pH 8.8, 10 mM (NH4)2SO4, 10 mM KCl, 2 mM MgSO4, 0.1% Triton X-100, 200 μM dNTPs, 1X SYBR Green I (Thermo Fisher), 7.5 pg/ml M13mp18 DNA, 0.25 mM each of a mixture of three primers 24-33 nt in size, and 0.1-1 ng of polymerase. Reactions were incubated at the indicated temperatures, fluorescence readings were taken every 15 seconds, and fluorescence initial slope values were calculated and compared. The temperature at which the activity was highest was set at 1 and other values were plotted relative to this number. As shown in FIG. 4, each of the O15, O57, and O58 variants display peak activity from 65-70° C.

To test the sensitivity of O15, O57, and O58 in detection of viral MS2 RNA, RT-qPCR reactions were performed using a dual-quenched FAM-labeled hydrolysis probe for amplification detection (FIG. 5). Reactions (20 μl) contained Taq polymerase and targeted a 243-nucleotide region of the MS2 RNA genome. Incubation was at 70° C. for 1 min; followed by 94° C. for 30 s; followed by 40 cycles of 94° C. for 5 s and 70° C. for 20 s with fluorescence data collection during the anneal/extension step. It was found that all three of the engineered variants catalyzed high efficiency reverse transcription of the viral RNA in the 1-minute high temperature incubation step, supporting efficient and sensitive detection of the MS2 viral RNA. As few as 100 copes were detected, the smallest quantity tested, indicating a high degree of sensitivity and specificity.

The performance of nucleic acid amplification-based detection methods are often inhibited by the presence of inhibitors in target samples. One of these inhibitors, heparin, is commonly used as an anticoagulant and can copurify with nucleic acid samples derived from blood. To test the compatibility of the O15, O57, and O58 engineered variants with the detection of viral MS2 RNA in the presence of an inhibitor, RT-qPCR reactions were performed with increasing quantities of heparin and compared with the engineered, gene shuffled M503 polymerase (FIG. 6). Reactions (20 μl) contained Taq polymerase, 1×10⁶ copies of the MS2 RNA genome, and incubation was at 70° C. for 2 min; followed by 94° C. for 30 s; followed by 40 cycles of 94° C. for 5 s and 70° C. for 20 s. Of the three engineered variants, O57 displayed the greatest heparin resistance as indicated by the lowest Cq values at elevated heparin concentrations. In addition, the O57 variant displayed a significantly greater inhibitor resistance than the engineered, gene shuffled M503 polymerase, with Cq values 3.7-6.5 lower in the presence of greater than 1.25 ng/μl heparin.

Table 1 shows the identification of potential thermophilic viral Family A DNA polymerases.

Metagenomic viral family A polymerases were identified from Yellowstone hot spring sampling studies. The protein product size corresponding to the total size of the putative viral gene is indicated in addition to the size of the aligned polymerase domain. The percent identity is relative to the gene shuffled M160 polymerase variant.

TABLE 1

| | | | Total | Pol | Amino |
|---|---|---|---|---|---|
| | | | Size | Size | Acid |
| | Geographic | | (amino | (amino | Percent |
| Locus Tag | Location | Name | acids) | acids) | Identity |
| JGI20132J14458_ | Octopus Spring, | OS- | 1607 | 577 | 85 |
| 100001622 | Wyoming, US | 1622 | | | |
| Ga0186926_ | Obsidian Pool, | OP- | 1595 | 578 | 79 |
| 122605 | Wyoming, US | 2605 | | | |
| Ga0080008_ | Conch Spring, | CS- | 1619 | 578 | 84 |
| 15802728 | Wyoming, US | 2729 | | | |
| | Perpetual Spouter, | | | | |
| Ga0079997_ | Yellowstone Park, | PS- | 1608 | 578 | 84 |
| 11796739 | Wyoming, US | 6739 | | | |

Table 2 shows OP-2605 stabilizing mutant sequences.

TABLE 2

| | | Nucleic acid | Amino acid |
|---|---|---|---|
| Enzyme | Mutations | sequence | sequence |
| O15 | D592E, K644R, P665S, | SEQ ID NO 9 | SEQ ID NO 10 |
| | D687G, K716R, K743R, | | |
| | N772A | | |
| O57 | D592V, K644R, P665K, | SEQ ID NO 11 | SEQ ID NO 12 |
| | D687G, K716R, K743A, | | |
| | N772A | | |
| O58 | D592E, K644R, P665K, | SEQ ID NO 13 | SEQ ID NO 14 |
| | D687E, K716R, K743R, | | |
| | N772A | | |

Most astonishingly the new polymerases differ substantially from those previously developed; see WO 2019/211749 and EP1934339.

### Sequences Listing

Codon optimized OS-1622 Taq fusion DNA sequence
(with mutations) Length: 2,628, Type: DNA, Source: Synthetic
SEQ ID NO. 1
ATGCGTGGTATGCTTCCACTGTTTGAACCGAAAGGCCGTGTGCTGCTGGTTGAT
GGCCACCATCTGGCCTATCGTACCTTCCATGCGCTGAAAGGCCTGACGACCAG
CCGCGGCGAACCGGTGCAGGCGGTGTATGGCTTTGCGAAAAGCCTGCTGAAAG
CGCTGAAAGAAGATGGCGATGCGGTTATTGTGGTGTTTGATGCGAAAGCGCCG
AGCTTTCGTCATGAAGCGTATGGCGGCTATAAAGCGGGTCGTGCGCCGACCCC
GGAAGATTTTCCGCGTCAGCTGGCCCTGATTAAAGAACTGGTGGATCTGCTGG
GCCTGGCGCGTCTGGAAGTGCCGGGCTATGAAGCGGATGATGTGCTGGCCAGC
CTGGCCAAAAAAGCGGAAAAAGAAGGCTACGAAGTTCGTATTCTGACCGCCG
ATAAAGACCTGTATCAGCTGCTGTCTGATCGTATTCATGTGCTGCATCCTGAGG
GTTATCTGATTACCCCGGCGTGGCTGTGGGAAAAATATGGCCTGCGTCCGGAT
CAGTGGGCGGATTATCGTGCGCTGACCGGCGATGAAAGCGATAACCTGCCGGG
CGTGAAAGGCATTGGCGAAAAAACCGCGCGTAAACTGCTGGAAGAATGGGGC
AGCCTGGAAGCGCTGCTGAAAAACCTGGATCGTCTGAAACCGGCGATTCGTGA
AAAGATCTTAGCGCACATGGATGATCTGAAACTGAGCTGGGATCTGGCCAAAG
TGCGTACCGATCTGCCGCTGGAAGTGGATTTTGCGAAACGTCGTGAACCGGAT
CGTGAACGTCTGCGTGCGTTTCTGGAACGTCTGGAATTTGGCAGCCTGCTGCAT
GAATTTGGCCTGCTGGAAAGCGGTGGCGGCGGTTCTGGCGGTGGTGGCAGCAA
CATTCCCAAGCCGATCCTTAAACCACAACCTAAAGCACTTGTTGAACCGGTTCT
GTGCGACAGCGTCGATGAAATCCCCACAAAGTTTAACGAACCAATCTATTTCG
ATCTTGCAACCGACGGGGACCGCCCGGTGTTAGCATCCATCTACCAACCCCAC
TTTGAACGTAAGGTCTATTGTCTTAACTTATTAAAAGAGAAGCCTACTCGTTTT
AAGGAGTGGCTTCTGAAGTTCAGCGAGATTCGTGGCTGGGGTTTAGACTTCGA
TCTGCGCGCCTTAGGTTACACATACGAGCAGTTACGCGATAAAAAGATTGTGG
ACGTGCAGCTGGCTATCAAAGTCCAGCATCATGAACGCTTCAAGCAGAACGGT
ACTAAGGGTGAAGGCTTTCGTCTGGACGACGTGGCCCGCGATTTGTTAGGAAT
CGAGTACCCTATGGATAAGACCAAGATCCGCGAGACGTTTAAAAATAACATTT
TTCACTCATTTAGCAATGAGCAATTGTTGTATGCATCTCTTGACGCTTATATCC
CTCACCTGCTTTACGAACAATTAACGAGTTCAACGCTTAATTCGCTGGTTTACC
AGTTAGATCAGCAAGCACAGAAAATTGTGGTGGAAACAAGTCAGAATGGTAT
GCCGGTTAAATTAAAGGCTCTGGAAGAGGAAATCCATCGCTTGACGCAGCTTC
GTAACCAAATGCAAAAAGAAATTCCTTTTAACTACAATTCGCCTAAACAGACA
GCTAAATTCTTCCGTGTTGATTCCAGCAGTAAGGACGTTCTTATGGACCTGGCA
TTACAAGGTAATGAGATGGCGAAACGCGTTTTGGAAGCCCGCCAGGTCGAGAA
GAGCCTGGCCTTCGCTAAGGATCTTTATGACATCGCGAAACGCAGCGGAGGGC
GCGTTTATGGAAATTTCTTTACCACAACGGCGCCGAGTGGACGTATGAGTTGT
AGCGATATCAACCTTCAAAATATCCCTCGCCGCTTACGCCAATTCATTGGCTTT
GATACGGAAGATAAACGTCTTATTACGGCAGACTTTCCTCAAATCGAGCTGCG
CTTAGCGGGAGTCATCTGGAACGAGAGCGAGTTCATTGAAGCCTTTAAACAAG
GCATTGACCTTCATAAATTAACGGCGTCAATTCTGTTTGAGAAGAATATTGAG
GAAGTCGGGAAGGAGGAACGTCAGATTGGTAAATCGGCGAATTTTGGATTAAT
TTATGGAATTGCTCCTAAAGGTTTTGCTGAGTACTGTATTACGAACGGAATTAA
TATGACGGAAGAGCAGGCATACGAAATTGTACGCAAGTGGAAGAAATATTAT
ACTAAGATTGCGGAGCAGCAAAAAAAGGCTTATGAACGTTTCAAATATAACGA
GTACGTGGACAACGAAACATGGCTGAATCGCACCTACCGTGCATGGAAACCAC
AAGATTTGTTAAACTACCAGATCCAAGGATCTGGTGCTGAGTTGTTCAAGAAG
GCCATTGTCCTGCTGAAGGAGGCAAAACCGGATCTTAAGATCGTCAACTTGGT
ACACGATGAGATTGTTGTCGAGGCCGACTCTAAGGAAGCCCAAGACCTTGCCA
AGCTGATCAAAGAGAAGATGGAAGAAGCCTGGGACTGGTGTTTGGAAAAGGC
GGAGGAGTTCGGCAACCGTGTAGCCAAGATTAAACTTGAAGTAGAGCAGCCG
AACGTAGGGGATACATGGGAGAAATCG

Codon optimized OP-2605 Taq fusion DNA sequence
(with mutations) Length: 2,631, Type: DNA, Source: Synthetic
SEQ ID NO. 2
ATGCGTGGTATGCTTCCACTGTTTGAACCGAAAGGCCGTGTGCTGCTGGTTGAT
GGCCACCATCTGGCCTATCGTACCTTCCATGCGCTGAAAGGCCTGACGACCAG
CCGCGGCGAACCGGTGCAGGCGGTGTATGGCTTTGCGAAAAGCCTGCTGAAAG
CGCTGAAAGAAGATGGCGATGCGGTTATTGTGGTGTTTGATGCGAAAGCGCCG
AGCTTTCGTCATGAAGCGTATGGCGGCTATAAAGCGGGTCGTGCGCCGACCCC
GGAAGATTTTCCGCGTCAGCTGGCCCTGATTAAAGAACTGGTGGATCTGCTGG
GCCTGGCGCGTCTGGAAGTGCCGGGCTATGAAGCGGATGATGTGCTGGCCAGC
CTGGCCAAAAAAGCGGAAAAAGAAGGCTACGAAGTTCGTATTCTGACCGCCG
ATAAAGACCTGTATCAGCTGCTGTCTGATCGTATTCATGTGCTGCATCCTGAGG
GTTATCTGATTACCCCGGCGTGGCTGTGGGAAAAATATGGCCTGCGTCCGGAT
CAGTGGGCGGATTATCGTGCGCTGACCGGCGATGAAAGCGATAACCTGCCGGG
CGTGAAAGGCATTGGCGAAAAAACCGCGCGTAAACTGCTGGAAGAATGGGGC
AGCCTGGAAGCGCTGCTGAAAAACCTGGATCGTCTGAAACCGGCGATTCGTGA
AAAGATCTTAGCGCACATGGATGATCTGAAACTGAGCTGGGATCTGGCCAAAG
TGCGTACCGATCTGCCGCTGGAAGTGGATTTTGCGAAACGTCGTGAACCGGAT
CGTGAACGTCTGCGTGCGTTTCTGGAACGTCTGGAATTTGGCAGCCTGCTGCAT
GAATTTGGCCTGCTGGAAAGCGGTGGCGGCGGTTCTGGCGGTGGTGGCAGCAA
TACTACTACATTAAGTGTGAAGCAGGAGGTAAAATCCCTTGTTAAACCGGTAG
TGTGCGATTCGATTGATAAAATTCCAGCAAAGTTCGATGAACCCGTTTATTTTG
ATCTTGCTACCGACAATGACAAGCCTGTTTTGGCCTCTATCTATCAATCTCATT
TTGGACATGACGTCTACTGCTTGAACTTATTAAAGGAGAAACCAGCCCGCCTG
AAAGATTGGTTGTTGAAATTCAGCGAGATTCGTGGCTGGGGTTTAGATTATGA
CTTGCGCGTTCTTGGCTATACTTATGAACAACTTAAAGACAAAAAAATTGTAG
ACGTACAACTTGCTATTAAGGTGCAACACTACGAACGTTTTCGCCAGAACGGA
GCGAAGGGCGAGGGTTTCAAGCTTGACGATGTCGCCCGCGACCTGTTGGGAAT
CGAATACCCCATGGACAAGACGAAAATCCGTACTACCTTCAAGCAAAATATGT
ATAATTCTTTTAATAAAGACCAGTTATTGTATGCCAGCCTGGATGCTTACATCC
CTCACTTGCTTTACGAGCAACTGAGTTCAAATACTTTGAACAGTTTGGTCTATC
AGCTGGACCAGCAAGTTCAAAAGATCGGCATCGAGACGTCACAACATGGTCTT
CCTGTCCGTCTGCAAGCATTGCAAGAAGAGATTGATAAGTTATCACAGATCAA
GAAACGCATTCAGAAAGAGATCCCATTCAATTATAACTCCCCTAAACAAACCA
CCCAGTACTTGGGCATCGATAGCTCCAGTAAGGACGTGTTGATGGACCTGGCG
TTAAAGGGCAACGAGTTAGCTAAGAAAATCCTTGAGGCTCGTCAAATTGAAAA
GGCTCTGACCTTCGCTAAAGATTTATACGATTTGGCGAAGCGTAATAACGGAC
GTATTTACGGTAACTTCTTTACTACTACCGCGCCATCTGGGCGTATGTCGTGTA
GCGACATCAACTTGCAAAACATTCCACGCAAGTTGCGTCCGTTCATTGGCTTTG
AAACTGAAGATAAGAAACTGATTACCGCTGATTTTCCCCAAATCGAATTGCGC
TTGGCTGGTGTAATCTGGAACGAACCAAAGTTTATTGAAGCCTTCAATCAAGG
AATTGACTTACACAAGTTGACAGCATCAATTCTGTTCGATAAGCGCTCGGTCG
ATGAGGTCAGTAAAGAAGAGCGCCAGATCGGGAAGTCTGCAAACTTTGGGTTG
ATCTATGGGATCTCCCCGAAAGGATTCGCTGAGTACTGCATCACTAATGGAAT
CAACATGACCGAAGAGATCGCATACGAGATCGTCAAGAAGTGGAAAAAATAT
TATACAAAAATCACTGAACAACAAAAGAAGGCGTATGAACGCTTCAAATACG
GGGAGTACGTCGATAACGAAACCTGGTTAAATCGTACCTATCGTGCCTATAAA
CCCCAGGACTTGTTGAACTACCAGATCCAGGGTTCTGGGGCTGAGCTGTTCAA
AAAAGCTATCATCCTGTTGAAAGAGGAGGAGCCAAGTGTTAAAATTGTCAACT
TGGTCCATGATGAAATCGTTGTTGAGGCTGATAGTAAAGATGCTCAGGACGTA
GCCAATTTAATTAAAGAAAAGATGGGGCAGGCCTGGGATTACTGCTTGGATAA
GGCCAAAGAATTCGGAAACCGCGTAGCGGAAATTAAGCTTGAAGTAGAAGAG
CCCAATGTCAGTGAAGTTTGGGAAAAGGGC

Codon optimized CS-2729 Taq fusion DNA sequence
(with mutations) Length: 2,631, Type: DNA, Source: Synthetic
SEQ ID NO. 3
ATGCGTGGTATGCTTCCACTGTTTGAACCGAAAGGCCGTGTGCTGCTGGTTGAT
GGCCACCATCTGGCCTATCGTACCTTCCATGCGCTGAAAGGCCTGACGACCAG
CCGCGGCGAACCGGTGCAGGCGGTGTATGGCTTTGCGAAAAGCCTGCTGAAAG
CGCTGAAAGAAGATGGCGATGCGGTTATTGTGGTGTTTGATGCGAAAGCGCCG
AGCTTTCGTCATGAAGCGTATGGCGGCTATAAAGCGGGTCGTGCGCCGACCCC
GGAAGATTTTCCGCGTCAGCTGGCCCTGATTAAAGAACTGGTGGATCTGCTGG
GCCTGGCGCGTCTGGAAGTGCCGGGCTATGAAGCGGATGATGTGCTGGCCAGC
CTGGCCAAAAAAGCGGAAAAAGAAGGCTACGAAGTTCGTATTCTGACCGCCG
ATAAAGACCTGTATCAGCTGCTGTCTGATCGTATTCATGTGCTGCATCCTGAGG
GTTATCTGATTACCCCGGCGTGGCTGTGGGAAAAATATGGCCTGCGTCCGGAT
CAGTGGGCGGATTATCGTGCGCTGACCGGCGATGAAAGCGATAACCTGCCGGG
CGTGAAAGGCATTGGCGAAAAAACCGCGCGTAAACTGCTGGAAGAATGGGGC
AGCCTGGAAGCGCTGCTGAAAAACCTGGATCGTCTGAAACCGGCGATTCGTGA
AAAGATCTTAGCGCACATGGATGATCTGAAACTGAGCTGGGATCTGGCCAAAG
TGCGTACCGATCTGCCGCTGGAAGTGGATTTTGCGAAACGTCGTGAACCGGAT
CGTGAACGTCTGCGTGCGTTTCTGGAACGTCTGGAATTTGGCAGCCTGCTGCAT
GAATTTGGCCTGCTGGAAAGCGGTGGCGGCGGTTCTGGCGGTGGTGGCAGCAA
CACACCTTTCACAGTCAAAGTCAAGCCTGCCAACAAGTCGCTTGTAGACCCAA
TCTTATGTAATAGCATTGACGAGATTCCGGTGCGTTACGACGAGCCCGTGTATT
TCGACATCGCAACGGAGGAGGATAAGCCAGTCCTTGTTAGTGTGTATCAGCCG
CATTTTGGGAACAAGGTTTATTGCTTGAATTTGTTGCGTGAGAAACCTGCGCGC
TTCAAAGAGTGGTTTTTGAAATTTTCCGAAATCCGCGGATGGGGATTGGACTTC
GACTTGAAGATTCTGGGCTACACATACGAACAGCTTAAGAACAAAAAAATTGT
AGATGTACAGCTGGCAATCAAAGTTCAACATTATGAACGTTTCAAACAAGGAG
GAACCAAAGGCGAGGGCTTTCGCCTGGACGAGGTTGCACGCGACTTACTTGGT
ATCGAGTACCCCATGGACAAGAGTAAGATCCGTATGACGTTCCGCAACAATAT
GTTCTCTAGTTTCTCTTACGAACAGTTGCTGTACGCGTCTTTGGACGCCTATATC
CCCCACTTATTATATGAACGTTTGAGTTCTTCGACCTTAAACTCGCTTGTTTATC
AAATTGACCAAGAGGTACAGAAGATCGTCGTAGAGACGAGCCAGCATGGTAT
GCCTGTCAAATTACAGGCGTTAGAGGAGGAGATCCACCGTCTGTTACAAATTA
AAAACCAGATTCAAAAAGAGATTCCGTTCAATTATAACAGTCCGCAACAGACG
GCTAAGTTCTTCGGAGTTAACTCCTCTAGCAAAGACGTCTTGATGGACCTGGTA
CTGAAAGGGAATGAGATGGCGAAAAAGGTGTTGGAAGCCCGTCAAGTAGAAA
AGTCCTTAGCCTTCGCTAAGGATTTGTATGATCTGGCGAAGCGCTCGGGCGGA
CGCATTTATGGTAATTTCTTCACTACAACCGCTCCATCGGGGCGTATGTCTTGT
TCCGACATTAACTTACAGAATATTCCACGCCGCTTGCGCCAATTTATTGGGTTT
GAAACTGAAGATAAGAAACTGATTACGGCGGATTTCCCGCAGATCGAGTTACG
TTTAGCTGGGGTGATTTGGAACGAACCGGAATTCATTAACGCGTTCCGTAAGG
GTTTGGACTTGCATAAACTGACAGCTTCAATCCTTTTTGAGAAGAACATCGAG
GAGGTCAGCAAAGAAGAACGCCAAATCGGTAAATCTGCTAATTTCGGCTTGAT
CTACGGGATCTCTCCCCGCGGTTTCGCGGAGTACTGTATTAGTAATGGTATCAA
CATGACCGAGGAAATGGCCGTGGAGATTGTTCGCAAATGGAAAAAATTCTACC
GTAAGATTGCAGAGCAACAGAAGAAGGCGTATGAACGTTTCAAGTACGACGA
ATACGTTGATAATGAGACTTGGTTGAACCGCCCCTATCGTGCATATAAGCCGC
AAGACTTACTTAACTATCAGATTCAGGGCTCGGGAGCCGAGTTGTTTAAGAAG
GCAATTATCCTGATCAAAGAAGTACGTCCGGATTTAAAGCTGGTAAATCTTGT
ACATGACGAAATCGTAGCCGAAGCACTGACCGACGAAGCCGAGGATATTGCA
ATGTTAATTAAACAGAAGATGGAAGAAGCTTGGGATTATTGTCTTGAGAAGGC
CAAAGAATTCGGAAACAAGGTGAGCGAAATTAAATTGGATATTGAGAAGCCT
AACATCTCTCATGTATGGGAAAAAGAA

Codon optimized PS-6739 Taq fusion DNA sequence (with mutations)
Length: 2,631, Type: DNA, Source: Synthetic
SEQ ID NO. 4
ATGCGTGGTATGCTTCCACTGTTTGAACCGAAAGGCCGTGTGCTGCTGGTTGAT
GGCCACCATCTGGCCTATCGTACCTTCCATGCGCTGAAAGGCCTGACGACCAG
CCGCGGCGAACCGGTGCAGGCGGTGTATGGCTTTGCGAAAAGCCTGCTGAAAG
CGCTGAAAGAAGATGGCGATGCGGTTATTGTGGTGTTTGATGCGAAAGCGCCG
AGCTTTCGTCATGAAGCGTATGGCGGCTATAAAGCGGGTCGTGCGCCGACCCC
GGAAGATTTTCCGCGTCAGCTGGCCCTGATTAAAGAACTGGTGGATCTGCTGG
GCCTGGCGCGTCTGGAAGTGCCGGGCTATGAAGCGGATGATGTGCTGGCCAGC
CTGGCCAAAAAAGCGGAAAAAGAAGGCTACGAAGTTCGTATTCTGACCGCCG
ATAAAGACCTGTATCAGCTGCTGTCTGATCGTATTCATGTGCTGCATCCTGAGG
GTTATCTGATTACCCCGGCGTGGCTGTGGGAAAAATATGGCCTGCGTCCGGAT
CAGTGGGCGGATTATCGTGCGCTGACCGGCGATGAAAGCGATAACCTGCCGGG
CGTGAAAGGCATTGGCGAAAAAACCGCGCGTAAACTGCTGGAAGAATGGGGC
AGCCTGGAAGCGCTGCTGAAAAACCTGGATCGTCTGAAACCGGCGATTCGTGA
AAAGATCTTAGCGCACATGGATGATCTGAAACTGAGCTGGGATCTGGCCAAAG
TGCGTACCGATCTGCCGCTGGAAGTGGATTTTGCGAAACGTCGTGAACCGGAT
CGTGAACGTCTGCGTGCGTTTCTGGAACGTCTGGAATTTGGCAGCCTGCTGCAT
GAATTTGGCCTGCTGGAAAGCGGTGGCGGCGGTTCTGGCGGTGGTGGCAGCAA
TATCCAGAAATCAATCCTTAAACCGCAGCCCAAAGCCTTAGTAGAACCCGTTT
TGTGCAACTCCATCGACGAAATTCCAGCAAAGTTTAATGAGCCAATTTATTTCG
ATTTGGCGACTGACGAAGACCGTCCGGTTTTGGCATCGATCTATCAACCGCATT
TTGAGCGCAAGGTGTATTGCCTGAACCTGCTTAAAGAGAAACCGACCCGCTTT
AAAGAGTGGTTGTTAAAGTTTAGTGAAATCCGCGGGTGGGGGTTAGATTTTGA
CCTGCGCGTCTTGGGATACACCTATGAGCAGTTGAAGGACAAAAAGATTGTCG
ATGTCCAATTAGCAATTAAAGTACAGCACTATGAGCGTTTCCGTCAAAATGGG
ACCAAAGGAGAAGGGTTCCGTCTGGATGACGTAGCCCGCGATCTGTTTGGCAT
CGAATATCCAATGGATAAGTCAAAAATCCGTACAACGTTTAAGCAAAACATGT
ACAATACATTCAGCGAGCAGCAGTTACTTTACGCCTCGTTAGACGCATACATTC
CTCATCTGTTATACGAGCAACTTTCCTCATCCACATTAAACAGCTTGGTTTATC
AGTTGGATCAAACGGCACAAAAGATCGTCGTCGAGACCTCTCAGCATGGAATG
CCTGTCAAACTTAAAGCCTTGGAAGAAGAGATCTATCGCTTGACCCAGTTACG
CAACCAAATGCAGAAGGAAATTCCGTTTAACTATAACTCCCCCAAGCAGACCG
CAAAATTTTTCGGCCTGGATAGTAGCAGCAAAGACGTATTGATGGACCTTGCC
CTTCAAGGGAACGAAATGGCTAAGAAAGTCCTTGAGGCACGCCAAATTGAAA
AATCCTTGACATTCGCTAAGGATCTTTACGACTTAGCAAAGAAGAGCGGAGGG
CGCATTTATGGGAACTTCTTTACTACGACTGCCCCTAGCGGACGCATGTCATGT
TCGGATATTAACCTGCAAAACATTCCTCGCCGTCTGCGCCAATTCATCGGGTTT
GACACGGAGGACAAGAAATTAATTACAGCAGACTTCCCGCAAATTGAATTGCG
CTTGGCTGGCGTAATCTGGAACGAGCCCAAATTTATCGAAGCCTTCCGCCAGG
GCATTGACTTGCATAAGCTTACTGCTAGTATTTTATTTGACAAACAATCTATTG
ACGAAGTGTCTAAAGAAGAGCGCCAAATCGGCAAAAGCGCGAATTTCGGCCT
GATTTACGGTATCAGCCCGCGTGGATTTGCCGAGCATTGCATCACTAACGGGA
TCAATATTACTGAAGAGCAGGCGTATGAGATCGTTAAAAAATGGAAGAAGTAC
TATACTAAGATTACCGAGCAACAGAAGAAAGCATATGAACGCTTCAAATATAA
TGAGTATGTCGACAACGAGACATGGCTGAACCGCACATATCGTGCATATAAGC
CACAAGATCTTTTAAACTATCAGATCCAGGGGAGCGGCGCAGAGTTATTCAAA
AAAGCGATTATCCTTTTGAAGCAAGAAGAGCCCTCCCTGAAGATTGTAAACTT
AGTACACGATGAAATTGTCGTGGAAGCTGATTCCAAGGATGCACAGGATCTGG
CGAAACTGATTAAGGAAAAGATGGAAGAAGCGTGGGATTGGTGCTTGGAAAA
GGCGGAGGAATTCGGGAACCGCGTCGCGAAGATCAAGTTAGAAGTCGAGGAA
CCCCACGTTGGGGAGGTCTGGGAGAAAGGC

OS-1622 Taq nuclease domain fusion (with mutations)
Length: 876, Type: Protein, Source: Expression from synthetic
gene OS-1622-Taq-mut
SEQ ID NO. 5
MRGMLPLFEPKGRVLLVDGHHLAYRTFHALKGLTTSRGEPVQAVYGFAKSLLKA
LKEDGDAVIVVFDAKAPSFRHEAYGGYKAGRAPTPEDFPRQLALIKELVDLLGLA
RLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALL
KNLDRLKPAIREKILAHMDDLKLSWDLAKVRTDLPLEVDFAKRREPDRERLRAFL
ERLEFGSLLHEFGLLESGGGGSGGGGSNIPKPILKPQPKALVEPVLCDSVDEIPTKF
NEPIYFDLATDGDRPVLASIYQPHFERKVYCLNLLKEKPTRFKEWLLKFSEIRGWG
LDFDLRALGYTYEQLRDKKIVDVQLAIKVQHHERFKQNGTKGEGFRLDDVARDL
LGIEYPMDKTKIRETFKNNIFHSFSNEQLLYASLDAYIPHLLYEQLTSSTLNSLVYQL
DQQAQKIVVETSQNGMPVKLKALEEEIHRLTQLRNQMQKEIPFNYNSPKQTAKFF
RVDSSSKDVLMDLALQGNEMAKRVLEARQVEKSLAFAKDLYDIAKRSGGRVYG
NFFTTTAPSGRMSCSDINLQNIPRRLRQFIGFDTEDKRLITADFPQIELRLAGVIWNE
SEFIEAFKQGIDLHKLTASILFEKNIEEVGKEERQIGKSANFGLIYGIAPKGFAEYCIT
NGINMTEEQAYEIVRKWKKYYTKIAEQQKKAYERFKYNEYVDNETWLNRTYRA
WKPQDLLNYQIQGSGAELFKKAIVLLKEAKPDLKIVNLVHDEIVVEADSKEAQDL
AKLIKEKMEEAWDWCLEKAEEFGNRVAKIKLEVEQPNVGDTWEKS

OP-2605 Taq nuclease domain fusion (with mutations)
Length: 877, Type: Protein, Source: Expression from synthetic
gene OP-2605-Taq-mut
SEQ ID NO. 6
MRGMLPLFEPKGRVLLVDGHHLAYRTFHALKGLTTSRGEPVQAVYGFAKSLLKA
LKEDGDAVIVVFDAKAPSFRHEAYGGYKAGRAPTPEDFPRQLALIKELVDLLGLA
RLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALL
KNLDRLKPAIREKILAHMDDLKLSWDLAKVRTDLPLEVDFAKRREPDRERLRAFL
ERLEFGSLLHEFGLLESGGGGSGGGGSNTTTLSVKQEVKSLVKPVVCDSIDKIPAK
FDEPVYFDLATDNDKPVLASIYQSHFGHDVYCLNLLKEKPARLKDWLLKFSEIRG
WGLDYDLRVLGYTYEQLKDKKIVDVQLAIKVQHYERFRQNGAKGEGFKLDDVA
RDLLGIEYPMDKTKIRTTFKQNMYNSFNKDQLLYASLDAYIPHLLYEQLSSNTLNS
LVYQLDQQVQKIGIETSQHGLPVRLQALQEEIDKLSQIKKRIQKEIPFNYNSPKQTT
QYLGIDSSSKDVLMDLALKGNELAKKILEARQIEKALTFAKDLYDLAKRNNGRIY
GNFFTTTAPSGRMSCSDINLQNIPRKLRPFIGFETEDKKLITADFPQIELRLAGVIWN
EPKFIEAFNQGIDLHKLTASILFDKRSVDEVSKEERQIGKSANFGLIYGISPKGFAEY
CITNGINMTEEIAYEIVKKWKKYYTKITEQQKKAYERFKYGEYVDNETWLNRTYR
AYKPQDLLNYQIQGSGAELFKKAIILLKEEEPSVKIVNLVHDEIVVEADSKDAQDV
ANLIKEKMGQAWDYCLDKAKEFGNRVAEIKLEVEEPNVSEVWEKG

CS-2729 Taq nuclease domain fusion (with mutations)
Length: 877,Type: Protein, Source: Expression from synthetic
gene CS-2729-Taq-mut
SEQ ID NO. 7
MRGMLPLFEPKGRVLLVDGHHLAYRTFHALKGLTTSRGEPVQAVYGFAKSLLKA
LKEDGDAVIVVFDAKAPSFRHEAYGGYKAGRAPTPEDFPRQLALIKELVDLLGLA
RLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALL
KNLDRLKPAIREKILAHMDDLKLSWDLAKVRTDLPLEVDFAKRREPDRERLRAFL
ERLEFGSLLHEFGLLESGGGGSGGGGSNTPFTVKVKPANKSLVDPILCNSIDEIPVR
YDEPVYFDIATEEDKPVLVSVYQPHFGNKVYCLNLLREKPARFKEWFLKFSEIRG
WGLDFDLKILGYTYEQLKNKKIVDVQLAIKVQHYERFKQGGTKGEGFRLDEVAR
DLLGIEYPMDKSKIRMTFRNNMFSSFSYEQLLYASLDAYIPHLLYERLSSSTLNSLV
YQIDQEVQKIVVETSQHGMPVKLQALEEEIHRLLQIKNQIQKEIPFNYNSPQQTAKF
FGVNSSSKDVLMDLVLKGNEMAKKVLEARQVEKSLAFAKDLYDLAKRSGGRIYG
NFFTTTAPSGRMSCSDINLQNIPRRLRQFIGFETEDKKLITADFPQIELRLAGVIWNE
PEFINAFRKGLDLHKLTASILFEKNIEEVSKEERQIGKSANFGLIYGISPRGFAEYCIS
NGINMTEEMAVEIVRKWKKFYRKIAEQQKKAYERFKYDEYVDNETWLNRPYRA
YKPQDLLNYQIQGSGAELFKKAIILIKEVRPDLKLVNLVHDEIVAEALTDEAEDIAM
LIKQKMEEAWDYCLEKAKEFGNKVSEIKLDIEKPNISHVWEKE

PS-6739 Taq nuclease domain fusion (with mutations)
Length: 877, Type: Protein, Source: Expression from synthetic
gene PS-6739-Taq-mut
SEQ ID NO. 8
MRGMLPLFEPKGRVLLVDGHHLAYRTFHALKGLTTSRGEPVQAVYGFAKSLLKA
LKEDGDAVIVVFDAKAPSFRHEAYGGYKAGRAPTPEDFPRQLALIKELVDLLGLA
RLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALL
KNLDRLKPAIREKILAHMDDLKLSWDLAKVRTDLPLEVDFAKRREPDRERLRAFL
ERLEFGSLLHEFGLLESGGGGSGGGGSNIQKSILKPQPKALVEPVLCNSIDEIPAKFN
EPIYFDLATDEDRPVLASIYQPHFERKVYCLNLLKEKPTRFKEWLLKFSEIRGWGL
DFDLRVLGYTYEQLKDKKIVDVQLAIKVQHYERFRQNGTKGEGFRLDDVARDLF
GIEYPMDKSKIRTTFKQNMYNTFSEQQLLYASLDAYIPHLLYEQLSSSTLNSLVYQ
LDQTAQKIVVETSQHGMPVKLKALEEEIYRLTQLRNQMQKEIPFNYNSPKQTAKFF
GLDSSSKDVLMDLALQGNEMAKKVLEARQIEKSLTFAKDLYDLAKKSGGRIYGN
FFTTTAPSGRMSCSDINLQNIPRRLRQFIGFDTEDKKLITADFPQIELRLAGVIWNEP
KFIEAFRQGIDLHKLTASILFDKQSIDEVSKEERQIGKSANFGLIYGISPRGFAEHCIT
NGINITEEQAYEIVKKWKKYYTKITEQQKKAYERFKYNEYVDNETWLNRTYRAY
KPQDLLNYQIQGSGAELFKKAIILLKQEEPSLKIVNLVHDEIVVEADSKDAQDLAK
LIKEKMEEAWDWCLEKAEEFGNRVAKIKLEVEEPHVGEVWEKG

Codon optimized O15 variant DNA sequence
Length: 2,631, Type: DNA, Source: Synthetic
SEQ ID NO. 9
ATGCGTGGTATGCTTCCACTGTTTGAACCGAAAGGCCGTGTGCTGCTGGT
TGATGGCCACCATCTGGCCTATCGTACCTTCCATGCGCTGAAAGGCCTGA
CGACCAGCCGCGGCGAACCGGTGCAGGCGGTGTATGGCTTTGCGAAAAGC
CTGCTGAAAGCGCTGAAAGAAGATGGCGATGCGGTTATTGTGGTGTTTGA
TGCGAAAGCGCCGAGCTTTCGTCATGAAGCGTATGGCGGCTATAAAGCGG
GTCGTGCGCCGACCCCGGAAGATTTTCCGCGTCAGCTGGCCCTGATTAAA
GAACTGGTGGATCTGCTGGGCCTGGCGCGTCTGGAAGTGCCGGGCTATGA
AGCGGATGATGTGCTGGCCAGCCTGGCCAAAAAAGCGGAAAAAGAAGGCT
ACGAAGTTCGTATTCTGACCGCCGATAAAGACCTGTATCAGCTGCTGTCT
GATCGTATTCATGTGCTGCATCCTGAGGGTTATCTGATTACCCCGGCGTG
GCTGTGGGAAAAATATGGCCTGCGTCCGGATCAGTGGGCGGATTATCGTG
CGCTGACCGGCGATGAAAGCGATAACCTGCCGGGCGTGAAAGGCATTGGC
GAAAAAACCGCGCGTAAACTGCTGGAAGAATGGGGCAGCCTGGAAGCGCT
GCTGAAAAACCTGGATCGTCTGAAACCGGCGATTCGTGAAAAGATCTTAG
CGCACATGGATGATCTGAAACTGAGCTGGGATCTGGCCAAAGTGCGTACC
GATCTGCCGCTGGAAGTGGATTTTGCGAAACGTCGTGAACCGGATCGTGA
ACGTCTGCGTGCGTTTCTGGAACGTCTGGAATTTGGCAGCCTGCTGCATG
AATTTGGCCTGCTGGAAAGCGGTGGCGGCGGTTCTGGCGGTGGTGGCAGC
AATACTACTACATTAAGTGTGAAGCAGGAGGTAAAATCCCTTGTTAAACC
GGTAGTGTGCGATTCGATTGATAAAATTCCAGCAAAGTTCGATGAACCCG
TTTATTTTGATCTTGCTACCGACAATGACAAGCCTGTTTTGGCCTCTATC
TATCAATCTCATTTTGGACATGACGTCTACTGCTTGAACTTATTAAAGGA
GAAACCAGCCCGCCTGAAAGATTGGTTGTTGAAATTCAGCGAGATTCGTG
GCTGGGGTTTAGATTATGACTTGCGCGTTCTTGGCTATACTTATGAACAA
CTTAAAGACAAAAAAATTGTAGACGTACAACTTGCTATTAAGGTGCAACA
CTACGAACGTTTTCGCCAGAACGGAGCGAAGGGCGAGGGTTTCAAGCTTG
ACGATGTCGCCCGCGACCTGTTGGGAATCGAATACCCCATGGACAAGACG
AAAATCCGTACTACCTTCAAGCAAAATATGTATAATTCTTTTAATAAAGA
CCAGTTATTGTATGCCAGCCTGGATGCTTACATCCCTCACTTGCTTTACG
AGCAACTGAGTTCAAATACTTTGAACAGTTTGGTCTATCAGCTGGACCAG
CAAGTTCAAAAGATCGGCATCGAGACGTCACAACATGGTCTTCCTGTCCG
TCTGCAAGCATTGCAAGAAGAGATTGATAAGTTATCACAGATCAAGAAAC
GCATTCAGAAAGAGATCCCATTCAATTATAACTCCCCTAAACAAACCACC
CAGTACTTGGGCATCGATAGCTCCAGTAAGGACGTGTTGATGGACCTGGC
GTTAAAGGGCAACGAGTTAGCTAAGAAAATCCTTGAGGCTCGTCAAATTG
AAAAGGCTCTGACCTTCGCTAAAGAgTTATACGATTTGGCGAAGCGTAAT
AACGGACGTATTTACGGTAACTTCTTTACTACTACCGCGCCATCTGGGCG
TATGTCGTGTAGCGACATCAACTTGCAAAACATTCCACGCAAGTTGCGTC
CGTTCATTGGCTTTGAAACTGAAGATAAGcgtCTGATTACCGCTGATTTT
CCCCAAATCGAATTGCGCTTGGCTGGTGTAATCTGGAACGAAagtAAGTT
TATTGAAGCCTTCAATCAAGGAATTGACTTACACAAGTTGACAGCATCAA
TTCTGTTCGgcAAGCGCTCGGTCGATGAGGTCAGTAAAGAAGAGCGCCAG
ATCGGGAAGTCTGCAAACTTTGGGTTGATCTATGGGATCTCCCCGcgtGG
ATTCGCTGAGTACTGCATCACTAATGGAATCAACATGACCGAAGAGATCG
CATACGAGATCGTCAAGAAGTGGAAAcgtTATTATACAAAAATCACTGAA
CAACAAAAGAAGGCGTATGAACGCTTCAAATACGGGGAGTACGTCGATAA
CGAAACCTGGTTAgccCGTACCTATCGTGCCTATAAACCCCAGGACTTGT
TGAACTACCAGATCCAGGGTTCTGGGGCTGAGCTGTTCAAAAAAGCTATC
ATCCTGTTGAAAGAGGAGGAGCCAAGTGTTAAAATTGTCAACTTGGTCCA
TGATGAAATCGTTGTTGAGGCTGATAGTAAAGATGCTCAGGACGTAGCCA
ATTTAATTAAAGAAAAGATGGGGCAGGCCTGGGATTACTGCTTGGATAAG
GCCAAAGAATTCGGAAACCGCGTAGCGGAAATTAAGCTTGAAGTAGAAGA
GCCCAATGTCAGTGAAGTTTGGGAAAAGGGC

Engineered O15 variant polymerase Length: 877, Type: Protein,
Source: Expression from synthetic gene
SEQ ID NO. 10
MRGMLPLFEPKGRVLLVDGHHLAYRTFHALKGLTTSRGEPVQAVYGFAKSLLKA
LKEDGDAVIVVFDAKAPSFRHEAYGGYKAGRAPTPEDFPRQLALIKELVDLLGLA
RLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALL
KNLDRLKPAIREKILAHMDDLKLSWDLAKVRTDLPLEVDFAKRREPDRERLRAFL
ERLEFGSLLHEFGLLESGGGGSGGGGSNTTTLSVKQEVKSLVKPVVCDSIDKIPAK
FDEPVYFDLATDNDKPVLASIYQSHFGHDVYCLNLLKEKPARLKDWLLKFSEIRG
WGLDYDLRVLGYTYEQLKDKKIVDVQLAIKVQHYERFRQNGAKGEGFKLDDVA
RDLLGIEYPMDKTKIRTTFKQNMYNSFNKDQLLYASLDAYIPHLLYEQLSSNTLNS
LVYQLDQQVQKIGIETSQHGLPVRLQALQEEIDKLSQIKKRIQKEIPFNYNSPKQTT
QYLGIDSSSKDVLMDLALKGNELAKKILEARQIEKALTFAKELYDLAKRNNGRIY
GNFFTTTAPSGRMSCSDINLQNIPRKLRPFIGFETEDKRLITADFPQIELRLAGVIWN
ESKFIEAFNQGIDLHKLTASILFGKRSVDEVSKEERQIGKSANFGLIYGISPRGFAEY
CITNGINMTEEIAYEIVKKWKRYYTKITEQQKKAYERFKYGEYVDNETWLARTYR
AYKPQDLLNYQIQGSGAELFKKAIILLKEEEPSVKIVNLVHDEIVVEADSKDAQDV
ANLIKEKMGQAWDYCLDKAKEFGNRVAEIKLEVEEPNVSEVWEKG

Codon optimized O57 variant DNA sequence
Length: 2,631, Type: DNA, Source: Synthetic
SEQ ID NO. 11
ATGCGTGGTATGCTTCCACTGTTTGAACCGAAAGGCCGTGTGCTGCTGGT
TGATGGCCACCATCTGGCCTATCGTACCTTCCATGCGCTGAAAGGCCTGA
CGACCAGCCGCGGCGAACCGGTGCAGGCGGTGTATGGCTTTGCGAAAAGC
CTGCTGAAAGCGCTGAAAGAAGATGGCGATGCGGTTATTGTGGTGTTTGA
TGCGAAAGCGCCGAGCTTTCGTCATGAAGCGTATGGCGGCTATAAAGCGG
GTCGTGCGCCGACCCCGGAAGATTTTCCGCGTCAGCTGGCCCTGATTAAA
GAACTGGTGGATCTGCTGGGCCTGGCGCGTCTGGAAGTGCCGGGCTATGA
AGCGGATGATGTGCTGGCCAGCCTGGCCAAAAAAGCGGAAAAAGAAGGCT
ACGAAGTTCGTATTCTGACCGCCGATAAAGACCTGTATCAGCTGCTGTCT
GATCGTATTCATGTGCTGCATCCTGAGGGTTATCTGATTACCCCGGCGTG
GCTGTGGGAAAAATATGGCCTGCGTCCGGATCAGTGGGCGGATTATCGTG
CGCTGACCGGCGATGAAAGCGATAACCTGCCGGGCGTGAAAGGCATTGGC
GAAAAAACCGCGCGTAAACTGCTGGAAGAATGGGGCAGCCTGGAAGCGCT
GCTGAAAAACCTGGATCGTCTGAAACCGGCGATTCGTGAAAAGATCTTAG
CGCACATGGATGATCTGAAACTGAGCTGGGATCTGGCCAAAGTGCGTACC
GATCTGCCGCTGGAAGTGGATTTTGCGAAACGTCGTGAACCGGATCGTGA
ACGTCTGCGTGCGTTTCTGGAACGTCTGGAATTTGGCAGCCTGCTGCATG
AATTTGGCCTGCTGGAAAGCGGTGGCGGCGGTTCTGGCGGTGGTGGCAGC
AATACTACTACATTAAGTGTGAAGCAGGAGGTAAAATCCCTTGTTAAACC
GGTAGTGTGCGATTCGATTGATAAAATTCCAGCAAAGTTCGATGAACCCG
TTTATTTTGATCTTGCTACCGACAATGACAAGCCTGTTTTGGCCTCTATC
TATCAATCTCATTTTGGACATGACGTCTACTGCTTGAACTTATTAAAGGA
GAAACCAGCCCGCCTGAAAGATTGGTTGTTGAAATTCAGCGAGATTCGTG
GCTGGGGTTTAGATTATGACTTGCGCGTTCTTGGCTATACTTATGAACAA
CTTAAAGACAAAAAAATTGTAGACGTACAACTTGCTATTAAGGTGCAACA
CTACGAACGTTTTCGCCAGAACGGAGCGAAGGGCGAGGGTTTCAAGCTTG
ACGATGTCGCCCGCGACCTGTTGGGAATCGAATACCCCATGGACAAGACG
AAAATCCGTACTACCTTCAAGCAAAATATGTATAATTCTTTTAATAAAGA
CCAGTTATTGTATGCCAGCCTGGATGCTTACATCCCTCACTTGCTTTACG
AGCAACTGAGTTCAAATACTTTGAACAGTTTGGTCTATCAGCTGGACCAG
CAAGTTCAAAAGATCGGCATCGAGACGTCACAACATGGTCTTCCTGTCCG
TCTGCAAGCATTGCAAGAAGAGATTGATAAGTTATCACAGATCAAGAAAC
GCATTCAGAAAGAGATCCCATTCAATTATAACTCCCCTAAACAAACCACC
CAGTACTTGGGCATCGATAGCTCCAGTAAGGACGTGTTGATGGACCTGGC
GTTAAAGGGCAACGAGTTAGCTAAGAAAATCCTTGAGGCTCGTCAAATTG
AAAAGGCTCTGACCTTCGCTAAAGtgTTATACGATTTGGCGAAGCGTAAT
AACGGACGTATTTACGGTAACTTCTTTACTACTACCGCGCCATCTGGGCG
TATGTCGTGTAGCGACATCAACTTGCAAAACATTCCACGCAAGTTGCGTC
CGTTCATTGGCTTTGAAACTGAAGATAAGcgtCTGATTACCGCTGATTTT
CCCCAAATCGAATTGCGCTTGGCTGGTGTAATCTGGAACGAAaagAAGTT
TATTGAAGCCTTCAATCAAGGAATTGACTTACACAAGTTGACAGCATCAA
TTCTGTTCGgcAAGCGCTCGGTCGATGAGGTCAGTAAAGAAGAGCGCCAG
ATCGGGAAGTCTGCAAACTTTGGGTTGATCTATGGGATCTCCCCGcgtGG
ATTCGCTGAGTACTGCATCACTAATGGAATCAACATGACCGAAGAGATCG
CATACGAGATCGTCAAGAAGTGGAAAgcgTATTATACAAAAATCACTGAA
CAACAAAAGAAGGCGTATGAACGCTTCAAATACGGGGAGTACGTCGATAA
CGAAACCTGGTTAgccCGTACCTATCGTGCCTATAAACCCCAGGACTTGT
TGAACTACCAGATCCAGGGTTCTGGGGCTGAGCTGTTCAAAAAAGCTATC
ATCCTGTTGAAAGAGGAGGAGCCAAGTGTTAAAATTGTCAACTTGGTCCA
TGATGAAATCGTTGTTGAGGCTGATAGTAAAGATGCTCAGGACGTAGCCA
ATTTAATTAAAGAAAAGATGGGGCAGGCCTGGGATTACTGCTTGGATAAG
GCCAAAGAATTCGGAAACCGCGTAGCGGAAATTAAGCTTGAAGTAGAAGA
GCCCAATGTCAGTGAAGTTTGGGAAAAGGGC

Engineered O57 variant polymerase Length: 877,
Type: Protein, Source: Expression from synthetic gene
SEQ ID NO. 12
MRGMLPLFEPKGRVLLVDGHHLAYRTFHALKGLTTSRGEPVQAVYGFAKSLLKA
LKEDGDAVIVVFDAKAPSFRHEAYGGYKAGRAPTPEDFPRQLALIKELVDLLGLA
RLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALL
KNLDRLKPAIREKILAHMDDLKLSWDLAKVRTDLPLEVDFAKRREPDRERLRAFL
ERLEFGSLLHEFGLLESGGGGSGGGGSNTTTLSVKQEVKSLVKPVVCDSIDKIPAK
FDEPVYFDLATDNDKPVLASIYQSHFGHDVYCLNLLKEKPARLKDWLLKFSEIRG
WGLDYDLRVLGYTYEQLKDKKIVDVQLAIKVQHYERFRQNGAKGEGFKLDDVA
RDLLGIEYPMDKTKIRTTFKQNMYNSFNKDQLLYASLDAYIPHLLYEQLSSNTLNS
LVYQLDQQVQKIGIETSQHGLPVRLQALQEEIDKLSQIKKRIQKEIPFNYNSPKQTT
QYLGIDSSSKDVLMDLALKGNELAKKILEARQIEKALTFAKVLYDLAKRNNGRIY
GNFFTTTAPSGRMSCSDINLQNIPRKLRPFIGFETEDKRLITADFPQIELRLAGVIWN
EKKFIEAFNQGIDLHKLTASILFGKRSVDEVSKEERQIGKSANFGLIYGISPRGFAEY
CITNGINMTEEIAYEIVKKWKAYYTKITEQQKKAYERFKYGEYVDNETWLARTYR
AYKPQDLLNYQIQGSGAELFKKAIILLKEEEPSVKIVNLVHDEIVVEADSKDAQDV
ANLIKEKMGQAWDYCLDKAKEFGNRVAEIKLEVEEPNVSEVWEKG

Codon optimized O58 variant DNA sequence
Length: 2,631, Type: DNA, Source: Synthetic
SEQ ID NO. 13
ATGCGTGGTATGCTTCCACTGTTTGAACCGAAAGGCCGTGTGCTGCTGGT
TGATGGCCACCATCTGGCCTATCGTACCTTCCATGCGCTGAAAGGCCTGA
CGACCAGCCGCGGCGAACCGGTGCAGGCGGTGTATGGCTTTGCGAAAAGC
CTGCTGAAAGCGCTGAAAGAAGATGGCGATGCGGTTATTGTGGTGTTTGA
TGCGAAAGCGCCGAGCTTTCGTCATGAAGCGTATGGCGGCTATAAAGCGG
GTCGTGCGCCGACCCCGGAAGATTTTCCGCGTCAGCTGGCCCTGATTAAA
GAACTGGTGGATCTGCTGGGCCTGGCGCGTCTGGAAGTGCCGGGCTATGA
AGCGGATGATGTGCTGGCCAGCCTGGCCAAAAAAGCGGAAAAAGAAGGCT
ACGAAGTTCGTATTCTGACCGCCGATAAAGACCTGTATCAGCTGCTGTCT
GATCGTATTCATGTGCTGCATCCTGAGGGTTATCTGATTACCCCGGCGTG
GCTGTGGGAAAAATATGGCCTGCGTCCGGATCAGTGGGCGGATTATCGTG
CGCTGACCGGCGATGAAAGCGATAACCTGCCGGGCGTGAAAGGCATTGGC
GAAAAAACCGCGCGTAAACTGCTGGAAGAATGGGGCAGCCTGGAAGCGCT
GCTGAAAAACCTGGATCGTCTGAAACCGGCGATTCGTGAAAAGATCTTAG
CGCACATGGATGATCTGAAACTGAGCTGGGATCTGGCCAAAGTGCGTACC
GATCTGCCGCTGGAAGTGGATTTTGCGAAACGTCGTGAACCGGATCGTGA
ACGTCTGCGTGCGTTTCTGGAACGTCTGGAATTTGGCAGCCTGCTGCATG
AATTTGGCCTGCTGGAAAGCGGTGGCGGCGGTTCTGGCGGTGGTGGCAGC
AATACTACTACATTAAGTGTGAAGCAGGAGGTAAAATCCCTTGTTAAACC
GGTAGTGTGCGATTCGATTGATAAAATTCCAGCAAAGTTCGATGAACCCG
TTTATTTTGATCTTGCTACCGACAATGACAAGCCTGTTTTGGCCTCTATC
TATCAATCTCATTTTGGACATGACGTCTACTGCTTGAACTTATTAAAGGA
GAAACCAGCCCGCCTGAAAGATTGGTTGTTGAAATTCAGCGAGATTCGTG
GCTGGGGTTTAGATTATGACTTGCGCGTTCTTGGCTATACTTATGAACAA
CTTAAAGACAAAAAAATTGTAGACGTACAACTTGCTATTAAGGTGCAACA
CTACGAACGTTTTCGCCAGAACGGAGCGAAGGGCGAGGGTTTCAAGCTTG
ACGATGTCGCCCGCGACCTGTTGGGAATCGAATACCCCATGGACAAGACG
AAAATCCGTACTACCTTCAAGCAAAATATGTATAATTCTTTTAATAAAGA
CCAGTTATTGTATGCCAGCCTGGATGCTTACATCCCTCACTTGCTTTACG
AGCAACTGAGTTCAAATACTTTGAACAGTTTGGTCTATCAGCTGGACCAG
CAAGTTCAAAAGATCGGCATCGAGACGTCACAACATGGTCTTCCTGTCCG
TCTGCAAGCATTGCAAGAAGAGATTGATAAGTTATCACAGATCAAGAAAC
GCATTCAGAAAGAGATCCCATTCAATTATAACTCCCCTAAACAAACCACC
CAGTACTTGGGCATCGATAGCTCCAGTAAGGACGTGTTGATGGACCTGGC
GTTAAAGGGCAACGAGTTAGCTAAGAAAATCCTTGAGGCTCGTCAAATTG
AAAAGGCTCTGACCTTCGCTAAAGagTTATACGATTTGGCGAAGCGTAAT
AACGGACGTATTTACGGTAACTTCTTTACTACTACCGCGCCATCTGGGCG
TATGTCGTGTAGCGACATCAACTTGCAAAACATTCCACGCAAGTTGCGTC
CGTTCATTGGCTTTGAAACTGAAGATAAGcgtCTGATTACCGCTGATTTT
CCCCAAATCGAATTGCGCTTGGCTGGTGTAATCTGGAACGAAaagAAGTT
TATTGAAGCCTTCAATCAAGGAATTGACTTACACAAGTTGACAGCATCAA
TTCTGTTCGaaAAGCGCTCGGTCGATGAGGTCAGTAAAGAAGAGCGCCAG
ATCGGGAAGTCTGCAAACTTTGGGTTGATCTATGGGATCTCCCCGcgtGG
ATTCGCTGAGTACTGCATCACTAATGGAATCAACATGACCGAAGAGATCG
CATACGAGATCGTCAAGAAGTGGAAAcgtTATTATACAAAAATCACTGAA
CAACAAAAGAAGGCGTATGAACGCTTCAAATACGGGGAGTACGTCGATAA
CGAAACCTGGTTAgccCGTACCTATCGTGCCTATAAACCCCAGGACTTGT
TGAACTACCAGATCCAGGGTTCTGGGGCTGAGCTGTTCAAAAAAGCTATC
ATCCTGTTGAAAGAGGAGGAGCCAAGTGTTAAAATTGTCAACTTGGTCCA
TGATGAAATCGTTGTTGAGGCTGATAGTAAAGATGCTCAGGACGTAGCCA
ATTTAATTAAAGAAAAGATGGGGCAGGCCTGGGATTACTGCTTGGATAAG
GCCAAAGAATTCGGAAACCGCGTAGCGGAAATTAAGCTTGAAGTAGAAGA
GCCCAATGTCAGTGAAGTTTGGGAAAAGGGC

Engineered O58 variant polymerase
Length: 877, Type: Protein, Source: Expression from synthetic gene
SEQ ID NO. 14
MRGMLPLFEPKGRVLLVDGHHLAYRTFHALKGLTTSRGEPVQAVYGFAKSLLKA
LKEDGDAVIVVFDAKAPSFRHEAYGGYKAGRAPTPEDFPRQLALIKELVDLLGLA
RLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALL
KNLDRLKPAIREKILAHMDDLKLSWDLAKVRTDLPLEVDFAKRREPDRERLRAFL
ERLEFGSLLHEFGLLESGGGGSGGGGSNTTTLSVKQEVKSLVKPVVCDSIDKIPAK
FDEPVYFDLATDNDKPVLASIYQSHFGHDVYCLNLLKEKPARLKDWLLKFSEIRG
WGLDYDLRVLGYTYEQLKDKKIVDVQLAIKVQHYERFRQNGAKGEGFKLDDVA
RDLLGIEYPMDKTKIRTTFKQNMYNSFNKDQLLYASLDAYIPHLLYEQLSSNTLNS
LVYQLDQQVQKIGIETSQHGLPVRLQALQEEIDKLSQIKKRIQKEIPFNYNSPKQTT
QYLGIDSSSKDVLMDLALKGNELAKKILEARQIEKALTFAKELYDLAKRNNGRIY
GNFFTTTAPSGRMSCSDINLQNIPRKLRPFIGFETEDKRLITADFPQIELRLAGVIWN
EKKFIEAFNQGIDLHKLTASILFEKRSVDEVSKEERQIGKSANFGLIYGISPRGFAEY
CITNGINMTEEIAYEIVKKWKRYYTKITEQQKKAYERFKYGEYVDNETWLARTYR
AYKPQDLLNYQIQGSGAELFKKAIILLKEEEPSVKIVNLVHDEIVVEADSKDAQDV
ANLIKEKMGQAWDYCLDKAKEFGNRVAEIKLEVEEPNVSEVWEKG

OS-1622 Taq nuclease domain fusion (without mutation)
Length: 876, Type: Protein, Source: Expression from synthetic gene
SEQ ID NO. 15
MRGMLPLFEPKGRVLLVDGHHLAYRTFHALKGLTTSRGEPVQAVYGFAKSLLKA
LKEDGDAVIVVFDAKAPSFRHEAYGGYKAGRAPTPEDFPRQLALIKELVDLLGLA
RLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALL
KNLDRLKPAIREKILAHMDDLKLSWDLAKVRTDLPLEVDFAKRREPDRERLRAFL
ERLEFGSLLHEFGLLESGGGGSGGGGSNIPKPILKPQPKALVEPVLCDSVDEIPTKF
NEPIYFDLATDGDRPVLASIYQPHFERKVYCLNLLKEKPTRFKEWLLKFSEIRGWG
LDFDLRALGYTYEQLRDKKIVDVQLAIKVQHHERFKQNGTKGEGFRLDDVARDL
LGIEYPMDKTKIRETFKNNIFHSFSNEQLLYASLDAYIPHLLYEQLTSSTLNSLVYQL
DQQAQKIVVETSQNGMPVKLKALEEEIHRLTQLRNQMQKEIPFNYNSPKQTAKFF
RVDSSSKDVLMDLALQGNEMAKRVLEARQVEKSLAFAKDLYDIAKRSGGRVYG
NFFTTTAPSGRMSCSDINLQqIPRRLRQFIGFDTEDKRLITADFPQIELRLAGVIWNE
SEFIEAFKQGIDLHKLTASILFEKNIEEVGKEERQIGKSANFGLIYGIAPKGFAEYCIT
NGINMTEEQAYEIVRKWKKYYTKIAEQhqvAYERFKYNEYVDNETWLNRTYRAW
KPQDLLNYQIQGSGAELFKKAIVLLKEAKPDLKIVNLVHDEIVVEADSKEAQDLAK
LIKEKMEEAWDWCLEKAEEFGNRVAKIKLEVEQPNVGDTWEKS

OP-2605 Taq nuclease domain fusion (without mutation)
Length: 877, Type: Protein, Source: Expression from synthetic gene
OP-2605-Taq-wt
SEQ ID NO. 16
MRGMLPLFEPKGRVLLVDGHHLAYRTFHALKGLTTSRGEPVQAVYGFAKSLLKA
LKEDGDAVIVVFDAKAPSFRHEAYGGYKAGRAPTPEDFPRQLALIKELVDLLGLA
RLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALL
KNLDRLKPAIREKILAHMDDLKLSWDLAKVRTDLPLEVDFAKRREPDRERLRAFL
ERLEFGSLLHEFGLLESGGGGSGGGGSNTTTLSVKQEVKSLVKPVVCDSIDKIPAK
FDEPVYFDLATDNDKPVLASIYQSHFGHDVYCLNLLKEKPARLKDWLLKFSEIRG
WGLDYDLRVLGYTYEQLKDKKIVDVQLAIKVQHYERFRQNGAKGEGFKLDDVA
RDLLGIEYPMDKTKIRTTFKQNMYNSFNKDQLLYASLDAYIPHLLYEQLSSNTLNS
LVYQLDQQVQKIGIETSQHGLPVRLQALQEEIDKLSQIKKRIQKEIPFNYNSPKQTT
QYLGIDSSSKDVLMDLALKGNELAKKILEARQIEKALTFAKDLYDLAKRNNGRIY
GNFFTTTAPSGRMSCSDINLQqIPRKLRPFIGFETEDKKLITADFPQIELRLAGVIWN
EPKFIEAFNQGIDLHKLTASILFDKRSVDEVSKEERQIGKSANFGLIYGISPKGFAEY
CITNGINMTEEIAYEIVKKWKKYYTKITEQhqvAYERFKYGEYVDNETWLNRTYRA
YKPQDLLNYQIQGSGAELFKKAIILLKEEEPSVKIVNLVHDEIVVEADSKDAQDVA
NLIKEKMGQAWDYCLDKAKEFGNRVAEIKLEVEEPNVSEVWEKG

CS-2729 Taq nuclease domain fusion (without mutation)
Length: 877, Type: Protein, Source: Expression from synthetic gene
SEQ ID NO. 17
MRGMLPLFEPKGRVLLVDGHHLAYRTFHALKGLTTSRGEPVQAVYGFAKSLLKA
LKEDGDAVIVVFDAKAPSFRHEAYGGYKAGRAPTPEDFPRQLALIKELVDLLGLA
RLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALL
KNLDRLKPAIREKILAHMDDLKLSWDLAKVRTDLPLEVDFAKRREPDRERLRAFL
ERLEFGSLLHEFGLLESGGGGSGGGGSNTPFTVKVKPANKSLVDPILCNSIDEIPVR
YDEPVYFDIATEEDKPVLVSVYQPHFGNKVYCLNLLREKPARFKEWFLKFSEIRG
WGLDFDLKILGYTYEQLKNKKIVDVQLAIKVQHYERFKQGGTKGEGFRLDEVAR
DLLGIEYPMDKSKIRMTFRNNMFSSFSYEQLLYASLDAYIPHLLYERLSSSTLNSLV
YQIDQEVQKIVVETSQHGMPVKLQALEEEIHRLLQIKNQIQKEIPFNYNSPQQTAKF
FGVNSSSKDVLMDLVLKGNEMAKKVLEARQVEKSLAFAKDLYDLAKRSGGRIYG
NFFTTTAPSGRMSCSDINLQqIPRRLRQFIGFETEDKKLITADFPQIELRLAGVIWNEP
EFINAFRKGLDLHKLTASILFEKNIEEVSKEERQIGKSANFGLIYGISPRGFAEYCISN
GINMTEEMAVEIVRKWKKFYRKIAEQhqlAYERFKYDEYVDNETWLNRPYRAYKP
QDLLNYQIQGSGAELFKKAIILIKEVRPDLKLVNLVHDEIVAEALTDEAEDIAMLIK
QKMEEAWDYCLEKAKEFGNKVSEIKLDIEKPNISHVWEKE

CS-2729-Taq-wt

PS-6739 Taq nuclease domain fusion (without mutation)
Length: 877, Type: Protein, Source: Expression from synthetic gene
PS-6739-Taq-wt
SEQ ID NO. 18
MRGMLPLFEPKGRVLLVDGHHLAYRTFHALKGLTTSRGEPVQAVYGFAKSLLKA
LKEDGDAVIVVFDAKAPSFRHEAYGGYKAGRAPTPEDFPRQLALIKELVDLLGLA
RLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALL
KNLDRLKPAIREKILAHMDDLKLSWDLAKVRTDLPLEVDFAKRREPDRERLRAFL
ERLEFGSLLHEFGLLESGGGGSGGGGSNIQKSILKPQPKALVEPVLCNSIDEIPAKFN
EPIYFDLATDEDRPVLASIYQPHFERKVYCLNLLKEKPTRFKEWLLKFSEIRGWGL
DFDLRVLGYTYEQLKDKKIVDVQLAIKVQHYERFRQNGTKGEGFRLDDVARDLF
GIEYPMDKSKIRTTFKQNMYNTFSEQQLLYASLDAYIPHLLYEQLSSSTLNSLVYQ
LDQTAQKIVVETSQHGMPVKLKALEEEIYRLTQLRNQMQKEIPFNYNSPKQTAKFF
GLDSSSKDVLMDLALQGNEMAKKVLEARQIEKSLTFAKDLYDLAKKSGGRIYGN
FFTTTAPSGRMSCSDINLQqIPRRLRQFIGFDTEDKKLITADFPQIELRLAGVIWNEP
KFIEAFRQGIDLHKLTASILFDKQSIDEVSKEERQIGKSANFGLIYGISPRGFAEHCIT
NGINITEEQAYEIVKKWKKYYTKITEQhqiAYERFKYNEYVDNETWLNRTYRAYKP
QDLLNYQIQGSGAELFKKAIILLKQEEPSLKIVNLVHDEIVVEADSKDAQDLAKLIK
EKMEEAWDWCLEKAEEFGNRVAKIKLEVEEPHVGEVWEKG

Linker
SEQ ID NO. 19
GGGGSGGGGS

Putative viral gene product. Locus tag JGI20132J14458_100001622
Length: 1607, Type: Protein, Source: Synthetic
SEQ ID NO. 20
MRSISFFELLVKIGLIVEDEYGYTFPDYVLVLTQTPEGIELKEIKDAFLRWNETNKE
KWVEEFEEYCKLARERNRYYLSLFAEKRNAQDFFKRTKVAIRIDIDEPLKLDQILEI
VNNRELLPIQPTHILRTIKGWHIFYITQDFIECDDKEILYMIHSYVEDLKSNLRKHAD
KIDHTYSIATRYSNEIYELREPYTKKELLEEMNKYYDTDILINGLPVKRREYSRIPIS
QISEGLALTLWNACPVIRSLEEKWETHTYNEWFILSWKYAFLYVLTQKEEYKQEF
LQKSKFWKGKVVIAPEQQFRNTLKWMLKDRETLPYFSCSFVHRRVVDADEKCKN
CQYARWIFDEYGERKLISNWFKDLFYLETRLEGFKVDEKRNLWVKEDTNEPVCEL
FKIEDVVLYNKPNRKEKYIKIFYRDKYEFIPYVLTASANTDFSEFIVLTFYNQQLFK
KLLTNYLTLFQLARGVREIDKAGYKYNDLKRKWDMVVANMDSFRAEDLNFYM
WSDRTNRLNYYIPIVNGSFEAWKNAYRRVVKAKDPIMLILLGHFISHITKEYFRDK
FVASSEPNVLIFLRGFTTTGKTTRLRIASALYGTPQVIQITETTTAKILREFGNIGMPL
PLDEFRMRKDKEEEIANMIYAIANEASKDTAYERFSPIQVPVVFSGEKNALAVEVL
CKNREGLYRRSIVLDVDELPKQKNTALVEFYTNEILPILKYNHGYIFKLIDFIENHV
DIEALAQYYKDVEILRNEFDKKRSKVLRGIVKSLDNHLKLIYASIHVFLEFLGLSDE
EKANVFVILEQYIRNVFAKFYDTLLPKEESKLNKIIDYLRDLADGLYNASNNPIKKT
TIRGLTIKKLIDIAGVQVPTTDIEPYLKLLFMKYYENKKAFVYLGSIFVEGRNPAWF
EGMVTREYERLTYIKEHHPEFYKSILEVFTELMLSIHGEAGLRRLHNLFVESFKFED
LKDFIDNNGGDNTPPDEDLPSGDDDDNTPPNDNLPPVEEFDYENKENEDNEEEDEL
EKHFTGEDGLSLPKRMNIPKPILKPQPKALVEPVLCDSVDEIPTKFNEPIYFDLETDG
DRPVLASIYQPHFERKVYCLNLLKEKPTRFKEWLLKFSEIRGWGLDFDLRALGYTY
EQLRDKKIVDVQLAIKVQHHERFKQNGTKGEGFRLDDVARDLLGIEYPMDKTKIR
ETFKNNIFHSFSNEQLLYASLDAYIPHLLYEQLTSSTLNSLVYQLDQQAQKIVVETS
QNGMPVKLKALEEEIHRLTQLRNQMQKEIPFNYNSPKQTAKFFRVDSSSKDVLMD
LALQGNEMAKRVLEARQVEKSLAFAKDLYDIAKRSGGRVYGNFFTTTAPSGRMS
CSDINLQQIPRRLRQFIGFDTEDKRLITADFPQIELRLAGVIWNESEFIEAFKQGIDLH
KLTASILFEKNIEEVGKEERQIGKSANFGLIYGIAPKGFAEYCITNGINMTEEQAYEI
VRKWKKYYTKIAEQHQVAYERFKYNEYVDNETWLNRTYRAWKPQDLLNYQIQG
SGAELFKKAIVLLKEAKPDLKIVNLVHDEIVVEADSKEAQDLAKLIKEKMEEAWD
WCLEKAEEFGNRVAKIKLEVEQPNVGDTWEKS

Putative viral gene product. Locus tag Ga0186926_122605
Length: 1595, Type: Protein, Source: Synthetic
SEQ ID NO. 21
MNKITFFDLFVKIGLVYENEKYGYTFNDYVLVLAETLEGVAVKEIRDAFLGFNEA
DKERWKKEFEEYCKVARERNRYFLSLFAEKRNSFDYFKRTKVAIRIDIDEPLKLEE
VLELVNNRDLIPIPPTHILRSVKGWHIFYITQDYIESVDREVLYFIHSYTEELKSLLRK
HADKVDHTYQIATRFSEEIYELREPYTKEKLFQAINDYYGVEIQINGLTVKRGQYG
KIPVAHLSEGVALTLWNACPVLRQLEERWENHTYDEWFLMSWKYAFLYALTQKE
EYKQEFLQKSKLWKGQVKTTPEQQFQYTLKWILKDRETLPYFSCSFVHKSVEGAE
EKCNSCQYARWMLDENGERRLISNWFKDLFYLETRLEGFKIDERKNVWVKEDTE
EPVCELFKIEDVVLYNKPNNKQKYIKIFYRDKYEFIPYVLTASANTDFSEFIVLTFY
NQQLFKKLLTNYLTLFQLARGVREIDKAGYKYNDLKKRWDTVVANVGAFRVED
LNFYMWNDRTSRLNYYIPVVNGSFEAWKDAYRRVVKAKDPILLILLGHFISHITKE
YFKDKFVASSEPNVLIFLRGFTTAGKTTRLRIASALYGTPQAIQITETTTAKILREFG
NIGTPLPLDEFRMRKDKEEEVANMIYAIANESAKDTAYERFNPIQVPVVFSGEKNA
LSVETLCKNRDGLYRRSIVLDIDEIPKQKNSSLVEFYTNKILPILKYHHGYIFKFIDFI
ENEVDIETVAERFKDVELLNEELNKKKSKVFRGIVKSLDNHLKMIIASLSVFLDFLN
LNEEEKADIYIALDHYIRNVLAKFYDTLLPKEEDKLSKIIDYLRDFADGLYNASNNP
IKKTTIKGLTTKKLIDVAGMQVPTTDIEPYLRLLFMKYYQSNRGYTYLGSIFVEGR
NPAWFESMIKIEYERLIHIKEQHPTYYKNALEVFVELMLSIHGELGLRRLYRIFVKT
YKFDDLKDFISDNNDDTPPDDNPPNGDDGDDDLPPDDSISPNGHYTEDPEEPHFEE
ETNSFSQNTTTLSVKQEVKSLVKPVVCDSIDKIPAKFDEPVYFDLETDNDKPVLASI
YQSHFGHDVYCLNLLKEKPARLKDWLLKFSEIRGWGLDYDLRVLGYTYEQLKDK
KIVDVQLAIKVQHYERFRQNGAKGEGFKLDDVARDLLGIEYPMDKTKIRTTFKQN
MYNSFNKDQLLYASLDAYIPHLLYEQLSSNTLNSLVYQLDQQVQKIGIETSQHGLP
VRLQALQEEIDKLSQIKKRIQKEIPFNYNSPKQTTQYLGIDSSSKDVLMDLALKGNE
LAKKILEARQIEKALTFAKDLYDLAKRNNGRIYGNFFTTTAPSGRMSCSDINLQQIP
RKLRPFIGFETEDKKLITADFPQIELRLAGVIWNEPKFIEAFNQGIDLHKLTASILFDK
RSVDEVSKEERQIGKSANFGLIYGISPKGFAEYCITNGINMTEEIAYEIVKKWKKYY
TKITEQHQVAYERFKYGEYVDNETWLNRTYRAYKPQDLLNYQIQGSGAELFKKAI
ILLKEEEPSVKIVNLVHDEIVVEADSKDAQDVANLIKEKMGQAWDYCLDKAKEFG
NRVAEIKLEVEEPNVSEVWEKG

Putative viral gene product. Locus tag Ga0080008_15802729
Length: 1619, Type: Protein, Source: Synthetic
SEQ ID NO. 22
MNRITFFDLFVKCGLIYDDEEYGYRFTPYVLVLAETVDGIGIKPITDLFFGFNETDR
ERWVKEFLSYCKEARERNRYYLSVFSERRNSFDFFKRTKAAIRIDIDEPLTLSEVIK
LVENKDLIPIQPTHVLRSVRGWHILYITKDFIENDEQNKNIFYLLHSYAEDLKSNLR
KYADKVDYTYQIATRFSEEIYELREPYEVKELIKAIEDYYSLDIEINGFKLKRRQFG
RIPISHISEGVALTLWNACPVLRRLEEKWEYHTYNEWFIMSWKYAFLYALTGKSE
YKEEFLNKSKLWKGVVKMTPEQQFEYTLKWVLKEKETLPYFSCSFVYKHVSEAE
EKCKECPYARWQEDEFGNKTLISSWFKELFYIESRLENFKIDEKRNLWVKADTNEP
ICELFKIEDVVLYNKPNKKERFIKIFYRNKYEFVPYVLTASANMDFSEFNVLTFYNQ
TLFKNLLINYLNLFQLSRGAREIDKAGYKYNRITKSWDKVVANLGNFRVEDLNFF
MWNDRTNELRYYIPVVNGSYEVWRETYKKVLLAKDPIMLIILGHFLSHITREYFKD
KFVSSNEPNVLIFLRGFTTSGKTTRLKIASALYGTPEVIQITETTTAKILREFGNIGMP
LPLDEFRMRKDKEEEVANMIYAIANEAAKDTAYERFNPISVPVVFSGEKNTLFVET
LAKNREGLYRRSIVLDVDEIPKPEREQLAEFYAREIYPVLRKNHGFIYKFIEFLENEA
DIDRLSELYQDVELLREEFDKRRSKVLRGIVRSLDNHLKMILASLHLFVDFIGLNDE
EKAEVYMCVEDYIKTKLVGFYETFLPKEEDKLTRIIDYLRDIIDGLYNAWKHPVNK
KTIKRLTINKLIEIAGVQAPTQDLEPYLKLLLMKYYPSNNTFTYVGSVFVEGRNYLS
DDYAKLETERLLFVKGRYPHLYQDILEVFVELMLIVHGEYGLSKLIKYMKKLGFT
DVMEYTIKHNITIHKFGDDEDDNPSPTSPPKNPPEISPQNNSSSTEITSTSEVDEDLV
NSFVGEEGFSSATLKTDTTKQQNQTNTPFTVKVKPANKSLVDPILCNSIDEIPVRYD
EPVYFDIETEEDKPVLVSVYQPHFGNKVYCLNLLREKPARFKEWFLKFSEIRGWGL
DFDLKILGYTYEQLKNKKIVDVQLAIKVQHYERFKQGGTKGEGFRLDEVARDLLG
IEYPMDKSKIRMTFRNNMFSSFSYEQLLYASLDAYIPHLLYERLSSSTLNSLVYQID
QEVQKIVVETSQHGMPVKLQALEEEIHRLLQIKNQIQKEIPFNYNSPQQTAKFFGV
NSSSKDVLMDLVLKGNEMAKKVLEARQVEKSLAFAKDLYDLAKRSGGRIYGNFF
TTTAPSGRMSCSDINLQQIPRRLRQFIGFETEDKKLITADFPQIELRLAGVIWNEPEFI
NAFRKGLDLHKLTASILFEKNIEEVSKEERQIGKSANFGLIYGISPRGFAEYCISNGI
NMTEEMAVEIVRKWKKFYRKIAEQHQLAYERFKYDEYVDNETWLNRPYRAYKP
QDLLNYQIQGSGAELFKKAIILIKEVRPDLKLVNLVHDEIVAEALTDEAEDIAMLIK
QKMEEAWDYCLEKAKEFGNKVSEIKLDIEKPNISHVWEKE

Putative viral gene product. Locus tag Ga0079997_11796739
Length: 1608, Type: Protein, Source: Synthetic
SEQ ID NO. 23
MKSISFSELFVKIGLVSETDDGYTFNDYVLVLSQTPEGTVLKEIREAFLGFNETDKE
RWVKEFEEYCKEARERNRYYLSLFAEKRNSQDYLKRTKVAIRIDIDEPLKLEQVLE
IVNNGDLIPIPPTHLLRTIKGWHIFYITKDFIENEDKEVIYLIHSYTEELKTHLRKYAD
KIDHTYQIATRYSTEIYELREPYTKEELLKAINDYFGVEIQVNGLIVKRKDCSGVPV
SQLSEGLALTLWNACPVLRSLEERWETHTYHEWFILSWKHAFLYVLTQKEEYRQE
FLQKSKLWKGKVVITPEQQFQNTLKWMLKDRETLPYFSCSFVYKYVADAGEKCE
KCQYARWVFDENGERKLISNWFRDLFYLETRLEGFRVDEKRNLWVKEDTGEPVC
ELFKIEDVVLYNKPNRKEKYIKIFYRDKYEFIPYVLTASANTDFSEFIVLTFYNQQLF
KYLLNKYLTLFQLARGVREIDKAGYKYNDLKRKWDMVVANMGSFRAEDLNFYM
WNDRTNRLNYYIPIMNGSFETWKNTYRRVVKAKDPIMLLLLGHFISHITKEYFRDK
FVASSEPNVLIFLRGFTTAGKTTRLRIASALYGTPQVIQITETTTAKILREFGNIGMPL
PLDEFKMRKDKEEEVANMIYAIANEASKDTAYERFNPIQVPVVFSGEKNALSVEK
LCANREGLYRRSIVLDVDELPKQKNSALIDFYTSELLPILKYNHGYIFKLIDFIENNL
DIEALTQLYKDVEILKDEFDKRKSKALRGIVKSLDNHLKLIFASIHVFLEFLDLSEEE
KAEVFAILEEYIRNVLAKFYDTLLPKEENKLSKIVDYLRDLADGLYNASNNPIKKT
TIRGLTLKKLIDVAGVQVPTTDIEPYVKMLFMRYYESKKGYVYLGSIFVEGRNPA
WFEGMVAREYERLIYIKQHYPELYRSILEVFAELMLSIHGEAGLRRVHSIFVESFKF
DDLKDFLNNNNDDNTPPDDLPPNGGDDDDTPPDDLPPTEEFDYENEEDEEDEEEE
DELNEHFAGEDGLTTPKMMNIQKSILKPQPKALVEPVLCNSIDEIPAKFNEPIYFDL
ETDEDRPVLASIYQPHFERKVYCLNLLKEKPTRFKEWLLKFSEIRGWGLDFDLRVL
GYTYEQLKDKKIVDVQLAIKVQHYERFRQNGTKGEGFRLDDVARDLFGIEYPMD
KSKIRTTFKQNMYNTFSEQQLLYASLDAYIPHLLYEQLSSSTLNSLVYQLDQTAQK
IVVETSQHGMPVKLKALEEEIYRLTQLRNQMQKEIPFNYNSPKQTAKFFGLDSSSK
DVLMDLALQGNEMAKKVLEARQIEKSLTFAKDLYDLAKKSGGRIYGNFFTTTAPS
GRMSCSDINLQQIPRRLRQFIGFDTEDKKLITADFPQIELRLAGVIWNEPKFIEAFRQ
GIDLHKLTASILFDKQSIDEVSKEERQIGKSANFGLIYGISPRGFAEHCITNGINITEE
QAYEIVKKWKKYYTKITEQHQIAYERFKYNEYVDNETWLNRTYRAYKPQDLLNY
QIQGSGAELFKKAIILLKQEEPSLKIVNLVHDEIVVEADSKDAQDLAKLIKEKMEEA
WDWCLEKAEEFGNRVAKIKLEVEEPHVGEVWEKG

Core family A polymerase OS-1622
Length: 576, Type: Protein, Source: Synthetic
SEQ ID NO. 24
NIPKPILKPQPKALVEPVLCDSVDEIPTKFNEPIYFDLETDGDRPVLASIYQPHFERK
VYCLNLLKEKPTRFKEWLLKFSEIRGWGLDFDLRALGYTYEQLRDKKIVDVQLAI
KVQHHERFKQNGTKGEGFRLDDVARDLLGIEYPMDKTKIRETFKNNIFHSFSNEQL
LYASLDAYIPHLLYEQLTSSTLNSLVYQLDQQAQKIVVETSQNGMPVKLKALEEEI
HRLTQLRNQMQKEIPFNYNSPKQTAKFFRVDSSSKDVLMDLALQGNEMAKRVLE
ARQVEKSLAFAKDLYDIAKRSGGRVYGNFFTTTAPSGRMSCSDINLQQIPRRLRQF
IGFDTEDKRLITADFPQIELRLAGVIWNESEFIEAFKQGIDLHKLTASILFEKNIEEVG
KEERQIGKSANFGLIYGIAPKGFAEYCITNGINMTEEQAYEIVRKWKKYYTKIAEQ
HQVAYERFKYNEYVDNETWLNRTYRAWKPQDLLNYQIQGSGAELFKKAIVLLKE
AKPDLKIVNLVHDEIVVEADSKEAQDLAKLIKEKMEEAWDWCLEKAEEFGNRVA
KIKLEVEQPNVGDTWEKS

Core family A polymerase OP-2605
Length: 577, Type: Protein, Source: Synthetic
SEQ ID NO. 25
NTTTLSVKQEVKSLVKPVVCDSIDKIPAKFDEPVYFDLETDNDKPVLASIYQSHFG
HDVYCLNLLKEKPARLKDWLLKFSEIRGWGLDYDLRVLGYTYEQLKDKKIVDVQ
LAIKVQHYERFRQNGAKGEGFKLDDVARDLLGIEYPMDKTKIRTTFKQNMYNSFN
KDQLLYASLDAYIPHLLYEQLSSNTLNSLVYQLDQQVQKIGIETSQHGLPVRLQAL
QEEIDKLSQIKKRIQKEIPFNYNSPKQTTQYLGIDSSSKDVLMDLALKGNELAKKIL
EARQIEKALTFAKDLYDLAKRNNGRIYGNFFTTTAPSGRMSCSDINLQQIPRKLRPF
IGFETEDKKLITADFPQIELRLAGVIWNEPKFIEAFNQGIDLHKLTASILFDKRSVDE
VSKEERQIGKSANFGLIYGISPKGFAEYCITNGINMTEEIAYEIVKKWKKYYTKITE
QHQVAYERFKYGEYVDNETWLNRTYRAYKPQDLLNYQIQGSGAELFKKAIILLKE
EEPSVKIVNLVHDEIVVEADSKDAQDVANLIKEKMGQAWDYCLDKAKEFGNRVA
EIKLEVEEPNVSEVWEKG

Core family A polymerase CS-2729
Length: 577, Type: Protein, Source: Synthetic
SEQ ID NO. 26
NTPFTVKVKPANKSLVDPILCNSIDEIPVRYDEPVYFDIETEEDKPVLVSVYQPHFG
NKVYCLNLLREKPARFKEWFLKFSEIRGWGLDFDLKILGYTYEQLKNKKIVDVQL
AIKVQHYERFKQGGTKGEGFRLDEVARDLLGIEYPMDKSKIRMTFRNNMFSSFSY
EQLLYASLDAYIPHLLYERLSSSTLNSLVYQIDQEVQKIVVETSQHGMPVKLQALE
EEIHRLLQIKNQIQKEIPFNYNSPQQTAKFFGVNSSSKDVLMDLVLKGNEMAKKVL
EARQVEKSLAFAKDLYDLAKRSGGRIYGNFFTTTAPSGRMSCSDINLQQIPRRLRQ
FIGFETEDKKLITADFPQIELRLAGVIWNEPEFINAFRKGLDLHKLTASILFEKNIEEV
SKEERQIGKSANFGLIYGISPRGFAEYCISNGINMTEEMAVEIVRKWKKFYRKIAEQ
HQLAYERFKYDEYVDNETWLNRPYRAYKPQDLLNYQIQGSGAELFKKAIILIKEV
RPDLKLVNLVHDEIVAEALTDEAEDIAMLIKQKMEEAWDYCLEKAKEFGNKVSEI
KLDIEKPNISHVWEKE

Core family A polymerase PS-6739
Length: 577, Type: Protein, Source: Synthetic
SEQ ID NO. 27
NIQKSILKPQPKALVEPVLCNSIDEIPAKFNEPIYFDLETDEDRPVLASIYQPHFERK
VYCLNLLKEKPTRFKEWLLKFSEIRGWGLDFDLRVLGYTYEQLKDKKIVDVQLAI
KVQHYERFRQNGTKGEGFRLDDVARDLFGIEYPMDKSKIRTTFKQNMYNTFSEQQ
LLYASLDAYIPHLLYEQLSSSTLNSLVYQLDQTAQKIVVETSQHGMPVKLKALEEE
IYRLTQLRNQMQKEIPFNYNSPKQTAKFFGLDSSSKDVLMDLALQGNEMAKKVLE
ARQIEKSLTFAKDLYDLAKKSGGRIYGNFFTTTAPSGRMSCSDINLQQIPRRLRQFI
GFDTEDKKLITADFPQIELRLAGVIWNEPKFIEAFRQGIDLHKLTASILFDKQSIDEV
SKEERQIGKSANFGLIYGISPRGFAEHCITNGINITEEQAYEIVKKWKKYYTKITEQH
QIAYERFKYNEYVDNETWLNRTYRAYKPQDLLNYQIQGSGAELFKKAIILLKQEEP
SLKIVNLVHDEIVVEADSKDAQDLAKLIKEKMEEAWDWCLEKAEEFGNRVAKIKL
EVEEPHVGEVWEKG

1. Polymerase selected from the group of,
a. a polymerase (O15) as encoded by a nucleic acid according to SEQ ID NO. 9 or a nucleic acid that is at least 98% identical thereto,
b. a polymerase (O15) with the amino acid sequence according to SEQ ID NO: 10 or a polymerase that is at least 90% identical thereto,
c. a polymerase (O57) as encoded by a nucleic acid according to SEQ ID NO. 11 or a nucleic acid that is at least 98% identical thereto,
d. a polymerase (O57) with the amino acid sequence according to SEQ ID NO: 12 or a polymerase that is at least 90% identical thereto,
e. a polymerase (O58) as encoded by a nucleic acid according to SEQ ID NO. 13 or a nucleic acid that is at least 98% identical thereto, and
f. a polymerase (O58) with the amino acid sequence according to SEQ ID NO: 14 or a polymerase that is at least 90% identical thereto.

2. Polymerase comprising,
a. an N-terminal 5′-3′nuclease domain,
i. stemming from Taq polymerase or,
ii. a polymerase sharing at least 95% amino acid sequence identity with the N-terminal 5′-3′ nuclease domain of Taq polymerase,
b. an adjacent and linked polymerase domain, stemming from a viral family A polymerase, wherein the polymerase domain stems preferably from,
1. JGI20132J14458_100001622 (1607 amino acids; SEQ ID NO. 20), or a functional fragment that shares at least 98% amino acid sequence identity thereto, and is altered to comprise the following amino acid changes, Q627N, H751Q, Q752K, and V753K, or
2. Ga0186926_122605 (1595 amino acids; SEQ ID NO. 21), or a functional fragment that shares at least 98% amino acid sequence identity thereto, and is altered to comprise the following amino acid changes, Q627N, H752Q, Q753K, and V754K, or
3. Ga0080008_15802729 (1619 amino acids; SEQ ID NO. 22) or a functional fragment that shares at least 98% amino acid sequence identity thereto, and is altered to comprise the following amino acid changes, Q628N, H752Q, Q753K, and L754K, or
4. Ga0079997_11796739 (1608 amino acids; SEQ ID NO. 23), or a functional fragment that shares at least 98% amino acid sequence identity thereto and is altered to comprise the following amino acid changes, Q627N, H752Q, Q753K, and I754K.

3. Polymerase according to claim 2, wherein
a. there is a peptide linker between the exonuclease domain and the polymerase domain and,
b. optionally said peptide linker has the amino acid sequence according to SEQ ID NO. 19. (GGGGSGGGGS).

4. Polymerase according to claim 2 or 3, wherein polymerase domain is codon optimized for expression in E. coli.

5. Polymerase comprising,
a. the amino acid sequence of
i. SEQ ID NO. 16 (OP-2605) comprising the following additional amino acid changes, Q627N, H752Q, Q753K, and V754K,
ii. or an amino acid sequence at least 95%, preferably at least 98% identical thereto,
b. the amino acid sequence of
i. SEQ ID NO. 15 (OS-1622) comprising the following additional amino acid changes, Q627N, H751Q, Q752K, and V753K,
ii. or an amino acid sequence at least 90%, preferably at least 95%, more preferably at least 98% identical thereto,
c. the amino acid sequence of
i. SEQ ID NO. 17 (CS-2729) comprising the following additional amino acid changes, Q628N, H752Q, Q753K, and L754K, or an amino acid sequence at least 90%, preferably at least 95%, more preferably at least 98% identical thereto, or
d. the amino acid sequence of
i. SEQ ID NO. 18 (PS-6739) comprising the following additional amino acid changes, Q627N, H752Q, Q753K, and I754K,
ii. or an amino acid sequence at least 90%, preferably at least 95%, more preferably at least 98% identical thereto.

6. A method for amplifying template nucleic acids comprising contacting the template nucleic acids with a polymerase according to any one of claims 1 to 5, preferably wherein the method is reverse transcription (RT) PCR.

7. The method according to claim 6, wherein the method comprises:
a) generating cDNA using a polypeptide according to any one of claims 1 to 6, and
b) amplifying the generated cDNA using a polypeptide according to any one of claims 1 to 6.

8. Kit comprising a polymerase according to claims 1 to 5.

9. A vector encoding a polymerase according to any one of claims 1 to 5.

10. A transformed host cell comprising the vector according to claim 9.

11. A viral family A polymerase, or a portion thereof comprising one of the following mutations, selected from the group of
a. Q627N or Q628N;
b. H752Q or H751Q;
c. Q753K or Q752K;
d. V754K or V753K or L754K or I754K;
or mutations in similar residues from locally aligned family A polymerases per the amino acid numbering of polymerases according to claims 1 to 5.

12. Polymerase domain selected from the group of:
(a) OP-2605 (577 amino acids) according to SEQ ID NO. 25 (derived from Locus tag Ga0186926_122605),
(b) OS-1622 (576 amino acids) according to SEQ ID NO. 24 (derived from Locus tag JGI20132J14458_100001622),
(c) CS-2729 (577 amino acids) according to SEQ ID NO. 26 (derived from Locus tag Ga0080008_15802729), or
(d) PS-6739 (577 amino acids) according to SEQ ID NO. 27 (derived from Locus tag Ga0079997_11796739), or
(e) polypeptide polymerase domain or functional fragment that shares more than 80%, 85%, 90%, 95% or 99% sequence identity with (a), (b), (c) or (d).

13. Use of a polymerase domain according to claim 12 for constructing a chimeric enzyme, preferably an enzyme with polymerase activity, more preferably an enzyme with reverse transcriptase activity.


SEQUENCE LISTING (27 sequences)

SEQ ID NO: 1 - DNA, 2628 bp, Artificial Sequence
  synthetic construct; Codon optimized OS-1622 Taq fusion DNA sequence (with mutations)
atgcgtggtatgcttccactgtttgaaccgaaaggccgtgtgctgctggttgatggccaccatctggcctatcgtaccttccatgcgctgaaaggcctgacgaccagccgcggcgaaccggtgcaggcggtgtatggctttgcgaaaagcctgctgaaagcgctgaaagaagatggcgatgcggttattgtggtgtttgatgcgaaagcgccgagctttcgtcatgaagcgtatggcggctataaagcgggtcgtgcgccgaccccggaagattttccgcgtcagctggccctgattaaagaactggtggatctgctgggcctggcgcgtctggaagtgccgggctatgaagcggatgatgtgctggccagcctggccaaaaaagcggaaaaagaaggctacgaagttcgtattctgaccgccgataaagacctgtatcagctgctgtctgatcgtattcatgtgctgcatcctgagggttatctgattaccccggcgtggctgtgggaaaaatatggcctgcgtccggatcagtgggcggattatcgtgcgctgaccggcgatgaaagcgataacctgccgggcgtgaaaggcattggcgaaaaaaccgcgcgtaaactgctggaagaatggggcagcctggaagcgctgctgaaaaacctggatcgtctgaaaccggcgattcgtgaaaagatcttagcgcacatggatgatctgaaactgagctgggatctggccaaagtgcgtaccgatctgccgctggaagtggattttgcgaaacgtcgtgaaccggatcgtgaacgtctgcgtgcgtttctggaacgtctggaatttggcagcctgctgcatgaatttggcctgctggaaagcggtggcggcggttctggcggtggtggcagcaacattcccaagccgatccttaaaccacaacctaaagcacttgttgaaccggttctgtgcgacagcgtcgatgaaatccccacaaagtttaacgaaccaatctatttcgatcttgcaaccgacggggaccgcccggtgttagcatccatctaccaaccccactttgaacgtaaggtctattgtcttaacttattaaaagagaagcctactcgttttaaggagtggcttctgaagttcagcgagattcgtggctggggtttagacttcgatctgcgcgccttaggttacacatacgagcagttacgcgataaaaagattgtggacgtgcagctggctatcaaagtccagcatcatgaacgcttcaagcagaacggtactaagggtgaaggctttcgtctggacgacgtggcccgcgatttgttaggaatcgagtaccctatggataagaccaagatccgcgagacgtttaaaaataacatttttcactcatttagcaatgagcaattgttgtatgcatctcttgacgcttatatccctcacctgctttacgaacaattaacgagttcaacgcttaattcgctggtttaccagttagatcagcaagcacagaaaattgtggtggaaacaagtcagaatggtatgccggttaaattaaaggctctggaagaggaaatccatcgcttgacgcagcttcgtaaccaaatgcaaaaagaaattccttttaactacaattcgcctaaacagacagctaaattcttccgtgttgattccagcagtaaggacgttcttatggacctggcattacaaggtaatgagatggcgaaacgcgttttggaagcccgccaggtcgagaagagcctggccttcgctaaggatctttatgacatcgcgaaacgcagcggagggcgcgtttatggaaatttctttaccacaacggcgccgagtggacgtatgagttgtagcgatatcaaccttcaaaatatccctcgccgcttacgccaattcattggctttgatacggaagataaacgtcttattacggcagactttcctcaaatcgagctgcgcttagcgggagtcatctggaacgagagcgagttcattgaagcctttaaacaaggcattgaccttcataaattaacggcgtcaattctgtttgagaagaatattgaggaagtcgggaaggaggaacgtcagattggtaaatcggcgaattttggattaatttatggaattgctcctaaaggttttgctgagtactgtattacgaacggaattaatatgacggaagagcaggcatacgaaattgtacgcaagtggaagaaatattatactaagattgcggagcagcaaaaaaaggcttatgaacgtttcaaatataacgagtacgtggacaacgaaacatggctgaatcgcacctaccgtgcatggaaaccacaagatttgttaaactaccagatccaaggatctggtgctgagttgttcaagaaggccattgtcctgctgaaggaggcaaaaccggatcttaagatcgtcaacttggtacacgatgagattgttgtcgaggccgactctaaggaagcccaagaccttgccaagctgatcaaagagaagatggaagaagcctgggactggtgtttggaaaaggcggaggagttcggcaaccgtgtagccaagattaaacttgaagtagagcagccgaacgtaggggatacatgggagaaatcg

SEQ ID NO: 2 - DNA, 2631 bp, Artificial Sequence
  synthetic construct; Codon optimized OP-2605 Taq fusion DNA sequence (with mutations)
atgcgtggtatgcttccactgtttgaaccgaaaggccgtgtgctgctggttgatggccaccatctggcctatcgtaccttccatgcgctgaaaggcctgacgaccagccgcggcgaaccggtgcaggcggtgtatggctttgcgaaaagcctgctgaaagcgctgaaagaagatggcgatgcggttattgtggtgtttgatgcgaaagcgccgagctttcgtcatgaagcgtatggcggctataaagcgggtcgtgcgccgaccccggaagattttccgcgtcagctggccctgattaaagaactggtggatctgctgggcctggcgcgtctggaagtgccgggctatgaagcggatgatgtgctggccagcctggccaaaaaagcggaaaaagaaggctacgaagttcgtattctgaccgccgataaagacctgtatcagctgctgtctgatcgtattcatgtgctgcatcctgagggttatctgattaccccggcgtggctgtgggaaaaatatggcctgcgtccggatcagtgggcggattatcgtgcgctgaccggcgatgaaagcgataacctgccgggcgtgaaaggcattggcgaaaaaaccgcgcgtaaactgctggaagaatggggcagcctggaagcgctgctgaaaaacctggatcgtctgaaaccggcgattcgtgaaaagatcttagcgcacatggatgatctgaaactgagctgggatctggccaaagtgcgtaccgatctgccgctggaagtggattttgcgaaacgtcgtgaaccggatcgtgaacgtctgcgtgcgtttctggaacgtctggaatttggcagcctgctgcatgaatttggcctgctggaaagcggtggcggcggttctggcggtggtggcagcaatactactacattaagtgtgaagcaggaggtaaaatcccttgttaaaccggtagtgtgcgattcgattgataaaattccagcaaagttcgatgaacccgtttattttgatcttgctaccgacaatgacaagcctgttttggcctctatctatcaatctcattttggacatgacgtctactgcttgaacttattaaaggagaaaccagcccgcctgaaagattggttgttgaaattcagcgagattcgtggctggggtttagattatgacttgcgcgttcttggctatacttatgaacaacttaaagacaaaaaaattgtagacgtacaacttgctattaaggtgcaacactacgaacgttttcgccagaacggagcgaagggcgagggtttcaagcttgacgatgtcgcccgcgacctgttgggaatcgaataccccatggacaagacgaaaatccgtactaccttcaagcaaaatatgtataattcttttaataaagaccagttattgtatgccagcctggatgcttacatccctcacttgctttacgagcaactgagttcaaatactttgaacagtttggtctatcagctggaccagcaagttcaaaagatcggcatcgagacgtcacaacatggtcttcctgtccgtctgcaagcattgcaagaagagattgataagttatcacagatcaagaaacgcattcagaaagagatcccattcaattataactcccctaaacaaaccacccagtacttgggcatcgatagctccagtaaggacgtgttgatggacctggcgttaaagggcaacgagttagctaagaaaatccttgaggctcgtcaaattgaaaaggctctgaccttcgctaaagatttatacgatttggcgaagcgtaataacggacgtatttacggtaacttctttactactaccgcgccatctgggcgtatgtcgtgtagcgacatcaacttgcaaaacattccacgcaagttgcgtccgttcattggctttgaaactgaagataagaaactgattaccgctgattttccccaaatcgaattgcgcttggctggtgtaatctggaacgaaccaaagtttattgaagccttcaatcaaggaattgacttacacaagttgacagcatcaattctgttcgataagcgctcggtcgatgaggtcagtaaagaagagcgccagatcgggaagtctgcaaactttgggttgatctatgggatctccccgaaaggattcgctgagtactgcatcactaatggaatcaacatgaccgaagagatcgcatacgagatcgtcaagaagtggaaaaaatattatacaaaaatcactgaacaacaaaagaaggcgtatgaacgcttcaaatacggggagtacgtcgataacgaaacctggttaaatcgtacctatcgtgcctataaaccccaggacttgttgaactaccagatccagggttctggggctgagctgttcaaaaaagctatcatcctgttgaaagaggaggagccaagtgttaaaattgtcaacttggtccatgatgaaatcgttgttgaggctgatagtaaagatgctcaggacgtagccaatttaattaaagaaaagatggggcaggcctgggattactgcttggataaggccaaagaattcggaaaccgcgtagcggaaattaagcttgaagtagaagagcccaatgtcagtgaagtttgggaaaagggc

SEQ ID NO: 3 - DNA, 2631 bp, Artificial Sequence
  synthetic construct; Codon optimized CS-2729 Taq fusion DNA sequence (with mutations)
atgcgtggtatgcttccactgtttgaaccgaaaggccgtgtgctgctggttgatggccaccatctggcctatcgtaccttccatgcgctgaaaggcctgacgaccagccgcggcgaaccggtgcaggcggtgtatggctttgcgaaaagcctgctgaaagcgctgaaagaagatggcgatgcggttattgtggtgtttgatgcgaaagcgccgagctttcgtcatgaagcgtatggcggctataaagcgggtcgtgcgccgaccccggaagattttccgcgtcagctggccctgattaaagaactggtggatctgctgggcctggcgcgtctggaagtgccgggctatgaagcggatgatgtgctggccagcctggccaaaaaagcggaaaaagaaggctacgaagttcgtattctgaccgccgataaagacctgtatcagctgctgtctgatcgtattcatgtgctgcatcctgagggttatctgattaccccggcgtggctgtgggaaaaatatggcctgcgtccggatcagtgggcggattatcgtgcgctgaccggcgatgaaagcgataacctgccgggcgtgaaaggcattggcgaaaaaaccgcgcgtaaactgctggaagaatggggcagcctggaagcgctgctgaaaaacctggatcgtctgaaaccggcgattcgtgaaaagatcttagcgcacatggatgatctgaaactgagctgggatctggccaaagtgcgtaccgatctgccgctggaagtggattttgcgaaacgtcgtgaaccggatcgtgaacgtctgcgtgcgtttctggaacgtctggaatttggcagcctgctgcatgaatttggcctgctggaaagcggtggcggcggttctggcggtggtggcagcaacacacctttcacagtcaaagtcaagcctgccaacaagtcgcttgtagacccaatcttatgtaatagcattgacgagattccggtgcgttacgacgagcccgtgtatttcgacatcgcaacggaggaggataagccagtccttgttagtgtgtatcagccgcattttgggaacaaggtttattgcttgaatttgttgcgtgagaaacctgcgcgcttcaaagagtggtttttgaaattttccgaaatccgcggatggggattggacttcgacttgaagattctgggctacacatacgaacagcttaagaacaaaaaaattgtagatgtacagctggcaatcaaagttcaacattatgaacgtttcaaacaaggaggaaccaaaggcgagggctttcgcctggacgaggttgcacgcgacttacttggtatcgagtaccccatggacaagagtaagatccgtatgacgttccgcaacaatatgttctctagtttctcttacgaacagttgctgtacgcgtctttggacgcctatatcccccacttattatatgaacgtttgagttcttcgaccttaaactcgcttgtttatcaaattgaccaagaggtacagaagatcgtcgtagagacgagccagcatggtatgcctgtcaaattacaggcgttagaggaggagatccaccgtctgttacaaattaaaaaccagattcaaaaagagattccgttcaattataacagtccgcaacagacggctaagttcttcggagttaactcctctagcaaagacgtcttgatggacctggtactgaaagggaatgagatggcgaaaaaggtgttggaagcccgtcaagtagaaaagtccttagccttcgctaaggatttgtatgatctggcgaagcgctcgggcggacgcatttatggtaatttcttcactacaaccgctccatcggggcgtatgtcttgttccgacattaacttacagaatattccacgccgcttgcgccaatttattgggtttgaaactgaagataagaaactgattacggcggatttcccgcagatcgagttacgtttagctggggtgatttggaacgaaccggaattcattaacgcgttccgtaagggtttggacttgcataaactgacagcttcaatcctttttgagaagaacatcgaggaggtcagcaaagaagaacgccaaatcggtaaatctgctaatttcggcttgatctacgggatctctccccgcggtttcgcggagtactgtattagtaatggtatcaacatgaccgaggaaatggccgtggagattgttcgcaaatggaaaaaattctaccgtaagattgcagagcaacagaagaaggcgtatgaacgtttcaagtacgacgaatacgttgataatgagacttggttgaaccgcccctatcgtgcatataagccgcaagacttacttaactatcagattcagggctcgggagccgagttgtttaagaaggcaattatcctgatcaaagaagtacgtccggatttaaagctggtaaatcttgtacatgacgaaatcgtagccgaagcactgaccgacgaagccgaggatattgcaatgttaattaaacagaagatggaagaagcttgggattattgtcttgagaaggccaaagaattcggaaacaaggtgagcgaaattaaattggatattgagaagcctaacatctctcatgtatgggaaaaagaa

SEQ ID NO: 4 - DNA, 2631 bp, Artificial Sequence
  synthetic construct; Codon optimized PS-6739 Taq fusion DNA sequence (with mutations)
atgcgtggtatgcttccactgtttgaaccgaaaggccgtgtgctgctggttgatggccaccatctggcctatcgtaccttccatgcgctgaaaggcctgacgaccagccgcggcgaaccggtgcaggcggtgtatggctttgcgaaaagcctgctgaaagcgctgaaagaagatggcgatgcggttattgtggtgtttgatgcgaaagcgccgagctttcgtcatgaagcgtatggcggctataaagcgggtcgtgcgccgaccccggaagattttccgcgtcagctggccctgattaaagaactggtggatctgctgggcctggcgcgtctggaagtgccgggctatgaagcggatgatgtgctggccagcctggccaaaaaagcggaaaaagaaggctacgaagttcgtattctgaccgccgataaagacctgtatcagctgctgtctgatcgtattcatgtgctgcatcctgagggttatctgattaccccggcgtggctgtgggaaaaatatggcctgcgtccggatcagtgggcggattatcgtgcgctgaccggcgatgaaagcgataacctgccgggcgtgaaaggcattggcgaaaaaaccgcgcgtaaactgctggaagaatggggcagcctggaagcgctgctgaaaaacctggatcgtctgaaaccggcgattcgtgaaaagatcttagcgcacatggatgatctgaaactgagctgggatctggccaaagtgcgtaccgatctgccgctggaagtggattttgcgaaacgtcgtgaaccggatcgtgaacgtctgcgtgcgtttctggaacgtctggaatttggcagcctgctgcatgaatttggcctgctggaaagcggtggcggcggttctggcggtggtggcagcaatatccagaaatcaatccttaaaccgcagcccaaagccttagtagaacccgttttgtgcaactccatcgacgaaattccagcaaagtttaatgagccaatttatttcgatttggcgactgacgaagaccgtccggttttggcatcgatctatcaaccgcattttgagcgcaaggtgtattgcctgaacctgcttaaagagaaaccgacccgctttaaagagtggttgttaaagtttagtgaaatccgcgggtgggggttagattttgacctgcgcgtcttgggatacacctatgagcagttgaaggacaaaaagattgtcgatgtccaattagcaattaaagtacagcactatgagcgtttccgtcaaaatgggaccaaaggagaagggttccgtctggatgacgtagcccgcgatctgtttggcatcgaatatccaatggataagtcaaaaatccgtacaacgtttaagcaaaacatgtacaatacattcagcgagcagcagttactttacgcctcgttagacgcatacattcctcatctgttatacgagcaactttcctcatccacattaaacagcttggtttatcagttggatcaaacggcacaaaagatcgtcgtcgagacctctcagcatggaatgcctgtcaaacttaaagccttggaagaagagatctatcgcttgacccagttacgcaaccaaatgcagaaggaaattccgtttaactataactcccccaagcagaccgcaaaatttttcggcctggatagtagcagcaaagacgtattgatggaccttgcccttcaagggaacgaaatggctaagaaagtccttgaggcacgccaaattgaaaaatccttgacattcgctaaggatctttacgacttagcaaagaagagcggagggcgcatttatgggaacttctttactacgactgcccctagcggacgcatgtcatgttcggatattaacctgcaaaacattcctcgccgtctgcgccaattcatcgggtttgacacggaggacaagaaattaattacagcagacttcccgcaaattgaattgcgcttggctggcgtaatctggaacgagcccaaatttatcgaagccttccgccagggcattgacttgcataagcttactgctagtattttatttgacaaacaatctattgacgaagtgtctaaagaagagcgccaaatcggcaaaagcgcgaatttcggcctgatttacggtatcagcccgcgtggatttgccgagcattgcatcactaacgggatcaatattactgaagagcaggcgtatgagatcgttaaaaaatggaagaagtactatactaagattaccgagcaacagaagaaagcatatgaacgcttcaaatataatgagtatgtcgacaacgagacatggctgaaccgcacatatcgtgcatataagccacaagatcttttaaactatcagatccaggggagcggcgcagagttattcaaaaaagcgattatccttttgaagcaagaagagccctccctgaagattgtaaacttagtacacgatgaaattgtcgtggaagctgattccaaggatgcacaggatctggcgaaactgattaaggaaaagatggaagaagcgtgggattggtgcttggaaaaggcggaggaattcgggaaccgcgtcgcgaagatcaagttagaagtcgaggaaccccacgttggggaggtctgggagaaaggc

SEQ ID NO: 5 - protein, 876 aa, Artificial Sequence
  synthetic construct; OS-1622-Taq-mut - OS-1622 Taq nuclease domain fusion (with mutations)
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESGGGGSGGGGSNIPKPILKPQPKALVEPVLCDSVDEIPTKFNEPIYFDLATDGDRPVLASIYQPHFERKVYCLNLLKEKPTRFKEWLLKFSEIRGWGLDFDLRALGYTYEQLRDKKIVDVQLAIKVQHHERFKQNGTKGEGFRLDDVARDLLGIEYPMDKTKIRETFKNNIFHSFSNEQLLYASLDAYIPHLLYEQLTSSTLNSLVYQLDQQAQKIVVETSQNGMPVKLKALEEEIHRLTQLRNQMQKEIPFNYNSPKQTAKFFRVDSSSKDVLMDLALQGNEMAKRVLEARQVEKSLAFAKDLYDIAKRSGGRVYGNFFTTTAPSGRMSCSDINLQNIPRRLRQFIGFDTEDKRLITADFPQIELRLAGVIWNESEFIEAFKQGIDLHKLTASILFEKNIEEVGKEERQIGKSANFGLIYGIAPKGFAEYCITNGINMTEEQAYEIVRKWKKYYTKIAEQQKKAYERFKYNEYVDNETWLNRTYRAWKPQDLLNYQIQGSGAELFKKAIVLLKEAKPDLKIVNLVHDEIVVEADSKEAQDLAKLIKEKMEEAWDWCLEKAEEFGNRVAKIKLEVEQPNVGDTWEKS

SEQ ID NO: 6 - protein, 877 aa, Artificial Sequence
  synthetic construct; OP-2605-Taq-mut - OP-2605 Taq nuclease domain fusion (with mutations)
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESGGGGSGGGGSNTTTLSVKQEVKSLVKPVVCDSIDKIPAKFDEPVYFDLATDNDKPVLASIYQSHFGHDVYCLNLLKEKPARLKDWLLKFSEIRGWGLDYDLRVLGYTYEQLKDKKIVDVQLAIKVQHYERFRQNGAKGEGFKLDDVARDLLGIEYPMDKTKIRTTFKQNMYNSFNKDQLLYASLDAYIPHLLYEQLSSNTLNSLVYQLDQQVQKIGIETSQHGLPVRLQALQEEIDKLSQIKKRIQKEIPFNYNSPKQTTQYLGIDSSSKDVLMDLALKGNELAKKILEARQIEKALTFAKDLYDLAKRNNGRIYGNFFTTTAPSGRMSCSDINLQNIPRKLRPFIGFETEDKKLITADFPQIELRLAGVIWNEPKFIEAFNQGIDLHKLTASILFDKRSVDEVSKEERQIGKSANFGLIYGISPKGFAEYCITNGINMTEEIAYEIVKKWKKYYTKITEQQKKAYERFKYGEYVDNETWLNRTYRAYKPQDLLNYQIQGSGAELFKKAIILLKEEEPSVKIVNLVHDEIVVEADSKDAQDVANLIKEKMGQAWDYCLDKAKEFGNRVAEIKLEVEEPNVSEVWEKG

SEQ ID NO: 7 - protein, 877 aa, Artificial Sequence
  synthetic construct; CS-2729-Taq-mut - CS-2729 Taq nuclease domain fusion (with mutations)
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESGGGGSGGGGSNTPFTVKVKPANKSLVDPILCNSIDEIPVRYDEPVYFDIATEEDKPVLVSVYQPHFGNKVYCLNLLREKPARFKEWFLKFSEIRGWGLDFDLKILGYTYEQLKNKKIVDVQLAIKVQHYERFKQGGTKGEGFRLDEVARDLLGIEYPMDKSKIRMTFRNNMFSSFSYEQLLYASLDAYIPHLLYERLSSSTLNSLVYQIDQEVQKIVVETSQHGMPVKLQALEEEIHRLLQIKNQIQKEIPFNYNSPQQTAKFFGVNSSSKDVLMDLVLKGNEMAKKVLEARQVEKSLAFAKDLYDLAKRSGGRIYGNFFTTTAPSGRMSCSDINLQNIPRRLRQFIGFETEDKKLITADFPQIELRLAGVIWNEPEFINAFRKGLDLHKLTASILFEKNIEEVSKEERQIGKSANFGLIYGISPRGFAEYCISNGINMTEEMAVEIVRKWKKFYRKIAEQQKKAYERFKYDEYVDNETWLNRPYRAYKPQDLLNYQIQGSGAELFKKAIILIKEVRPDLKLVNLVHDEIVAEALTDEAEDIAMLIKQKMEEAWDYCLEKAKEFGNKVSEIKLDIEKPNISHVWEKE

SEQ ID NO: 8 - protein, 877 aa, Artificial Sequence
  synthetic construct; PS-6739-Taq-mut
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESGGGGSGGGGSNIQKSILKPQPKALVEPVLCNSIDEIPAKFNEPIYFDLATDEDRPVLASIYQPHFERKVYCLNLLKEKPTRFKEWLLKFSEIRGWGLDFDLRVLGYTYEQLKDKKIVDVQLAIKVQHYERFRQNGTKGEGFRLDDVARDLFGIEYPMDKSKIRTTFKQNMYNTFSEQQLLYASLDAYIPHLLYEQLSSSTLNSLVYQLDQTAQKIVVETSQHGMPVKLKALEEEIYRLTQLRNQMQKEIPFNYNSPKQTAKFFGLDSSSKDVLMDLALQGNEMAKKVLEARQIEKSLTFAKDLYDLAKKSGGRIYGNFFTTTAPSGRMSCSDINLQNIPRRLRQFIGFDTEDKKLITADFPQIELRLAGVIWNEPKFIEAFRQGIDLHKLTASILFDKQSIDEVSKEERQIGKSANFGLIYGISPRGFAEHCITNGINITEEQAYEIVKKWKKYYTKITEQQKKAYERFKYNEYVDNETWLNRTYRAYKPQDLLNYQIQGSGAELFKKAIILLKQEEPSLKIVNLVHDEIVVEADSKDAQDLAKLIKEKMEEAWDWCLEKAEEFGNRVAKIKLEVEEPHVGEVWEKG

SEQ ID NO: 9 - DNA, 2631 bp, Artificial Sequence
  synthetic construct; Codon optimized O15 variant DNA sequence
atgcgtggtatgcttccactgtttgaaccgaaaggccgtgtgctgctggttgatggccaccatctggcctatcgtaccttccatgcgctgaaaggcctgacgaccagccgcggcgaaccggtgcaggcggtgtatggctttgcgaaaagcctgctgaaagcgctgaaagaagatggcgatgcggttattgtggtgtttgatgcgaaagcgccgagctttcgtcatgaagcgtatggcggctataaagcgggtcgtgcgccgaccccggaagattttccgcgtcagctggccctgattaaagaactggtggatctgctgggcctggcgcgtctggaagtgccgggctatgaagcggatgatgtgctggccagcctggccaaaaaagcggaaaaagaaggctacgaagttcgtattctgaccgccgataaagacctgtatcagctgctgtctgatcgtattcatgtgctgcatcctgagggttatctgattaccccggcgtggctgtgggaaaaatatggcctgcgtccggatcagtgggcggattatcgtgcgctgaccggcgatgaaagcgataacctgccgggcgtgaaaggcattggcgaaaaaaccgcgcgtaaactgctggaagaatggggcagcctggaagcgctgctgaaaaacctggatcgtctgaaaccggcgattcgtgaaaagatcttagcgcacatggatgatctgaaactgagctgggatctggccaaagtgcgtaccgatctgccgctggaagtggattttgcgaaacgtcgtgaaccggatcgtgaacgtctgcgtgcgtttctggaacgtctggaatttggcagcctgctgcatgaatttggcctgctggaaagcggtggcggcggttctggcggtggtggcagcaatactactacattaagtgtgaagcaggaggtaaaatcccttgttaaaccggtagtgtgcgattcgattgataaaattccagcaaagttcgatgaacccgtttattttgatcttgctaccgacaatgacaagcctgttttggcctctatctatcaatctcattttggacatgacgtctactgcttgaacttattaaaggagaaaccagcccgcctgaaagattggttgttgaaattcagcgagattcgtggctggggtttagattatgacttgcgcgttcttggctatacttatgaacaacttaaagacaaaaaaattgtagacgtacaacttgctattaaggtgcaacactacgaacgttttcgccagaacggagcgaagggcgagggtttcaagcttgacgatgtcgcccgcgacctgttgggaatcgaataccccatggacaagacgaaaatccgtactaccttcaagcaaaatatgtataattcttttaataaagaccagttattgtatgccagcctggatgcttacatccctcacttgctttacgagcaactgagttcaaatactttgaacagtttggtctatcagctggaccagcaagttcaaaagatcggcatcgagacgtcacaacatggtcttcctgtccgtctgcaagcattgcaagaagagattgataagttatcacagatcaagaaacgcattcagaaagagatcccattcaattataactcccctaaacaaaccacccagtacttgggcatcgatagctccagtaaggacgtgttgatggacctggcgttaaagggcaacgagttagctaagaaaatccttgaggctcgtcaaattgaaaaggctctgaccttcgctaaagagttatacgatttggcgaagcgtaataacggacgtatttacggtaacttctttactactaccgcgccatctgggcgtatgtcgtgtagcgacatcaacttgcaaaacattccacgcaagttgcgtccgttcattggctttgaaactgaagataagcgtctgattaccgctgattttccccaaatcgaattgcgcttggctggtgtaatctggaacgaaagtaagtttattgaagccttcaatcaaggaattgacttacacaagttgacagcatcaattctgttcggcaagcgctcggtcgatgaggtcagtaaagaagagcgccagatcgggaagtctgcaaactttgggttgatctatgggatctccccgcgtggattcgctgagtactgcatcactaatggaatcaacatgaccgaagagatcgcatacgagatcgtcaagaagtggaaacgttattatacaaaaatcactgaacaacaaaagaaggcgtatgaacgcttcaaatacggggagtacgtcgataacgaaacctggttagcccgtacctatcgtgcctataaaccccaggacttgttgaactaccagatccagggttctggggctgagctgttcaaaaaagctatcatcctgttgaaagaggaggagccaagtgttaaaattgtcaacttggtccatgatgaaatcgttgttgaggctgatagtaaagatgctcaggacgtagccaatttaattaaagaaaagatggggcaggcctgggattactgcttggataaggccaaagaattcggaaaccgcgtagcggaaattaagcttgaagtagaagagcccaatgtcagtgaagtttgggaaaagggc

SEQ ID NO: 10 - protein, 877 aa, Artificial Sequence
  synthetic construct; Engineered O15 variant polymerase
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESGGGGSGGGGSNTTTLSVKQEVKSLVKPVVCDSIDKIPAKFDEPVYFDLATDNDKPVLASIYQSHFGHDVYCLNLLKEKPARLKDWLLKFSEIRGWGLDYDLRVLGYTYEQLKDKKIVDVQLAIKVQHYERFRQNGAKGEGFKLDDVARDLLGIEYPMDKTKIRTTFKQNMYNSFNKDQLLYASLDAYIPHLLYEQLSSNTLNSLVYQLDQQVQKIGIETSQHGLPVRLQALQEEIDKLSQIKKRIQKEIPFNYNSPKQTTQYLGIDSSSKDVLMDLALKGNELAKKILEARQIEKALTFAKELYDLAKRNNGRIYGNFFTTTAPSGRMSCSDINLQNIPRKLRPFIGFETEDKRLITADFPQIELRLAGVIWNESKFIEAFNQGIDLHKLTASILFGKRSVDEVSKEERQIGKSANFGLIYGISPRGFAEYCITNGINMTEEIAYEIVKKWKRYYTKITEQQKKAYERFKYGEYVDNETWLARTYRAYKPQDLLNYQIQGSGAELFKKAIILLKEEEPSVKIVNLVHDEIVVEADSKDAQDVANLIKEKMGQAWDYCLDKAKEFGNRVAEIKLEVEEPNVSEVWEKG

SEQ ID NO: 11 - DNA, 2631 bp, Artificial Sequence
  synthetic construct; Codon optimized O57 variant DNA sequence
atgcgtggtatgcttccactgtttgaaccgaaaggccgtgtgctgctggttgatggccaccatctggcctatcgtaccttccatgcgctgaaaggcctgacgaccagccgcggcgaaccggtgcaggcggtgtatggctttgcgaaaagcctgctgaaagcgctgaaagaagatggcgatgcggttattgtggtgtttgatgcgaaagcgccgagctttcgtcatgaagcgtatggcggctataaagcgggtcgtgcgccgaccccggaagattttccgcgtcagctggccctgattaaagaactggtggatctgctgggcctggcgcgtctggaagtgccgggctatgaagcggatgatgtgctggccagcctggccaaaaaagcggaaaaagaaggctacgaagttcgtattctgaccgccgataaagacctgtatcagctgctgtctgatcgtattcatgtgctgcatcctgagggttatctgattaccccggcgtggctgtgggaaaaatatggcctgcgtccggatcagtgggcggattatcgtgcgctgaccggcgatgaaagcgataacctgccgggcgtgaaaggcattggcgaaaaaaccgcgcgtaaactgctggaagaatggggcagcctggaagcgctgctgaaaaacctggatcgtctgaaaccggcgattcgtgaaaagatcttagcgcacatggatgatctgaaactgagctgggatctggccaaagtgcgtaccgatctgccgctggaagtggattttgcgaaacgtcgtgaaccggatcgtgaacgtctgcgtgcgtttctggaacgtctggaatttggcagcctgctgcatgaatttggcctgctggaaagcggtggcggcggttctggcggtggtggcagcaatactactacattaagtgtgaagcaggaggtaaaatcccttgttaaaccggtagtgtgcgattcgattgataaaattccagcaaagttcgatgaacccgtttattttgatcttgctaccgacaatgacaagcctgttttggcctctatctatcaatctcattttggacatgacgtctactgcttgaacttattaaaggagaaaccagcccgcctgaaagattggttgttgaaattcagcgagattcgtggctggggtttagattatgacttgcgcgttcttggctatacttatgaacaacttaaagacaaaaaaattgtagacgtacaacttgctattaaggtgcaacactacgaacgttttcgccagaacggagcgaagggcgagggtttcaagcttgacgatgtcgcccgcgacctgttgggaatcgaataccccatggacaagacgaaaatccgtactaccttcaagcaaaatatgtataattcttttaataaagaccagttattgtatgccagcctggatgcttacatccctcacttgctttacgagcaactgagttcaaatactttgaacagtttggtctatcagctggaccagcaagttcaaaagatcggcatcgagacgtcacaacatggtcttcctgtccgtctgcaagcattgcaagaagagattgataagttatcacagatcaagaaacgcattcagaaagagatcccattcaattataactcccctaaacaaaccacccagtacttgggcatcgatagctccagtaaggacgtgttgatggacctggcgttaaagggcaacgagttagctaagaaaatccttgaggctcgtcaaattgaaaaggctctgaccttcgctaaagtgttatacgatttggcgaagcgtaataacggacgtatttacggtaacttctttactactaccgcgccatctgggcgtatgtcgtgtagcgacatcaacttgcaaaacattccacgcaagttgcgtccgttcattggctttgaaactgaagataagcgtctgattaccgctgattttccccaaatcgaattgcgcttggctggtgtaatctggaacgaaaagaagtttattgaagccttcaatcaaggaattgacttacacaagttgacagcatcaattctgttcggcaagcgctcggtcgatgaggtcagtaaagaagagcgccagatcgggaagtctgcaaactttgggttgatctatgggatctccccgcgtggattcgctgagtactgcatcactaatggaatcaacatgaccgaagagatcgcatacgagatcgtcaagaagtggaaagcgtattatacaaaaatcactgaacaacaaaagaaggcgtatgaacgcttcaaatacggggagtacgtcgataacgaaacctggttagcccgtacctatcgtgcctataaaccccaggacttgttgaactaccagatccagggttctggggctgagctgttcaaaaaagctatcatcctgttgaaagaggaggagccaagtgttaaaattgtcaacttggtccatgatgaaatcgttgttgaggctgatagtaaagatgctcaggacgtagccaatttaattaaagaaaagatggggcaggcctgggattactgcttggataaggccaaagaattcggaaaccgcgtagcggaaattaagcttgaagtagaagagcccaatgtcagtgaagtttgggaaaagggc

SEQ ID NO: 12 - protein, 877 aa, Artificial Sequence
  synthetic construct; Engineered O57 variant polymerase
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESGGGGSGGGGSNTTTLSVKQEVKSLVKPVVCDSIDKIPAKFDEPVYFDLATDNDKPVLASIYQSHFGHDVYCLNLLKEKPARLKDWLLKFSEIRGWGLDYDLRVLGYTYEQLKDKKIVDVQLAIKVQHYERFRQNGAKGEGFKLDDVARDLLGIEYPMDKTKIRTTFKQNMYNSFNKDQLLYASLDAYIPHLLYEQLSSNTLNSLVYQLDQQVQKIGIETSQHGLPVRLQALQEEIDKLSQIKKRIQKEIPFNYNSPKQTTQYLGIDSSSKDVLMDLALKGNELAKKILEARQIEKALTFAKVLYDLAKRNNGRIYGNFFTTTAPSGRMSCSDINLQNIPRKLRPFIGFETEDKRLITADFPQIELRLAGVIWNEKKFIEAFNQGIDLHKLTASILFGKRSVDEVSKEERQIGKSANFGLIYGISPRGFAEYCITNGINMTEEIAYEIVKKWKAYYTKITEQQKKAYERFKYGEYVDNETWLARTYRAYKPQDLLNYQIQGSGAELFKKAIILLKEEEPSVKIVNLVHDEIVVEADSKDAQDVANLIKEKMGQAWDYCLDKAKEFGNRVAEIKLEVEEPNVSEVWEKG

SEQ ID NO: 13 - DNA, 2631 bp, Artificial Sequence
  synthetic construct; Codon optimized O58 variant DNA sequence
atgcgtggtatgcttccactgtttgaaccgaaaggccgtgtgctgctggttgatggccaccatctggcctatcgtaccttccatgcgctgaaaggcctgacgaccagccgcggcgaaccggtgcaggcggtgtatggctttgcgaaaagcctgctgaaagcgctgaaagaagatggcgatgcggttattgtggtgtttgatgcgaaagcgccgagctttcgtcatgaagcgtatggcggctataaagcgggtcgtgcgccgaccccggaagattttccgcgtcagctggccctgattaaagaactggtggatctgctgggcctggcgcgtctggaagtgccgggctatgaagcggatgatgtgctggccagcctggccaaaaaagcggaaaaagaaggctacgaagttcgtattctgaccgccgataaagacctgtatcagctgctgtctgatcgtattcatgtgctgcatcctgagggttatctgattaccccggcgtggctgtgggaaaaatatggcctgcgtccggatcagtgggcggattatcgtgcgctgaccggcgatgaaagcgataacctgccgggcgtgaaaggcattggcgaaaaaaccgcgcgtaaactgctggaagaatggggcagcctggaagcgctgctgaaaaacctggatcgtctgaaaccggcgattcgtgaaaagatcttagcgcacatggatgatctgaaactgagctgggatctggccaaagtgcgtaccgatctgccgctggaagtggattttgcgaaacgtcgtgaaccggatcgtgaacgtctgcgtgcgtttctggaacgtctggaatttggcagcctgctgcatgaatttggcctgctggaaagcggtggcggcggttctggcggtggtggcagcaatactactacattaagtgtgaagcaggaggtaaaatcccttgttaaaccggtagtgtgcgattcgattgataaaattccagcaaagttcgatgaacccgtttattttgatcttgctaccgacaatgacaagcctgttttggcctctatctatcaatctcattttggacatgacgtctactgcttgaacttattaaaggagaaaccagcccgcctgaaagattggttgttgaaattcagcgagattcgtggctggggtttagattatgacttgcgcgttcttggctatacttatgaacaacttaaagacaaaaaaattgtagacgtacaacttgctattaaggtgcaacactacgaacgttttcgccagaacggagcgaagggcgagggtttcaagcttgacgatgtcgcccgcgacctgttgggaatcgaataccccatggacaagacgaaaatccgtactaccttcaagcaaaatatgtataattcttttaataaagaccagttattgtatgccagcctggatgcttacatccctcacttgctttacgagcaactgagttcaaatactttgaacagtttggtctatcagctggaccagcaagttcaaaagatcggcatcgagacgtcacaacatggtcttcctgtccgtctgcaagcattgcaagaagagattgataagttatcacagatcaagaaacgcattcagaaagagatcccattcaattataactcccctaaacaaaccacccagtacttgggcatcgatagctccagtaaggacgtgttgatggacctggcgttaaagggcaacgagttagctaagaaaatccttgaggctcgtcaaattgaaaaggctctgaccttcgctaaagagttatacgatttggcgaagcgtaataacggacgtatttacggtaacttctttactactaccgcgccatctgggcgtatgtcgtgtagcgacatcaacttgcaaaacattccacgcaagttgcgtccgttcattggctttgaaactgaagataagcgtctgattaccgctgattttccccaaatcgaattgcgcttggctggtgtaatctggaacgaaaagaagtttattgaagccttcaatcaaggaattgacttacacaagttgacagcatcaattctgttcgaaaagcgctcggtcgatgaggtcagtaaagaagagcgccagatcgggaagtctgcaaactttgggttgatctatgggatctccccgcgtggattcgctgagtactgcatcactaatggaatcaacatgaccgaagagatcgcatacgagatcgtcaagaagtggaaacgttattatacaaaaatcactgaacaacaaaagaaggcgtatgaacgcttcaaatacggggagtacgtcgataacgaaacctggttagcccgtacctatcgtgcctataaaccccaggacttgttgaactaccagatccagggttctggggctgagctgttcaaaaaagctatcatcctgttgaaagaggaggagccaagtgttaaaattgtcaacttggtccatgatgaaatcgttgttgaggctgatagtaaagatgctcaggacgtagccaatttaattaaagaaaagatggggcaggcctgggattactgcttggataaggccaaagaattcggaaaccgcgtagcggaaattaagcttgaagtagaagagcccaatgtcagtgaagtttgggaaaagggc

SEQ ID NO: 14 - protein, 877 aa, Artificial Sequence
  synthetic construct; Engineered O58 variant polymerase
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESGGGGSGGGGSNTTTLSVKQEVKSLVKPVVCDSIDKIPAKFDEPVYFDLATDNDKPVLASIYQSHFGHDVYCLNLLKEKPARLKDWLLKFSEIRGWGLDYDLRVLGYTYEQLKDKKIVDVQLAIKVQHYERFRQNGAKGEGFKLDDVARDLLGIEYPMDKTKIRTTFKQNMYNSFNKDQLLYASLDAYIPHLLYEQLSSNTLNSLVYQLDQQVQKIGIETSQHGLPVRLQALQEEIDKLSQIKKRIQKEIPFNYNSPKQTTQYLGIDSSSKDVLMDLALKGNELAKKILEARQIEKALTFAKELYDLAKRNNGRIYGNFFTTTAPSGRMSCSDINLQNIPRKLRPFIGFETEDKRLITADFPQIELRLAGVIWNEKKFIEAFNQGIDLHKLTASILFEKRSVDEVSKEERQIGKSANFGLIYGISPRGFAEYCITNGINMTEEIAYEIVKKWKRYYTKITEQQKKAYERFKYGEYVDNETWLARTYRAYKPQDLLNYQIQGSGAELFKKAIILLKEEEPSVKIVNLVHDEIVVEADSKDAQDVANLIKEKMGQAWDYCLDKAKEFGNRVAEIKLEVEEPNVSEVWEKG

SEQ ID NO: 15 - protein, 876 aa, Artificial Sequence
  synthetic construct; OS-1622-Taq-wt - OS-1622 Taq nuclease domain fusion (without mutation)
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESGGGGSGGGGSNIPKPILKPQPKALVEPVLCDSVDEIPTKFNEPIYFDLATDGDRPVLASIYQPHFERKVYCLNLLKEKPTRFKEWLLKFSEIRGWGLDFDLRALGYTYEQLRDKKIVDVQLAIKVQHHERFKQNGTKGEGFRLDDVARDLLGIEYPMDKTKIRETFKNNIFHSFSNEQLLYASLDAYIPHLLYEQLTSSTLNSLVYQLDQQAQKIVVETSQNGMPVKLKALEEEIHRLTQLRNQMQKEIPFNYNSPKQTAKFFRVDSSSKDVLMDLALQGNEMAKRVLEARQVEKSLAFAKDLYDIAKRSGGRVYGNFFTTTAPSGRMSCSDINLQQIPRRLRQFIGFDTEDKRLITADFPQIELRLAGVIWNESEFIEAFKQGIDLHKLTASILFEKNIEEVGKEERQIGKSANFGLIYGIAPKGFAEYCITNGINMTEEQAYEIVRKWKKYYTKIAEQHQVAYERFKYNEYVDNETWLNRTYRAWKPQDLLNYQIQGSGAELFKKAIVLLKEAKPDLKIVNLVHDEIVVEADSKEAQDLAKLIKEKMEEAWDWCLEKAEEFGNRVAKIKLEVEQPNVGDTWEKS

SEQ ID NO: 16 - protein, 877 aa, Artificial Sequence
  synthetic construct; OP-2605-Taq-wt - OP-2605 Taq nuclease domain fusion (without mutation)
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESGGGGSGGGGSNTTTLSVKQEVKSLVKPVVCDSIDKIPAKFDEPVYFDLATDNDKPVLASIYQSHFGHDVYCLNLLKEKPARLKDWLLKFSEIRGWGLDYDLRVLGYTYEQLKDKKIVDVQLAIKVQHYERFRQNGAKGEGFKLDDVARDLLGIEYPMDKTKIRTTFKQNMYNSFNKDQLLYASLDAYIPHLLYEQLSSNTLNSLVYQLDQQVQKIGIETSQHGLPVRLQALQEEIDKLSQIKKRIQKEIPFNYNSPKQTTQYLGIDSSSKDVLMDLALKGNELAKKILEARQIEKALTFAKDLYDLAKRNNGRIYGNFFTTTAPSGRMSCSDINLQQIPRKLRPFIGFETEDKKLITADFPQIELRLAGVIWNEPKFIEAFNQGIDLHKLTASILFDKRSVDEVSKEERQIGKSANFGLIYGISPKGFAEYCITNGINMTEEIAYEIVKKWKKYYTKITEQHQVAYERFKYGEYVDNETWLNRTYRAYKPQDLLNYQIQGSGAELFKKAIILLKEEEPSVKIVNLVHDEIVVEADSKDAQDVANLIKEKMGQAWDYCLDKAKEFGNRVAEIKLEVEEPNVSEVWEKG

SEQ ID NO: 17 - protein, 877 aa, Artificial Sequence
  synthetic construct; CS-2729-Taq-wt - CS-2729 Taq nuclease domain fusion (without mutation)
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESGGGGSGGGGSNTPFTVKVKPANKSLVDPILCNSIDEIPVRYDEPVYFDIATEEDKPVLVSVYQPHFGNKVYCLNLLREKPARFKEWFLKFSEIRGWGLDFDLKILGYTYEQLKNKKIVDVQLAIKVQHYERFKQGGTKGEGFRLDEVARDLLGIEYPMDKSKIRMTFRNNMFSSFSYEQLLYASLDAYIPHLLYERLSSSTLNSLVYQIDQEVQKIVVETSQHGMPVKLQALEEEIHRLLQIKNQIQKEIPFNYNSPQQTAKFFGVNSSSKDVLMDLVLKGNEMAKKVLEARQVEKSLAFAKDLYDLAKRSGGRIYGNFFTTTAPSGRMSCSDINLQQIPRRLRQFIGFETEDKKLITADFPQIELRLAGVIWNEPEFINAFRKGLDLHKLTASILFEKNIEEVSKEERQIGKSANFGLIYGISPRGFAEYCISNGINMTEEMAVEIVRKWKKFYRKIAEQHQLAYERFKYDEYVDNETWLNRPYRAYKPQDLLNYQIQGSGAELFKKAIILIKEVRPDLKLVNLVHDEIVAEALTDEAEDIAMLIKQKMEEAWDYCLEKAKEFGNKVSEIKLDIEKPNISHVWEKE

SEQ ID NO: 18 - protein, 877 aa, Artificial Sequence
  synthetic construct; PS-6739-Taq-wt - PS-6739 Taq nuclease domain fusion (without mutation)
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESGGGGSGGGGSNIQKSILKPQPKALVEPVLCNSIDEIPAKFNEPIYFDLATDEDRPVLASIYQPHFERKVYCLNLLKEKPTRFKEWLLKFSEIRGWGLDFDLRVLGYTYEQLKDKKIVDVQLAIKVQHYERFRQNGTKGEGFRLDDVARDLFGIEYPMDKSKIRTTFKQNMYNTFSEQQLLYASLDAYIPHLLYEQLSSSTLNSLVYQLDQTAQKIVVETSQHGMPVKLKALEEEIYRLTQLRNQMQKEIPFNYNSPKQTAKFFGLDSSSKDVLMDLALQGNEMAKKVLEARQIEKSLTFAKDLYDLAKKSGGRIYGNFFTTTAPSGRMSCSDINLQQIPRRLRQFIGFDTEDKKLITADFPQIELRLAGVIWNEPKFIEAFRQGIDLHKLTASILFDKQSIDEVSKEERQIGKSANFGLIYGISPRGFAEHCITNGINITEEQAYEIVKKWKKYYTKITEQHQIAYERFKYNEYVDNETWLNRTYRAYKPQDLLNYQIQGSGAELFKKAIILLKQEEPSLKIVNLVHDEIVVEADSKDAQDLAKLIKEKMEEAWDWCLEKAEEFGNRVAKIKLEVEEPHVGEVWEKG

SEQ ID NO: 19 - DNA, 10 bp, Artificial Sequence
  synthetic construct; linker
ggggsggggs

SEQ ID NO: 20 - protein, 1607 aa, Artificial Sequence
  synthetic construct; Putative viral gene product. Locus tag JGI20132J14458_100001622
MRSISFFELLVKIGLIVEDEYGYTFPDYVLVLTQTPEGIELKEIKDAFLRWNETNKEKWVEEFEEYCKLARERNRYYLSLFAEKRNAQDFFKRTKVAIRIDIDEPLKLDQILEIVNNRELLPIQPTHILRTIKGWHIFYITQDFIECDDKEILYMIHSYVEDLKSNLRKHADKIDHTYSIATRYSNEIYELREPYTKKELLEEMNKYYDTDILINGLPVKRREYSRIPISQISEGLALTLWNACPVIRSLEEKWETHTYNEWFILSWKYAFLYVLTQKEEYKQEFLQKSKFWKGKVVIAPEQQFRNTLKWMLKDRETLPYFSCSFVHRRVVDADEKCKNCQYARWIFDEYGERKLISNWFKDLFYLETRLEGFKVDEKRNLWVKEDTNEPVCELFKIEDVVLYNKPNRKEKYIKIFYRDKYEFIPYVLTASANTDFSEFIVLTFYNQQLFKKLLTNYLTLFQLARGVREIDKAGYKYNDLKRKWDMVVANMDSFRAEDLNFYMWSDRTNRLNYYIPIVNGSFEAWKNAYRRVVKAKDPIMLILLGHFISHITKEYFRDKFVASSEPNVLIFLRGFTTTGKTTRLRIASALYGTPQVIQITETTTAKILREFGNIGMPLPLDEFRMRKDKEEEIANMIYAIANEASKDTAYERFSPIQVPVVFSGEKNALAVEVLCKNREGLYRRSIVLDVDELPKQKNTALVEFYTNEILPILKYNHGYIFKLIDFIENHVDIEALAQYYKDVEILRNEFDKKRSKVLRGIVKSLDNHLKLIYASIHVFLEFLGLSDEEKANVFVILEQYIRNVFAKFYDTLLPKEESKLNKIIDYLRDLADGLYNASNNPIKKTTIRGLTIKKLIDIAGVQVPTTDIEPYLKLLFMKYYENKKAFVYLGSIFVEGRNPAWFEGMVTREYERLTYIKEHHPEFYKSILEVFTELMLSIHGEAGLRRLHNLFVESFKFEDLKDFIDNNGGDNTPPDEDLPSGDDDDNTPPNDNLPPVEEFDYENKENEDNEEEDELEKHFTGEDGLSLPKRMNIPKPILKPQPKALVEPVLCDSVDEIPTKFNEPIYFDLETDGDRPVLASIYQPHFERKVYCLNLLKEKPTRFKEWLLKFSEIRGWGLDFDLRALGYTYEQLRDKKIVDVQLAIKVQHHERFKQNGTKGEGFRLDDVARDLLGIEYPMDKTKIRETFKNNIFHSFSNEQLLYASLDAYIPHLLYEQLTSSTLNSLVYQLDQQAQKIVVETSQNGMPVKLKALEEEIHRLTQLRNQMQKEIPFNYNSPKQTAKFFRVDSSSKDVLMDLALQGNEMAKRVLEARQVEKSLAFAKDLYDIAKRSGGRVYGNFFTTTAPSGRMSCSDINLQQIPRRLRQFIGFDTEDKRLITADFPQIELRLAGVIWNESEFIEAFKQGIDLHKLTASILFEKNIEEVGKEERQIGKSANFGLIYGIAPKGFAEYCITNGINMTEEQAYEIVRKWKKYYTKIAEQHQVAYERFKYNEYVDNETWLNRTYRAWKPQDLLNYQIQGSGAELFKKAIVLLKEAKPDLKIVNLVHDEIVVEADSKEAQDLAKLIKEKMEEAWDWCLEKAEEFGNRVAKIKLEVEQPNVGDTWEKS

SEQ ID NO: 21 - protein, 1595 aa, Artificial Sequence
  synthetic construct; Putative viral gene product. Locus tag Ga0186926_122605
MNKITFFDLFVKIGLVYENEKYGYTFNDYVLVLAETLEGVAVKEIRDAFLGFNEADKERWKKEFEEYCKVARERNRYFLSLFAEKRNSFDYFKRTKVAIRIDIDEPLKLEEVLELVNNRDLIPIPPTHILRSVKGWHIFYITQDYIESVDREVLYFIHSYTEELKSLLRKHADKVDHTYQIATRFSEEIYELREPYTKEKLFQAINDYYGVEIQINGLTVKRGQYGKIPVAHLSEGVALTLWNACPVLRQLEERWENHTYDEWFLMSWKYAFLYALTQKEEYKQEFLQKSKLWKGQVKTTPEQQFQYTLKWILKDRETLPYFSCSFVHKSVEGAEEKCNSCQYARWMLDENGERRLISNWFKDLFYLETRLEGFKIDERKNVWVKEDTEEPVCELFKIEDVVLYNKPNNKQKYIKIFYRDKYEFIPYVLTASANTDFSEFIVLTFYNQQLFKKLLTNYLTLFQLARGVREIDKAGYKYNDLKKRWDTVVANVGAFRVEDLNFYMWNDRTSRLNYYIPVVNGSFEAWKDAYRRVVKAKDPILLILLGHFISHITKEYFKDKFVASSEPNVLIFLRGFTTAGKTTRLRIASALYGTPQAIQITETTTAKILREFGNIGTPLPLDEFRMRKDKEEEVANMIYAIANESAKDTAYERFNPIQVPVVFSGEKNALSVETLCKNRDGLYRRSIVLDIDEIPKQKNSSLVEFYTNKILPILKYHHGYIFKFIDFIENEVDIETVAERFKDVELLNEELNKKKSKVFRGIVKSLDNHLKMIIASLSVFLDFLNLNEEEKADIYIALDHYIRNVLAKFYDTLLPKEEDKLSKIIDYLRDFADGLYNASNNPIKKTTIKGLTTKKLIDVAGMQVPTTDIEPYLRLLFMKYYQSNRGYTYLGSIFVEGRNPAWFESMIKIEYERLIHIKEQHPTYYKNALEVFVELMLSIHGELGLRRLYRIFVKTYKFDDLKDFISDNNDDTPPDDNPPNGDDGDDDLPPDDSISPNGHYTEDPEEPHFEEETNSFSQNTTTLSVKQEVKSLVKPVVCDSIDKIPAKFDEPVYFDLETDNDKPVLASIYQSHFGHDVYCLNLLKEKPARLKDWLLKFSEIRGWGLDYDLRVLGYTYEQLKDKKIVDVQLAIKVQHYERFRQNGAKGEGFKLDDVARDLLGIEYPMDKTKIRTTFKQNMYNSFNKDQLLYASLDAYIPHLLYEQLSSNTLNSLVYQLDQQVQKIGIETSQHGLPVRLQALQEEIDKLSQIKKRIQKEIPFNYNSPKQTTQYLGIDSSSKDVLMDLALKGNELAKKILEARQIEKALTFAKDLYDLAKRNNGRIYGNFFTTTAPSGRMSCSDINLQQIPRKLRPFIGFETEDKKLITADFPQIELRLAGVIWNEPKFIEAFNQGIDLHKLTASILFDKRSVDEVSKEERQIGKSANFGLIYGISPKGFAEYCITNGINMTEEIAYEIVKKWKKYYTKITEQHQVAYERFKYGEYVDNETWLNRTYRAYKPQDLLNYQIQGSGAELFKKAIILLKEEEPSVKIVNLVHDEIVVEADSKDAQDVANLIKEKMGQAWDYCLDKAKEFGNRVAEIKLEVEEPNVSEVWEKG

SEQ ID NO: 22 - protein, 1619 aa, Artificial Sequence
  synthetic construct; Putative viral gene product. Locus tag Ga0080008_15802729
MNRITFFDLFVKCGLIYDDEEYGYRFTPYVLVLAETVDGIGIKPITDLFFGFNETDRERWVKEFLSYCKEARERNRYYLSVFSERRNSFDFFKRTKAAIRIDIDEPLTLSEVIKLVENKDLIPIQPTHVLRSVRGWHILYITKDFIENDEQNKNIFYLLHSYAEDLKSNLRKYADKVDYTYQIATRFSEEIYELREPYEVKELIKAIEDYYSLDIEINGFKLKRRQFGRIPISHISEGVALTLWNACPVLRRLEEKWEYHTYNEWFIMSWKYAFLYALTGKSEYKEEFLNKSKLWKGVVKMTPEQQFEYTLKWVLKEKETLPYFSCSFVYKHVSEAEEKCKECPYARWQEDEFGNKTLISSWFKELFYIESRLENFKIDEKRNLWVKADTNEPICELFKIEDVVLYNKPNKKERFIKIFYRNKYEFVPYVLTASANMDFSEFNVLTFYNQTLFKNLLINYLNLFQLSRGAREIDKAGYKYNRITKSWDKVVANLGNFRVEDLNFFMWNDRTNELRYYIPVVNGSYEVWRETYKKVLLAKDPIMLIILGHFLSHITREYFKDKFVSSNEPNVLIFLRGFTTSGKTTRLKIASALYGTPEVIQITETTTAKILREFGNIGMPLPLDEFRMRKDKEEEVANMIYAIANEAAKDTAYERFNPISVPVVFSGEKNTLFVETLAKNREGLYRRSIVLDVDEIPKPEREQLAEFYAREIYPVLRKNHGFIYKFIEFLENEADIDRLSELYQDVELLREEFDKRRSKVLRGIVRSLDNHLKMILASLHLFVDFIGLNDEEKAEVYMCVEDYIKTKLVGFYETFLPKEEDKLTRIIDYLRDIIDGLYNAWKHPVNKKTIKRLTINKLIEIAGVQAPTQDLEPYLKLLLMKYYPSNNTFTYVGSVFVEGRNYLSDDYAKLETERLLFVKGRYPHLYQDILEVFVELMLIVHGEYGLSKLIKYMKKLGFTDVMEYTIKHNITIHKFGDDEDDNPSPTSPPKNPPEISPQNNSSSTEITSTSEVDEDLVNSFVGEEGFSSATLKTDTTKQQNQTNTPFTVKVKPANKSLVDPILCNSIDEIPVRYDEPVYFDIETEEDKPVLVSVYQPHFGNKVYCLNLLREKPARFKEWFLKFSEIRGWGLDFDLKILGYTYEQLKNKKIVDVQLAIKVQHYERFKQGGTKGEGFRLDEVARDLLGIEYPMDKSKIRMTFRNNMFSSFSYEQLLYASLDAYIPHLLYERLSSSTLNSLVYQIDQEVQKIVVETSQHGMPVKLQALEEEIHRLLQIKNQIQKEIPFNYNSPQQTAKFFGVNSSSKDVLMDLVLKGNEMAKKVLEARQVEKSLAFAKDLYDLAKRSGGRIYGNFFTTTAPSGRMSCSDINLQQIPRRLRQFIGFETEDKKLITADFPQIELRLAGVIWNEPEFINAFRKGLDLHKLTASILFEKNIEEVSKEERQIGKSANFGLIYGISPRGFAEYCISNGINMTEEMAVEIVRKWKKFYRKIAEQHQLAYERFKYDEYVDNETWLNRPYRAYKPQDLLNYQIQGSGAELFKKAIILIKEVRPDLKLVNLVHDEIVAEALTDEAEDIAMLIKQKMEEAWDYCLEKAKEFGNKVSEIKLDIEKPNISHVWEKE

SEQ ID NO: 23 - protein, 1608 aa, Artificial Sequence
  synthetic construct; Putative viral gene product. Locus tag Ga0079997_11796739
MKSISFSELFVKIGLVSETDDGYTFNDYVLVLSQTPEGTVLKEIREAFLGFNETDKERWVKEFEEYCKEARERNRYYLSLFAEKRNSQDYLKRTKVAIRIDIDEPLKLEQVLEIVNNGDLIPIPPTHLLRTIKGWHIFYITKDFIENEDKEVIYLIHSYTEELKTHLRKYADKIDHTYQIATRYSTEIYELREPYTKEELLKAINDYFGVEIQVNGLIVKRKDCSGVPVSQLSEGLALTLWNACPVLRSLEERWETHTYHEWFILSWKHAFLYVLTQKEEYRQEFLQKSKLWKGKVVITPEQQFQNTLKWMLKDRETLPYFSCSFVYKYVADAGEKCEKCQYARWVFDENGERKLISNWFRDLFYLETRLEGFRVDEKRNLWVKEDTGEPVCELFKIEDVVLYNKPNRKEKYIKIFYRDKYEFIPYVLTASANTDFSEFIVLTFYNQQLFKYLLNKYLTLFQLARGVREIDKAGYKYNDLKRKWDMVVANMGSFRAEDLNFYMWNDRTNRLNYYIPIMNGSFETWKNTYRRVVKAKDPIMLLLLGHFISHITKEYFRDKFVASSEPNVLIFLRGFTTAGKTTRLRIASALYGTPQVIQITETTTAKILREFGNIGMPLPLDEFKMRKDKEEEVANMIYAIANEASKDTAYERFNPIQVPVVFSGEKNALSVEKLCANREGLYRRSIVLDVDELPKQKNSALIDFYTSELLPILKYNHGYIFKLIDFIENNLDIEALTQLYKDVEILKDEFDKRKSKALRGIVKSLDNHLKLIFASIHVFLEFLDLSEEEKAEVFAILEEYIRNVLAKFYDTLLPKEENKLSKIVDYLRDLADGLYNASNNPIKKTTIRGLTLKKLIDVAGVQVPTTDIEPYVKMLFMRYYESKKGYVYLGSIFVEGRNPAWFEGMVAREYERLIYIKQHYPELYRSILEVFAELMLSIHGEAGLRRVHSIFVESFKFDDLKDFLNNNNDDNTPPDDLPPNGGDDDDTPPDDLPPTEEFDYENEEDEEDEEEEDELNEHFAGEDGLTTPKMMNIQKSILKPQPKALVEPVLCNSIDEIPAKFNEPIYFDLETDEDRPVLASIYQPHFERKVYCLNLLKEKPTRFKEWLLKFSEIRGWGLDFDLRVLGYTYEQLKDKKIVDVQLAIKVQHYERFRQNGTKGEGFRLDDVARDLFGIEYPMDKSKIRTTFKQNMYNTFSEQQLLYASLDAYIPHLLYEQLSSSTLNSLVYQLDQTAQKIVVETSQHGMPVKLKALEEEIYRLTQLRNQMQKEIPFNYNSPKQTAKFFGLDSSSKDVLMDLALQGNEMAKKVLEARQIEKSLTFAKDLYDLAKKSGGRIYGNFFTTTAPSGRMSCSDINLQQIPRRLRQFIGFDTEDKKLITADFPQIELRLAGVIWNEPKFIEAFRQGIDLHKLTASILFDKQSIDEVSKEERQIGKSANFGLIYGISPRGFAEHCITNGINITEEQAYEIVKKWKKYYTKITEQHQIAYERFKYNEYVDNETWLNRTYRAYKPQDLLNYQIQGSGAELFKKAIILLKQEEPSLKIVNLVHDEIVVEADSKDAQDLAKLIKEKMEEAWDWCLEKAEEFGNRVAKIKLEVEEPHVGEVWEKG

SEQ ID NO: 24 - protein, 576 aa, Artificial Sequence
  synthetic construct; Core family A polymerase OS-1622
NIPKPILKPQPKALVEPVLCDSVDEIPTKFNEPIYFDLETDGDRPVLASIYQPHFERKVYCLNLLKEKPTRFKEWLLKFSEIRGWGLDFDLRALGYTYEQLRDKKIVDVQLAIKVQHHERFKQNGTKGEGFRLDDVARDLLGIEYPMDKTKIRETFKNNIFHSFSNEQLLYASLDAYIPHLLYEQLTSSTLNSLVYQLDQQAQKIVVETSQNGMPVKLKALEEEIHRLTQLRNQMQKEIPFNYNSPKQTAKFFRVDSSSKDVLMDLALQGNEMAKRVLEARQVEKSLAFAKDLYDIAKRSGGRVYGNFFTTTAPSGRMSCSDINLQQIPRRLRQFIGFDTEDKRLITADFPQIELRLAGVIWNESEFIEAFKQGIDLHKLTASILFEKNIEEVGKEERQIGKSANFGLIYGIAPKGFAEYCITNGINMTEEQAYEIVRKWKKYYTKIAEQHQVAYERFKYNEYVDNETWLNRTYRAWKPQDLLNYQIQGSGAELFKKAIVLLKEAKPDLKIVNLVHDEIVVEADSKEAQDLAKLIKEKMEEAWDWCLEKAEEFGNRVAKIKLEVEQPNVGDTWEKS

SEQ ID NO: 25 - protein, 577 aa, Artificial Sequence
  synthetic construct; Core family A polymerase OP-2605
NTTTLSVKQEVKSLVKPVVCDSIDKIPAKFDEPVYFDLETDNDKPVLASIYQSHFGHDVYCLNLLKEKPARLKDWLLKFSEIRGWGLDYDLRVLGYTYEQLKDKKIVDVQLAIKVQHYERFRQNGAKGEGFKLDDVARDLLGIEYPMDKTKIRTTFKQNMYNSFNKDQLLYASLDAYIPHLLYEQLSSNTLNSLVYQLDQQVQKIGIETSQHGLPVRLQALQEEIDKLSQIKKRIQKEIPFNYNSPKQTTQYLGIDSSSKDVLMDLALKGNELAKKILEARQIEKALTFAKDLYDLAKRNNGRIYGNFFTTTAPSGRMSCSDINLQQIPRKLRPFIGFETEDKKLITADFPQIELRLAGVIWNEPKFIEAFNQGIDLHKLTASILFDKRSVDEVSKEERQIGKSANFGLIYGISPKGFAEYCITNGINMTEEIAYEIVKKWKKYYTKITEQHQVAYERFKYGEYVDNETWLNRTYRAYKPQDLLNYQIQGSGAELFKKAIILLKEEEPSVKIVNLVHDEIVVEADSKDAQDVANLIKEKMGQAWDYCLDKAKEFGNRVAEIKLEVEEPNVSEVWEKG

SEQ ID NO: 26 - protein, 577 aa, Artificial Sequence
  synthetic construct; ore family A polymerase CS-2729
NTPFTVKVKPANKSLVDPILCNSIDEIPVRYDEPVYFDIETEEDKPVLVSVYQPHFGNKVYCLNLLREKPARFKEWFLKFSEIRGWGLDFDLKILGYTYEQLKNKKIVDVQLAIKVQHYERFKQGGTKGEGFRLDEVARDLLGIEYPMDKSKIRMTFRNNMFSSFSYEQLLYASLDAYIPHLLYERLSSSTLNSLVYQIDQEVQKIVVETSQHGMPVKLQALEEEIHRLLQIKNQIQKEIPFNYNSPQQTAKFFGVNSSSKDVLMDLVLKGNEMAKKVLEARQVEKSLAFAKDLYDLAKRSGGRIYGNFFTTTAPSGRMSCSDINLQQIPRRLRQFIGFETEDKKLITADFPQIELRLAGVIWNEPEFINAFRKGLDLHKLTASILFEKNIEEVSKEERQIGKSANFGLIYGISPRGFAEYCISNGINMTEEMAVEIVRKWKKFYRKIAEQHQLAYERFKYDEYVDNETWLNRPYRAYKPQDLLNYQIQGSGAELFKKAIILIKEVRPDLKLVNLVHDEIVAEALTDEAEDIAMLIKQKMEEAWDYCLEKAKEFGNKVSEIKLDIEKPNISHVWEKE

SEQ ID NO: 27 - protein, 577 aa, Artificial Sequence
  synthetic construct; Core family A polymerase PS-6739
NIQKSILKPQPKALVEPVLCNSIDEIPAKFNEPIYFDLETDEDRPVLASIYQPHFERKVYCLNLLKEKPTRFKEWLLKFSEIRGWGLDFDLRVLGYTYEQLKDKKIVDVQLAIKVQHYERFRQNGTKGEGFRLDDVARDLFGIEYPMDKSKIRTTFKQNMYNTFSEQQLLYASLDAYIPHLLYEQLSSSTLNSLVYQLDQTAQKIVVETSQHGMPVKLKALEEEIYRLTQLRNQMQKEIPFNYNSPKQTAKFFGLDSSSKDVLMDLALQGNEMAKKVLEARQIEKSLTFAKDLYDLAKKSGGRIYGNFFTTTAPSGRMSCSDINLQQIPRRLRQFIGFDTEDKKLITADFPQIELRLAGVIWNEPKFIEAFRQGIDLHKLTASILFDKQSIDEVSKEERQIGKSANFGLIYGISPRGFAEHCITNGINITEEQAYEIVKKWKKYYTKITEQHQIAYERFKYNEYVDNETWLNRTYRAYKPQDLLNYQIQGSGAELFKKAIILLKQEEPSLKIVNLVHDEIVVEADSKDAQDLAKLIKEKMEEAWDWCLEKAEEFGNRVAKIKLEVEEPHVGEVWEKG